(19) United States
(12) Patent Application Publication
(10) Pub. No.: US 2021/0128466 A1
(43) Pub. Date: May 6, 2021

(54) REDUCED VOLUME FORMULATIONS INCLUDING AMINO ACID ENTITIES

(71) Applicant: AXCELLA HEALTH INC., Cambridge, MA (US)

(72) Inventors: Jason Legassie, Saugus, MA (US); Thomas Hanlon, Concord, MA (US); Ralph Yamamoto, Woburn, MA (US); Daniel Rothenberg, Cambridge, MA (US); Weijia Niu, Somerville, MA (US); Benjamin Farina, Somerville, MA (US)

(21) Appl. No.: 17/067,187

(22) Filed: Oct. 9, 2020

Related U.S. Application Data
(60) Provisional application No. 62/913,515, filed on Oct. 10, 2019.
(60) Provisional application No. 62/913,524, filed on Oct. 10, 2019.

(51) Int. Cl. A61K 9/10; A61K 47/24; A61K 47/34; A61K 31/19; A61K 31/205; A61K 31/198; A61K 47/02

(52) U.S. Cl. CPC A61K 9/10; A61K 47/24; A61K 47/34; A61K 47/02; A61K 31/205; A61K 31/198; A61K 31/19

(57) ABSTRACT

This disclosure provides compositions and methods for improving the dispersion of a hydrophobic amino acid entity in an aqueous suspension. This disclosure further provides compositions and methods for improving the stability of N-acetylcysteine in the presence of acetyl acceptors, e.g., carnitine.

### CROSS-REFERENCE TO RELATED APPLICATIONS

This application claims priority under 35 U.S.C. § 119(e) to U.S. Provisional Patent Application No. 62/913,515, filed Oct. 10, 2019 and U.S. Provisional Patent Application No. 62/913,524, filed Oct. 10, 2019, the disclosures of which are hereby incorporated herein by reference in their entireties for all purposes.

### BACKGROUND

Mixtures of amino acids have a number of uses relating to human health and disease treatment. Some amino acids have limited solubility and/or dispersion when added to aqueous mixtures at higher concentrations. Inefficient/limited dispersion or solubilization can lead to problems with aqueous suspensions of amino acids, including uneven distribution of amino acids in aqueous suspensions and partial dose delivery. For applications where large dose oral delivery of amino acids may be desirable, previous approaches have used high volumes to disperse the amino acids, or spray drying or spray coating to promote wetting of the amino acids. These approaches may add cost and complexity to the manufacture and use of large dose amino acid compositions. The above problems can be exacerbated by aqueous suspensions having low volumes. There is a need for techniques and compositions to improve the solubilization and/or dispersion of amino acids in aqueous suspensions.

### SUMMARY OF THE INVENTION

Provided herein are methods for improving the dispersion of an amino acid, e.g., a hydrophobic amino acid, in an aqueous suspension using a wetting agent. Uses of a wetting agent as described herein provide an improved approach to manufacturing of compositions capable of delivering large doses of amino acids in small volumes. Without wishing to be bound by theory, the present invention is directed, in part, to aqueous suspensions comprising wetting agents, and methods of providing and/or combining the same to produce aqueous suspensions that have high concentrations of amino acids (e.g., a hydrophobic amino acid), low volumes, and improved dispersion of said amino acids (e.g., a hydrophobic amino acid). Such aqueous suspensions may evenly distribute component amino acids, exhibiting characteristics such as improved mouthfeel, taste, appearance, preparation time (e.g., constitution time), and/or dose delivery. In some embodiments, an aqueous suspension has a volume less than or equal to 8, 7, 6, 5, 4, 3, 2, 1, or 0.5 ounces, and an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce or a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce. In some embodiments, a wetting agent for use in an aqueous suspension has an HLB value of either 8-9 or 2-3.

Accordingly, in one aspect, the invention features a method of improving the dispersion of a hydrophobic amino acid in an aqueous suspension, the method comprising providing a dry blended preparation comprising a hydrophobic amino acid entity, and a wetting agent; and combining the dry blended preparation, the wetting agent, and water to form an aqueous suspension that achieves a standard for suspension uniformity, thereby improving the dispersion of a hydrophobic amino acid in the aqueous suspension.

In another aspect, the invention features a method of manufacturing, making, or formulating an aqueous suspension, the method comprising: providing a dry blended preparation comprising at least one hydrophobic amino acid entity, and a wetting agent; and combining the dry blended preparation, the wetting agent, and water to form an aqueous suspension that achieves a standard for suspension uniformity, thereby manufacturing, making, or formulating an aqueous suspension.

In another aspect, the invention features a method of reducing foaming of an aqueous suspension comprising a hydrophobic amino acid, the method comprising: providing a dry blended preparation comprising at least one hydrophobic amino acid entity, and a wetting agent; and combining the dry blended preparation, the wetting agent, and water to form an aqueous suspension that achieves a standard for suspension uniformity, thereby reducing foaming of an aqueous suspension comprising a hydrophobic amino acid.

In another aspect, the invention features a method of increasing the solubilization rate of an amino acid (e.g., hydrophobic amino acid) in an aqueous suspension, the method comprising: providing a dry blended preparation comprising at least one hydrophobic amino acid entity, and a wetting agent; and combining the dry blended preparation, the wetting agent, and water to form an aqueous suspension that achieves a standard for suspension uniformity, thereby increasing the solubilization rate of an amino acid (e.g., hydrophobic amino acid) in an aqueous suspension.

In another aspect, the invention features an aqueous suspension comprising: a plurality of amino acid entities, including at least one hydrophobic amino acid entity; a wetting agent; and water, wherein the aqueous suspension achieves a standard for suspension uniformity.

In another aspect, the invention features a method of making a dry blended preparation with improved hydrophobic amino acid dispersion, the method comprising: providing a dry blended preparation comprising at least one hydrophobic amino acid entity and a wetting agent, wherein, when the dry blended preparation is combined with water to form an aqueous suspension the aqueous suspension achieves a standard for suspension uniformity, thereby making a dry blended preparation with improved hydrophobic amino acid dispersion.

In another aspect, the invention features a dry blended preparation comprising: a plurality of amino acid entities, including at least one hydrophobic amino acid entity; and a wetting agent, e.g., with an HLB value of 8-9 or 2-3, wherein, when the dry blended preparation is combined with water to form an aqueous suspension, the aqueous suspension achieves a standard for suspension uniformity.

In another aspect, the invention features a kit comprising: a plurality of amino acid entities, including at least one hydrophobic amino acid entity; a wetting agent; and instructions for combining the plurality of amino acid entities and the wetting agent into an aqueous suspension that achieves a standard for suspension uniformity.

In another aspect, the invention features a method of directing provision of an aqueous suspension, the method comprising: providing a dry blended preparation comprising at least one hydrophobic amino acid entity, and a wetting agent; and providing instructions for combining the dry blended preparation, the wetting agent, and water to form an aqueous suspension that achieves a standard for suspension uniformity, thereby directing provision of an aqueous suspension.

In another aspect, the invention features a method of evaluating the dispersion of a dry blended preparation comprising at least one hydrophobic amino acid entity, the method comprising: providing a dry blended preparation comprising at least one hydrophobic amino acid entity, and a wetting agent; combining the dry blended preparation, wetting agent, and water to form an aqueous suspension; and evaluating whether the aqueous suspension achieves a standard for suspension uniformity (e.g., as defined by a government regulatory agency or by standard industry practice) by acquiring a value for the amount of an amino acid entity (e.g., a hydrophobic amino acid entity) at a sampling point (e.g., in weight % of total amino acid entities or weight % of the total mixture), and optionally comparing said value to a reference value, thereby evaluating the dispersion of the dry blended preparation.

In some embodiments, the aqueous suspension has a volume less than or equal to 8, 7, 6, 5, 4, 3, 2, 1, or 0.5 ounces, and one or both of: i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce. In some embodiments, the wetting agent has an HLB value of either 8-9 or 2-3. In some embodiments, the wetting agent has an HLB value of either 8-9 or 2-3, and the aqueous suspension has a volume less than or equal to 8, 7, 6, 5, 4, 3, 2, 1, or 0.5 ounces, and one or both of: i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce.

In certain embodiments, provided herein is a dry blended composition including a) a leucine amino acid entity, b) an isoleucine amino acid entity, c) a valine amino acid entity, d) an arginine amino acid entity, e) a glutamine amino acid entity, and f) an N-acetylcysteine (NAC) entity, wherein the wetting agent is lecithin, e.g., a lecithin having an HLB value of 8-9 and/or 20 to 40% amphiphilic phospholipid (e.g., phosphatidylcholine), e.g., Alcolec 40P or a substantially equivalent lecithin.

In certain embodiments, provided herein is a dry blended composition including a) a leucine amino acid entity, b) an isoleucine amino acid entity, c) a valine amino acid entity, d) a histidine amino acid entity, e) a lysine amino acid entity, f) a threonine amino acid entity, g) an ornithine amino acid entity, and h) an aspartate amino acid entity, wherein the wetting agent is lecithin, e.g., a lecithin having an HLB value of 8-9 and/or 20 to 40% amphiphilic phospholipid (e.g., phosphatidylcholine), e.g., Alcolec 40P or a substantially equivalent lecithin.

In further embodiments, the dry blended composition includes one or more pharmecutically acceptable excipients. In further embodiments, the dry blended composition includes an adsorbent, e.g., SiO₂.

Provided herein are methods of stabilizing N-acetylcysteine (NAC) in the presence of an acetyl acceptor, e.g., carnitine (CAR), comprising forming a dry blended preparation comprising NAC and the acetyl acceptor (e.g., CAR) under conditions such that deacetylation of NAC in the dry blended preparation is decreased compared to the deacetylation in a reference mixture (e.g., a dry blended preparation not formed under the conditions that decrease deacetylation of NAC). In some embodiments, the conditions comprise including an adsorbent in the dry blended preparation. Without wishing to be bound by theory, an adsorbent may bind the water present in a dry blended preparation, e.g., preventing the water from facilitating the deacetylation of NAC, e.g., by CAR or water associated with CAR. A dry blended preparation formed under conditions that decrease deacetylation of NAC may have improved stability and longevity (e.g., shelf life) compared to dry blended preparations comprising NAC and not formed under conditions decreasing deacetylation.

Accordingly, in one aspect, the invention features a method of stabilizing NAC, e.g., in the presence of an acetyl acceptor, e.g., CAR, the method comprising: forming a dry blended preparation comprising NAC and CAR, under conditions such that deacetylation of NAC in the dry blended preparation is decreased compared to the deacetylation in a reference mixture, wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, thereby stabilizing NAC.

In another aspect, the invention features a method of decreasing the deacetylation of NAC in the presence of an acetyl acceptor (e.g., (CAR)), the method comprising: forming a dry blended preparation comprising NAC and CAR, under conditions such that deacetylation of NAC in the dry blended preparation is decreased compared to the deacetylation in a reference mixture, wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, thereby decreasing the deacetylation of NAC in the presence of an acetyl acceptor (e.g., CAR).

In another aspect, the invention features a method of making a dry blended preparation comprising NAC and an acetyl acceptor, e.g., (CAR), the method comprising: forming a dry blended preparation comprising NAC and the acetyl acceptor, e.g. CAR, under conditions such that deacetylation of NAC in the dry blended preparation is decreased compared to the deacetylation in a reference mixture, wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, thereby making a dry blended preparation comprising NAC and an acetyl acceptor (e.g., CAR).

In another aspect, the invention features a method of evaluating the stability of NAC in a dry blended preparation, the method comprising: providing a dry blended preparation comprising NAC; and determining the level of NAC, e.g., at a first time point and at a second time point; and optionally, comparing the level of NAC to a reference value, e.g., a NAC value between 90-110% of the original level of NAC in the mixture, wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, thereby evaluating the stability of NAC in a dry blended preparation.

In another aspect, the invention features a method of pre-treating NAC, the method comprising: providing a mixture of NAC and an adsorbent, thereby pre-treating NAC.

In another aspect, the invention features a method of pre-treating CAR, the method comprising: providing a mixture of CAR and an adsorbent, thereby pre-treating CAR.

In another aspect, the invention features a dry blended preparation comprising: NAC; an acetyl acceptor (e.g., CAR); and an adsorbent (e.g., SiO₂), wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, and wherein the adsorbent (e.g., SiO₂) is present at a weight percentage (w/w) of at least 0.05, 0.075, 0.1, 0.125, 0.15, 0.175, 0.2, 0.225, 0.25, 0.275, 0.3, 0.325, 0.35, 0.4, 0.425, 0.45, 0.475, 0.5, 0.6, 0.7, 0.8, 0.9, 1, 1.5, 2, 2.5, 3, 3.5, 4, 4.5, or 5%, e.g., at least 0.3%, (and optionally at less than 10, 9, 8, 7, 6, 5, 4, 3, 2, 1.5, 1, 0.9, 0.8, 0.7, 0.6, 0.575, 0.55, 0.525, 0.5, 0.475, 0.45, 0.425, 0.4, 0.375, 0.35, or 0.325%).

In another aspect, the invention features a mixture, e.g., a dry blended preparation, comprising: NAC and an adsorbent (e.g., SiO₂), wherein the mixture, e.g., dry blended preparation, comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, and wherein the adsorbent (e.g., SiO₂) is present at a weight percentage (w/w) of at least 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, or 10% (and optionally at less than 20, 19, 18, 17, 16, 15, 14, 13, 12, 11, 10, 9, 8, 7, or 6.5%). In some embodiments, the mixture comprises no other amino acid entity besides NAC. In some embodiments, the mixture is a dry blended preparation. In some embodiments, the mixture, e.g., dry blended preparation, does not comprise CAR.

In further embodiments, the dry blended preparation may include: a) a leucine (L)-amino acid entity, b) an arginine (R)-amino acid entity, and/or c) a glutamine (Q)-amino acid entity.

In further embodiments, the dry blended preparation may include: a) a leucine (L)-amino acid entity, b) an arginine (R)-amino acid entity, and c) a glutamine (Q)-amino acid entity.

In further embodiments, the dry blended preparation may include: a) a leucine (L)-amino acid entity, b) an arginine (R)-amino acid entity, c) a glutamine (Q)-amino acid entity, d) a citrulline amino acid entity, e) a serine (S)-amino acid entity, f) a valine (V)-amino acid entity, g) a histidine (H)-amino acid entity, and h) a lysine (K)-amino acid entity.

In further embodiments, the dry blended preparation may include: a) a leucine (L)-amino acid entity, b) an arginine (R)-amino acid entity, c) a glutamine (Q)-amino acid entity, d) an isoleucine (I)-amino acid entity, and e) a serine (S)-amino acid entity.

Additional features of any of the aforesaid methods or compositions include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

All publications, patent applications, patents, and other references (e.g., sequence database reference numbers) mentioned herein are incorporated by reference in their entirety.

### ENUMERATED EMBODIMENTS

1. A method of improving the dispersion of a hydrophobic amino acid in an aqueous suspension, the method comprising:
   providing a dry blended preparation comprising a hydrophobic amino acid entity, and a wetting agent; and
   combining the dry blended preparation, the wetting agent, and water to form an aqueous suspension that achieves a standard for suspension uniformity,
   wherein the aqueous suspension has a volume less than or equal to 8, 7, 6, 5, 4, 3, 2, 1, or 0.5 ounces, and one or both of:
      i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
      ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce,
   thereby improving the dispersion of a hydrophobic amino acid in the aqueous suspension.
2. A method of improving the dispersion of a hydrophobic amino acid in an aqueous suspension, the method comprising:
   providing a dry blended preparation comprising at least one hydrophobic amino acid entity, and a wetting agent with an HLB value of either 8-9 or 2-3; and
   combining the dry blended preparation, the wetting agent, and water to form an aqueous suspension that achieves a standard for suspension uniformity,
   thereby improving the dispersion of a hydrophobic amino acid in an aqueous suspension.
3. A method of manufacturing, making, or formulating an aqueous suspension, the method comprising:
   providing a dry blended preparation comprising at least one hydrophobic amino acid entity, and a wetting agent; and
   combining the dry blended preparation, the wetting agent, and water to form an aqueous suspension that achieves a standard for suspension uniformity,
   wherein the aqueous suspension has a volume less than or equal to 8, 7, 6, 5, 4, 3, 2, 1, or 0.5 ounces, and one or both of:
      i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
      ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce,
   thereby manufacturing, making, or formulating an aqueous suspension.
4. A method of manufacturing, making, or formulating an aqueous suspension, the method comprising:
   providing a dry blended preparation comprising at least one hydrophobic amino acid entity, and a wetting agent with an HLB value of either 8-9 or 2-3; and
   combining the dry blended preparation, the wetting agent, and water to form an aqueous suspension that achieves a standard for suspension uniformity,
   thereby manufacturing, making, or formulating an aqueous suspension.
5. A method of reducing foaming of an aqueous suspension comprising a hydrophobic amino acid, the method comprising:
   providing a dry blended preparation comprising at least one hydrophobic amino acid entity, and a wetting agent; and
   combining the dry blended preparation, the wetting agent, and water to form an aqueous suspension that achieves a standard for suspension uniformity,
   wherein the aqueous suspension has a volume less than or equal to 8, 7, 6, 5, 4, 3, 2, 1, or 0.5 ounces, and one or both of:
      i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
      ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce,
   thereby reducing foaming of an aqueous suspension comprising a hydrophobic amino acid.
6. A method of reducing foaming of an aqueous suspension comprising a hydrophobic amino acid, the method comprising:
   providing a dry blended preparation comprising at least one hydrophobic amino acid entity, and a wetting agent with an HLB value of either 8-9 or 2-3; and
   combining the dry blended preparation, the wetting agent, and water to form an aqueous suspension that achieves a standard for suspension uniformity,
   thereby reducing foaming of an aqueous suspension comprising a hydrophobic amino acid.
7. A method of increasing the solubilization rate of an amino acid (e.g., hydrophobic amino acid) in an aqueous suspension, the method comprising:
   providing a dry blended preparation comprising at least one hydrophobic amino acid entity, and a wetting agent; and
   combining the dry blended preparation, the wetting agent, and water to form an aqueous suspension that achieves a standard for suspension uniformity,
   wherein the aqueous suspension has a volume less than or equal to 8, 7, 6, 5, 4, 3, 2, 1, or 0.5 ounces, and one or both of:
      i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
      ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce,
   thereby increasing the solubilization rate of an amino acid (e.g., hydrophobic amino acid) in an aqueous suspension. 8. A method of increasing the solubilization rate of an amino acid (e.g., hydrophobic amino acid) in an aqueous suspension, the method comprising:
   providing a dry blended preparation comprising at least one hydrophobic amino acid entity, and a wetting agent with an HLB value of either 8-9 or 2-3; and
   combining the dry blended preparation, the wetting agent, and water to form an aqueous suspension that achieves a standard for suspension uniformity,
   thereby increasing the solubilization rate of an amino acid (e.g., hydrophobic amino acid) in an aqueous suspension. 9. An aqueous suspension comprising:
   a plurality of amino acid entities, including at least one hydrophobic amino acid entity;
   a wetting agent; and
   water,
   wherein the aqueous suspension achieves a standard for suspension uniformity, and
   wherein the aqueous suspension has a volume less than or equal to 8, 7, 6, 5, 4, 3, 2, 1, or 0.5 ounces, and one or both of:
      i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
      ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce.
10. An aqueous suspension comprising:
   a plurality of amino acid entities, including at least one hydrophobic amino acid entity;
   a wetting agent with an HLB value of either 8-9 or 2-3; and
   water,
   wherein the aqueous suspension achieves a standard for suspension uniformity.
11. A method of making a dry blended preparation with improved hydrophobic amino acid dispersion, the method comprising:
   providing a dry blended preparation comprising at least one hydrophobic amino acid entity and a wetting agent,
   wherein, when the dry blended preparation is combined with water to form an aqueous suspension with volume less than or equal to 8, 7, 6, 5, 4, 3, 2, 1, or 0.5 ounces, and one or both of:
      i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
      ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce,
the aqueous suspension achieves a standard for suspension uniformity,
   thereby making a dry blended preparation with improved hydrophobic amino acid dispersion.
12. A method of making a dry blended preparation with improved hydrophobic amino acid dispersion, the method comprising:
   providing a dry blended preparation comprising at least one hydrophobic amino acid entity and a wetting agent with an HLB value of either 8-9 or 2-3,
   wherein, when the dry blended preparation is combined with water to form an aqueous suspension, the aqueous suspension achieves a standard for suspension uniformity,
   thereby making a dry blended preparation with improved hydrophobic amino acid dispersion.
13. A dry blended preparation comprising:
   a plurality of amino acid entities, including at least one hydrophobic amino acid entity; and
   a wetting agent with an HLB value of 8-9 or 2-3,
   wherein, when the dry blended preparation is combined with water to form an aqueous suspension, the aqueous suspension achieves a standard for suspension uniformity.
14. A dry blended preparation comprising:
   a plurality of amino acid entities, including at least one hydrophobic amino acid entity; and
   a wetting agent with an HLB value of 8-9 or 2-3,
   wherein, when the dry blended preparation is combined with water to form an aqueous suspension with volume less than or equal to 8, 7, 6, 5, 4, 3, 2, 1, or 0.5 ounces, and one or both of:
      i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
      ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce,
the aqueous suspension achieves a standard for suspension uniformity
15. A kit comprising:
   a plurality of amino acid entities, including at least one hydrophobic amino acid entity;
   a wetting agent; and
   instructions for combining the plurality of amino acid entities and the wetting agent into an aqueous suspension that achieves a standard for suspension uniformity,
   wherein the aqueous suspension has a volume less than or equal to 8, 7, 6, 5, 4, 3, 2, 1, or 0.5 ounces, and one or both of:
      i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15g/ounce; or
      ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce.
16. A kit comprising:
   a plurality of amino acid entities, including at least one hydrophobic amino acid entity;
   a wetting agent with an HLB value of either 8-9 or 2-3; and
   instructions for combining the plurality of amino acid entities and the wetting agent into an aqueous suspension that achieves a standard for suspension uniformity.
17. A method of directing provision of an aqueous suspension, the method comprising:
   providing a dry blended preparation comprising at least one hydrophobic amino acid entity, and a wetting agent; and
   providing instructions for combining the dry blended preparation, the wetting agent, and water to form an aqueous suspension that achieves a standard for suspension uniformity,
   wherein the aqueous suspension has a volume less than or equal to 8, 7, 6, 5, 4, 3, 2, 1, or 0.5 ounces, and one or both of:
      i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15g/ounce; or
      ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce,
   thereby directing provision of an aqueous suspension.
18. A method of directing provision of an aqueous suspension, the method comprising:
   providing a dry blended preparation comprising at least one hydrophobic amino acid entity, and a wetting agent with an HLB value of either 8-9 or 2-3; and
   providing instructions for combining the dry blended preparation, the wetting agent, and water to form an aqueous suspension that achieves a standard for suspension uniformity,
   thereby directing provision of an aqueous suspension.
19. A method of evaluating the dispersion of a dry blended preparation comprising at least one hydrophobic amino acid entity, the method comprising:
   providing a dry blended preparation comprising at least one hydrophobic amino acid entity, and a wetting agent;
   combining the dry blended preparation, wetting agent, and water to form an aqueous suspension; and
   evaluating whether the aqueous suspension achieves a standard for suspension uniformity (e.g., as defined by a government regulatory agency or by standard industry practice) by acquiring a value for the amount of an amino acid entity (e.g., a hydrophobic amino acid entity) at a sampling point (e.g., in weight % of total amino acid entities or weight % of the total mixture), and optionally comparing said value to a reference value,
   thereby evaluating the dispersion of the dry blended preparation.
20. The method, aqueous suspension, dry blended preparation, or kit of any of embodiments 1-19, wherein the wetting agent has an HLB value of 8.
21. The method, aqueous suspension, dry blended preparation, or kit of any of embodiments 1-19, wherein the wetting agent has an HLB value of 9.
22. The method, aqueous suspension, dry blended preparation, or kit of any of embodiments 1-19, wherein the wetting agent has an HLB value of 2.
23. The method, aqueous suspension, dry blended preparation, or kit of any of embodiments 1-19, wherein the wetting agent has an HLB value of 3.
24. The method, aqueous suspension, dry blended preparation, or kit of any of embodiments 1-23, wherein the wetting agent modulates (e.g., decreases or increases) the surface tension of the aqueous suspension to a range of surface tension values where efficient wetting of the hydrophobic amino acids is achieved (e.g., as measured by force tensiometer, optical tensiometer, or contact angle).
25. The method, aqueous suspension, dry blended preparation, or kit of any of embodiments 1-24, wherein the wetting agent is a surfactant that decreases the surface tension of the aqueous suspension (e.g., as measured by force tensiometer, optical tensiometer, or contact angle).
26. The method, aqueous suspension, dry blended preparation, or kit of any of embodiments 24 or 25, wherein the range of surface tension values is 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 70-90, 70-100, 80-90, 80-100, or 90-100 millinewtons per meter (mN/m) (e.g., as measured by force tensiometer, optical tensiometer, or contact angle).
27. The method, aqueous suspension, dry blended preparation, or kit of any of embodiments 1-26, wherein the wetting agent is chosen from a lecithin or a poloxamer.
28. The method, aqueous suspension, dry blended preparation, or kit of any of embodiments 1-27, wherein the wetting agent is chosen from:
   i) lecithin 40P or a substantially equivalent lecithin,
   ii) Lipoid 20S or a substantially equivalent lecithin, or
   iii) poloxamer P331 or a substantially equivalent poloxamer.
29. The method, aqueous suspension, dry blended preparation, or kit of any of embodiments 1-28, wherein the wetting agent is lecithin 40P or a substantially equivalent lecithin.
30. The method, aqueous suspension, dry blended preparation, or kit of any of embodiments 1-28, wherein the wetting agent is Lipoid 20S or a substantially equivalent lecithin.
31. The method, aqueous suspension, dry blended preparation, or kit of any of embodiments 1-28, wherein the wetting agent is poloxamer P331 or a substantially equivalent poloxamer.
32. The method, aqueous suspension, dry blended preparation, or kit of any of embodiments 1-31, wherein the wetting agent has a Hydrophilic-Lipophilic Balance (HLB) of 2-3, 7-9, or 8-9, e.g., 2-3 or 8-9.
33. The method, amino acid mixture, dry blended preparation, or kit of any of embodiments 1-30 or 32, wherein the wetting agent is a lecithin comprising one, two, or all of:
   a) at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% phosphatidylcholine (PC) (and optionally less than 50, 45, or 40% phosphatidylcholine (PC));
   b) at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% phosphatidylinositol (and optionally less than 50, 40, 30, 20, 19, 18, 17, 16, or 15% phosphatidylinositol); and
   a) at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% phosphatidylethanolamine (and optionally less than 50, 45, or 40% phosphatidylethanolamine).
34. The method, amino acid mixture, dry blended preparation, or kit of any of embodiments 1-30, 32, or 33, wherein the wetting agent is a lecithin comprising at least about 20 to 40% amphiphilic phospholipid (e.g., phosphatidylcholine (PC)).
35. The method, amino acid mixture, dry blended preparation, or kit of any of embodiments 1-28 or 32-34, wherein the wetting agent is Alcolec Lecithin 40P or a lecithin substantially equivalent thereto.
36. The method, amino acid mixture, dry blended preparation, or kit of any of embodiments 1-28, 32, or 33, wherein the wetting agent is Lipoid 20S Lecithin or a lecithin substantially equivalent thereto.
37. The method, amino acid mixture, dry blended preparation, or kit of any of embodiments 1-28, 31, or 32, wherein the wetting agent is a poloxamer with a polyoxypropylene core molecular mass of at least 2500, 2600, 2700, 2800, 2900, 3000, 3100, 3200, or 3300 g/mol (and optionally no more than 4000, 3900, 3800, 3700, 3600, 3500, 3400, or 3300 g/mol).
38. The method, amino acid mixture, dry blended preparation, or kit of any of embodiments 1-28, 31, 32, or 37, wherein the wetting agent is a poloxamer with a percentage polyoxyethylene content of at least 1, 2, 3, 4, 5, 6, 7, 8, 9, or 10% (and optionally, no more than 20, 19, 18, 17, 16, 15, 14, 13, 12, 11, or 10%).
39. The method, amino acid mixture, dry blended preparation, or kit of any of embodiments 1-28, 31, 32, 37, or 38, wherein the wetting agent is a poloxamer has a polyoxypropylene core molecular mass of about 3300 g/mol (e.g., 3300 g/mol) and a percentage polyoxyethylene content of about 10% (e.g., 10%).
40. The method, amino acid mixture, dry blended preparation, or kit of any of embodiments 1-28, 31, 32, or 37-39, wherein the wetting agent is a P331 poloxamer or poloxamer substantially equivalent thereto.
41. The method, aqueous suspension, dry blended preparation, or kit of any of embodiments 1-40, wherein the dry blended preparation comprises the wetting agent.
42. The method, aqueous suspension, dry blended preparation, or kit of any of embodiments 1-40, wherein the dry blended preparation does not comprise the wetting agent (e.g., until during or after the combining step).
43. The method, aqueous suspension, dry blended preparation, or kit of any of embodiments 1-40 or 42, wherein the wetting agent is provided in the water used to form the aqueous suspension.
44. The method, aqueous suspension, dry blended preparation, or kit of any of embodiments 1-43, wherein the aqueous suspension and/or dry blended preparation comprises one or more excipients in addition to the wetting agent.
45. The method, aqueous suspension, dry blended preparation, or kit of any of embodiments 1-44, wherein the aqueous suspension and/or dry blended preparation does not comprise any additional wetting agent.
46. The method, aqueous suspension, dry blended preparation, or kit of any of embodiments 1-45, wherein the aqueous suspension has a volume of about 2 ounces per dose or per dosing period.
47. The method, aqueous suspension, dry blended preparation, or kit of any of embodiments 1-45, wherein the aqueous suspension has a volume of about 1 ounce per dose or per dosing period.
48. The method, aqueous suspension, dry blended preparation, or kit of any of embodiments 1-45, wherein the aqueous suspension has a volume of about 0.5 ounces per dose or per dosing period.
49. The method, aqueous suspension, dry blended preparation, or kit of any of embodiments 1-45, wherein the aqueous suspension has a volume of about 0.33 ounces per dose or per dosing period.
50. The method, aqueous suspension, dry blended preparation, or kit of any of embodiments 1-49, wherein the wetting agent, e.g., lecithin or poloxamer, is 0.5-4% (w/w) of the dry mixture or 0.1-1.5% (w/w) of the aqueous suspension.
51. The method, aqueous suspension, dry blended preparation, or kit of any of embodiments 1-49, wherein the wetting agent, e.g., lecithin, is at least 0.5, 0.55, 0.6, 0.65, 0.7, 0.75, 0.8, 0.85, 0.9, 0.95, 1, or 1.05% (w/w) of the dry weight of the combination of the plurality of amino acid entities, wetting agent, and any other excipient (and optionally less than or equal to 5, 4, 3, 2, 1.9, 1.8, 1.7, 1.6, 1.5, 1.4, 1.3, 1.2, or 1.1%).
52. The method, aqueous suspension, dry blended preparation, or kit of any of embodiments 1-49 or 51, wherein the wetting agent, e.g., lecithin, is no more than 5, 4, 3, 2, 1.9, 1.8, 1.7, 1.6, 1.5, 1.4, 1.3, 1.2, or 1.1% (w/w) of the dry weight of the combination of the plurality of amino acid entities, wetting agent, and any other excipient (and optionally at least 0.5, 0.55, 0.6, 0.65, 0.7, 0.75, 0.8, 0.85, 0.9, 0.95, 1, or 1.05%).
53. The method, aqueous suspension, dry blended preparation, or kit of any of embodiments 1-49, wherein the wetting agent, e.g., lecithin, is about 0.5-1.5% (w/w) of the dry weight of the combination of the plurality of amino acid entities, wetting agent, and any other excipient.
54. The method, aqueous suspension, dry blended preparation, or kit of any of embodiments 1-49, wherein the wetting agent, e.g., poloxamer, is at least 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, or 2.5% (w/w) of the dry weight of the combination of the plurality of amino acid entities, wetting agent, and any other excipient (and optionally less than or equal to 5, 4, 3, 2, 1.9, 1.8, 1.7, 1.6, 1.5, 1.4, 1.3, 1.2, 1.1, 1, 0.9, 0.8, 0.7, 0.6, or 0.5% (w/w)).
55. The method, aqueous suspension, dry blended preparation, or kit of any of embodiments 1-49 or 54, wherein the wetting agent, e.g., poloxamer, is no more than 5, 4, 3, 2, 1.9, 1.8, 1.7, 1.6, 1.5, 1.4, 1.3, 1.2, 1.1, 1, 0.9, 0.8, 0.7, 0.6, or 0.5% (w/w) of the dry weight of the combination of the plurality of amino acid entities, wetting agent, and any other excipient (and optionally at least 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, or 2.5% (w/w)).
56. The method, aqueous suspension, dry blended preparation, or kit of any of embodiments 1-49, 54, or 55, wherein the wetting agent, e.g., poloxamer, is about 0.5-2.25% (w/w) of the dry weight of the combination of the plurality of amino acid entities, wetting agent, and any other excipient.
57. The method, dry blended preparation, or kit of any of embodiments 1-8 or 11-56, wherein combining comprises shaking or inverting the dry blended preparation, the wetting agent, and water (e.g., in a container, e.g., jar).
58. The method, dry blended preparation, or kit of embodiment 57, wherein shaking or inverting the dry blended preparation, the wetting agent, and water has a duration of at least 10, 20, 30, 40, 50, or 60 seconds, or 1, 2, 3, 4, 5, 6, 7, 8, 9, 10, 15, 20, 25, 30, 35, 40, 45, 50, 55, or 60 minutes (and optionally, no more than 120, 100, 80, 60, 40, 30, or 15 minutes, or 60, 50, 40, or 30 seconds).
59. The method, dry blended preparation, or kit of embodiment 57, wherein shaking or inverting the dry blended preparation, the wetting agent, and water continues until the aqueous suspension appears uniform, e.g., by visual inspection.
60. The method, dry blended preparation, or kit of embodiment 57, wherein shaking or inverting the dry blended preparation, the wetting agent, and water continues until the aqueous suspension achieves a standard for suspension uniformity.
61. The method, dry blended preparation, or kit of any of embodiments 1-8 or 11-56, wherein combining comprises subjecting the plurality of amino acid entities, the wetting agent, and water to a blending condition, e.g., that uses convection diffusion, or shear blending mechanism.
62. The method, aqueous suspension, dry blended preparation, or kit of any of embodiments 1-61, wherein the dry blended preparation and/or aqueous suspension is suitable for oral administration.
63. The method, aqueous suspension, dry blended preparation, or kit of any of embodiments 1-62 wherein the dry blended preparation and/or aqueous suspension further comprises one or more excipients.
64. The method, aqueous suspension, dry blended preparation, or kit of embodiment 63, wherein the excipient is an excipient that is suitable for oral administration.
65. The method, aqueous suspension, dry blended preparation, or kit of either embodiment 63 or 64, wherein the excipient is a flavoring agent.
66. The method, aqueous suspension, dry blended preparation, or kit of embodiment 65, wherein the flavoring agent produces a flavor selected from peach, mango, lemon, lime, orange, and orange creamsicle.
66. The method, aqueous suspension, dry blended preparation, or kit of embodiment 66, wherein the flavor is orange creamsicle.
67. The method, aqueous suspension, dry blended preparation, or kit of any of embodiments 63-66, wherein the excipient is a substance generally regarded as safe (GRAS), e.g., a substance from the Federal Drug Administration's GRAS Notice List.
68. The method, aqueous suspension, dry blended preparation, or kit of any of embodiments 1-67, wherein the dry blended preparation and/or aqueous suspension is not sufficiently sterile for parenteral administration.
69. The method, aqueous suspension, dry blended preparation, or kit of any of embodiments 1-68, wherein the level of microbial contamination of the dry blended preparation and/or aqueous suspension is below the level permitted in food.
70. The method, aqueous suspension, dry blended preparation, or kit of any of embodiments 1-69, wherein the dry blended preparation meets a reference standard, e.g., composition uniformity.
71. The method, aqueous suspension, dry blended preparation, or kit of embodiment 70, wherein the reference standard is set by a manufacturer of the dry blended preparation.
72. The method, aqueous suspension, dry blended preparation, or kit of embodiment 70, wherein the reference standard is set by a manufacturer having approval from a governmental agency to market the dry blended preparation.
73. The method, aqueous suspension, dry blended preparation, or kit of embodiment 70, wherein the reference standard is set by a pharmacopeal entity, or a national formulary, or found in a pharmacopeal reference, e.g., the USP or NF.
74. The method, aqueous suspension, dry blended preparation, or kit of embodiment 70, wherein the reference standard is set by a a governmental agency, e.g., a government agency that authorizes or regulates the manufacture or marketing of the dry blended preparation.
75. The method, aqueous suspension, dry blended preparation, or kit of embodiment 74, wherein the government agency comprises one or more of the following agencies: Federal Drug Administration (FDA), European Medicines Agency (EMA), SwissMedic, China Food and Drug Administration (CFDA), Japanese Pharmaceuticals and Medical Devices Agency (PMDA), Health Canada, or Medicines and Healthcare Products Regulatory Agency (MHRA).
76. The method, dry blended preparation, or kit of any of embodiments 1-8 or 11-75, wherein combining produces a layer of foam at the top of the aqueous suspension.
77. The method, dry blended preparation, or kit of embodiment 76, wherein combining comprises holding the aqueous suspension, e.g., after shaking or inverting or subjecting the aqueous suspension to a blending condition (e.g., to reduce the size of the layer of foam).
78. The method, dry blended preparation, or kit of embodiment 77, wherein holding the aqueous suspension reduces the size of the layer of foam such that the layer of foam does not interfere with downstream processing or use (e.g., imbibing) by an end user of the aqueous suspension.
79. The method, dry blended preparation, or kit of embodiment 77, wherein holding the aqueous suspension reduces the thickness of the layer of foam by at least 10, 20, 30, 40, 50, 60, 70, 80, 90, or 100% (e.g., eliminates the layer of foam).
80. The method, dry blended preparation, or kit of embodiment 77, wherein holding the aqueous suspension has a duration of less than 20, 19, 18, 17, 16, 15, 14, 13, 12, 11, 10, 9, 8, 7, 6, 5, 4, or 3 minutes, e.g., less than 10 or 5 minutes, (and optionally at least 10, 20, 30, 40, 50, or 60 seconds, or at least 1, 2, 3, 4, or 5 minutes).
81. The method, dry blended preparation, or kit of any of embodiments 77-80, wherein holding continues until the aqueous suspension achieves a standard for suspension uniformity.
82. The method, aqueous suspension, dry blended preparation, or kit of 1-81, wherein the standard for suspension uniformity comprises the amount of an amino acid entity (e.g., a hydrophobic amino acid entity) at a sampling point (e.g. in weight % of total amino acid entities or weight % of the total mixture) differing from the amount of the amino acid entity (e.g., a hydrophobic amino acid entity) present in the aqueous suspension by less than a predetermined amount, e.g., less than 10, 9, 8, 7, 6, 5, 4, 3, 2, or 1%.
82. The method, aqueous suspension, dry blended preparation, or kit of 1-81, wherein the standard for suspension uniformity comprises the amount of an amino acid entity (e.g., a hydrophobic amino acid entity) at a sampling point (e.g. in weight % of total amino acid entities or weight % of the total mixture) differing from the amount of the amino acid entity (e.g., a hydrophobic amino acid entity) present at a second sampling point by less than a predetermined amount, e.g., less than 10, 9, 8, 7, 6, 5, 4, 3, 2, or 1%.
83. The method of any of 1-8, 11, 12, or 17-82, comprising acquiring a value for the amount of an amino acid entity (e.g., a hydrophobic amino acid entity) at a sampling point (e.g. in weight % of total amino acid entities or weight % of the total mixture).
84. The method of embodiment 83, comprising comparing the value to a reference value, e.g., the amount of the amino acid entity (e.g., a hydrophobic amino acid entity) present in the aqueous suspension.
85. The method of any of 1-8, 11, 12, or 17-84, comprising acquiring a value for the amount of an amino acid entity (e.g., a hydrophobic amino acid entity) at a sampling point (e.g. in weight % of total amino acid entities or weight % of the total mixture) and acquiring a value for the amount of the amino acid entity at a second sampling point.
86. The method of embodiment 85, comprising comparing the values.
87. The method of any of embodiments 83-86, wherein acquiring a value comprises using HPLC-UV, UPLC-UV, OPA tagged HPLC-UV, Accqtag HPLC-UV, and/or LC/MS.
88. The method of any of embodiments 83-87, wherein acquiring a value further comprises derivatizing one or more of the pharmaceutical amino acid entities with a detectable moiety.
89. The method of embodiment 88, wherein the detectable moiety is a fluorescent moiety.
90. The method of embodiment 89, wherein the fluorescent moiety is o-phthalaldehyde (OPA) or fluorenylmethoxy chloroformate (FMOC).
91. The method of embodiment 88, wherein the detectable moiety is a chromophore.
92. The method, dry blended preparation, or kit of any of embodiments 76-91, wherein the layer of foam has a smaller thickness relative to a layer of foam formed by combining a similar aqueous suspension not comprising the wetting agent.
93. The method, dry blended preparation, or kit of embodiment 92, wherein the layer of foam is at least 10, 20, 30, 40, 50, 60, 70, 80, 90, or 100% less thick than the layer of foam formed by combining a similar aqueous suspension not comprising the wetting agent.
94. The method, dry blended preparation, or kit of any of embodiments 76-93, wherein holding the aqueous suspension reduces the size of the layer of foam faster than holding reduces the size of the layer of foam formed by combining a similar aqueous suspension not comprising the wetting agent.
95. The method, dry blended preparation, or kit of embodiment 94, wherein holding reduces the size of the layer of foam at least 10, 20, 30, 40, 50, 60, 70, 80, 90, 100, 150, 200, 300, 400, 500, or 1000% faster than holding reduces the size of the layer of foam formed by combining a similar aqueous suspension not comprising the wetting agent.
96. The method, dry blended preparation, or kit of any of embodiments 1-8 or 11-95, wherein combining, e.g., constitution and/or reconstitution, does not form a layer of foam.
97. The method, aqueous suspension, dry blended preparation, or kit of any of embodiments 1-96, wherein the solubilization rate of an amino acid of the aqueous suspension (e.g., a hydrophobic amino acid) is increased relative to a similar suspension not comprising the wetting agent.
98. The method of embodiment 97, wherein the solubilization rate increases by at least 10, 20, 30, 40, 50, 60, 70, 80, 90, 100, 150, 200, 300, 400, 500, or 1000% relative to a similar suspension not comprising the wetting agent.
99. The method, aqueous suspension, dry blended preparation, or kit of any of embodiments 1-98, wherein the hydrophobic amino acid entity is a branched chain amino acid (BCAA).
100. The method, aqueous suspension, dry blended preparation, or kit of any of embodiments 1-99, wherein the hydrophobic amino acid entity is chosen from:
   a leucine amino acid entity;
   a valine amino acid entity;
   an isoleucine amino acid entity; or
   a tryptophan amino acid entity.
101. The method, aqueous suspension, dry blended preparation, or kit of any of embodiments 1-100, wherein the plurality of amino acid entities comprises one, two, three, four, or all of:
   a leucine amino acid entity;
   a valine amino acid entity;
   an isoleucine amino acid entity; or
   a tryptophan amino acid entity.
102. The method, aqueous suspension, dry blended preparation, or kit of any of embodiments 1-101, wherein the plurality of amino acid entities comprises:
   a leucine amino acid entity;
   an arginine amino acid entity;
   a glutamine amino acid entity; and
   an N-acetylcysteine (NAC) entity.
103. The method, aqueous suspension, dry blended preparation, or kit of any of embodiments 1-100, wherein the plurality of amino acid entities comprises:
   a leucine amino acid entity;
   an isoleucine amino acid entity;
   a valine amino acid entity;
   an arginine amino acid entity;
   a glutamine amino acid entity; and
   an N-acetylcysteine (NAC) entity.
104. The method, aqueous suspension, dry blended preparation, or kit of either of embodiments 102 or 103, wherein the plurality of amino acid entities comprises:
   a glycine amino acid entity; or
   a serine amino acid entity.
105. The method, aqueous suspension, dry blended preparation, or kit of either of embodiments 102 or 103, wherein the plurality of amino acid entities comprises one or more of:
   a histidine amino acid entity;
   a lysine amino acid entity;
   a phenylalanine amino acid entity; or
   a threonine amino acid entity.
106. The method, aqueous suspension, dry blended preparation, or kit of embodiment 102, wherein the plurality of amino acid entities comprises one or both of:
   a serine amino acid entity; and
   a carnitine amino acid entity.
107. The method, aqueous suspension, dry blended preparation, or kit of any of embodiments 1-100, wherein the plurality of amino acid entities comprises one, two, three, or all of:
   a leucine amino acid entity;
   an isoleucine amino acid entity;
   an arginine amino acid entity;
   an N-acetylcysteine (NAC) entity; or
   a carnitine amino acid entity.
108. The method, aqueous suspension, dry blended preparation, or kit of any of embodiments 1-100, wherein the plurality of amino acid entities comprises:
   one, two, or all of:
      a leucine amino acid entity;
      an isoleucine amino acid entity; or
      a valine amino acid entity;
   one, two, or all of:
      a histidine amino acid entity;
      a lysine amino acid entity; or
      a threonine amino acid entity; and
   one, two, or all of:
      an ornithine amino acid entity;
      an aspartate amino acid entity; or
      a combination salt of an ornithine amino acid entity and an aspartate amino acid entity.
109. The method, aqueous suspension, dry blended preparation, or kit of any of embodiments 1-100, wherein the plurality of amino acid entities comprises:
   an arginine amino acid entity;
   a citrulline amino acid entity; and
   an N-acetylcysteine (NAC) entity.
110. The method, aqueous suspension, dry blended preparation, or kit of embodiment 109, wherein the plurality of amino acid entities comprises:
   a glutamine amino acid entity; and
   a carnitine amino acid entity
111. The method, aqueous suspension, dry blended preparation, or kit of any of embodiments 1-100, wherein the plurality of amino acid entities comprises:
   one, two, or all of:
      a leucine amino acid entity;
      an isoleucine amino acid entity; or
      a valine amino acid entity;
   an N-acetylcysteine (NAC) entity; and
   an acetyl-carnitine (ALCAR) entity.
112. A method of stabilizing N-acetylcysteine (NAC), e.g., in the presence of carnitine (CAR), the method comprising:
   forming a dry blended preparation comprising NAC and CAR, under conditions such that deacetylation of NAC in the dry blended preparation is decreased compared to the deacetylation in a reference mixture,
   wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water,
   thereby stabilizing NAC.
113. A method of decreasing the deacetylation of N-acetylcysteine (NAC) in the presence of carnitine (CAR), the method comprising:
   forming a dry blended preparation comprising NAC and CAR, under conditions such that deacetylation of NAC in the dry blended preparation is decreased compared to the deacetylation in a reference mixture,
   wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water,
   thereby decreasing the deacetylation of NAC in the presence of an acetyl acceptor (e.g., CAR).
114. A method of making a dry blended preparation comprising N-acetylcysteine (NAC) and carnitine (CAR), the method comprising:
   forming a dry blended preparation comprising NAC and CAR, under conditions such that deacetylation of NAC in the dry blended preparation is decreased compared to the deacetylation in a reference mixture,
   wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water,
   thereby making a dry blended preparation comprising NAC and CAR.
115. The method of any of embodiments 112-114, further comprising:
   determining the level of NAC, e.g., at a first time point and at a second time point;
   optionally, comparing the level of NAC to a reference value, e.g., a NAC value between 90-110% of the original level of NAC in the dry blended preparation.
116. The method of any of embodiments 112-115, wherein the conditions comprise one or more (e.g., all) of:
   i) the dry blended preparation comprises an adsorbent and the deacetylation of NAC in the dry blended preparation is decreased compared to the deacetylation in a reference mixture comprising NAC and CAR in the absence of the adsorbent;
   ii) maintaining a water (e.g., free, non-adsorbed water (e.g., able to react or facilitate reaction of NAC)) concentration in the dry blended preparation of less than 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w);
   iii) CAR is provided in a form (e.g., crystal polymorph or alternate salt form) with educed hygroscopicity relative to the free-base form of CAR, e.g., provided in the form of levocarnitine, carnitine HC1, or carnitine tartrate; or
   iv) the dry blended preparation comprises one or more other components (e.g., amino acid entities) in a form (e.g., polymorph) with reduced hygroscopicity, e.g., relative to the free-base form of the amino acid entity.
117. A method of evaluating the stability of N-acetylcysteine (NAC) in a dry blended preparation, the method comprising:
   providing a dry blended preparation comprising NAC; and
   determining the level of NAC, e.g., at a first time point and at a second time point; and
   optionally, comparing the level of NAC to a reference value, e.g., a NAC value between 90-110% of the original level of NAC in the mixture,
   wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water,
   thereby evaluating the stability of NAC in a dry blended preparation.
118. The method of embodiment 117, wherein the dry blended preparation comprises carnitine (CAR).
119. The method of any of embodiments 112-118, wherein the dry blended preparation comprises an adsorbent.
120. The method of any of embodiments 115 or 117-119, wherein determining the level of NAC comprises a method described herein, e.g., HPLC or mass spectrometry, e.g., high resolution mass spectrometry.

10-121. The method of any of embodiments 112-120, wherein forming, or providing, the dry blended preparation comprises:
contacting the NAC with the adsorbent, thereby forming a first mixture; and
contacting the first mixture with CAR, thereby forming the dry blended preparation.
122. The method of any of embodiments 112-120, wherein forming, or providing, the dry blended preparation comprises:
   contacting the CAR with the adsorbent, thereby forming a first mixture; and contacting the first mixture with NAC, thereby forming the dry blended preparation.
123. The method of any of embodiments 112-120, wherein forming, or providing, the dry blended preparation comprises:
   contacting the NAC and CAR with the adsorbent (e.g., simultaneously), thereby forming the dry blended preparation.
124. The method of embodiment 121, wherein the first mixture lacks CAR.
125. The method of embodiment 122, wherein the first mixture lacks NAC.
126. The method of any of embodiments 121-125, wherein the first mixture and/or the dry blended preparation are a powder, e.g., that comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water.
127. A method of pre-treating N-acetylcysteine (NAC), the method comprising:
   providing a mixture of NAC and an adsorbent,
   thereby pre-treating NAC.
128. A method of pre-treating carnitine (CAR), the method comprising:
   providing a mixture of CAR and an adsorbent,
   thereby pre-treating CAR.
129. The method of embodiment 128, wherein the mixture of CAR and adsorbent lacks NAC.
130. The method of embodiment 127, wherein the mixture of NAC and adsorbent lacks CAR.
   20-131. The method of any of embodiments 127-130, wherein the mixture is a dry blended preparation comprising a plurality of amino acid entities.
132. A method of making a dry blended preparation comprising N-acetylcysteine (NAC) and carnitine (CAR), the method comprising:
   providing pre-treated NAC mixed with adsorbent, and
   forming a dry blended preparation comprising pre-treated NAC and CAR,
   thereby making a dry blended preparation comprising NAC and CAR.
133. A method of making a dry blended preparation comprising N-acetylcysteine (NAC) and carnitine (CAR), the method comprising:
   providing pre-treated CAR mixed with adsorbent, and
   forming a dry blended preparation comprising pre-treated CAR and NAC,
   thereby making a dry blended preparation comprising NAC and CAR.
134. The method of embodiment 132, wherein providing pre-treated NAC comprises the method of any of embodiments 127, 130, or 131.
135. The method of embodiment 132, wherein providing pre-treated NAC comprises acquiring, e.g., purchasing, the pre-treated NAC.
136. The method of embodiment 133, wherein providing pre-treated CAR comprises the method of either of embodiments 128 or 129.
137. The method of embodiment 133, wherein providing pre-treated CAR comprises acquiring, e.g., purchasing, the pre-treated CAR.
138. A dry blended preparation comprising:
   N-acetylcysteine (NAC), carnitine (CAR) and an adsorbent (e.g., SiO₂),
   wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, and
   wherein the adsorbent (e.g., SiO₂) is present at a weight percentage (w/w) of at least 0.05, 0.075, 0.1, 0.125, 0.15, 0.175, 0.2, 0.225, 0.25, 0.275, 0.3, 0.325, 0.35, 0.4, 0.425, 0.45, 0.475, 0.5, 0.6, 0.7, 0.8, 0.9, 1, 1.5, 2, 2.5, 3, 3.5, 4, 4.5, or 5%, e.g., at least 0.3%, (and optionally at less than 10, 9, 8, 7, 6, 5, 4, 3, 2, 1.5, 1, 0.9, 0.8, 0.7, 0.6, 0.575, 0.55, 0.525, 0.5, 0.475, 0.45, 0.425, 0.4, 0.375, 0.35, or 0.325%).
139. A dry blended preparation comprising:
   N-acetylcysteine (NAC) and an adsorbent (e.g., SiO₂),
   wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, and
   wherein the adsorbent (e.g., SiO₂) is present at a weight percentage (w/w) of at least 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, 10, 12, 14, 16, 18, 20, 22, 24, 26, 28, 30, 32, 34, 36, 38, or 40% (and optionally at less than 40, 35, 30, 25, 20, 19, 18, 17, 16, 15, 14, 13, 12, 11, 10, 9, 8, 7, or 6.5%).
140. A dry blended preparation comprising: N-acetylcysteine (NAC) and carnitine (CAR), wherein the adsorbent adsorbs to the NAC, e.g., to decrease deacetylation of NAC, e.g., by CAR.
30-141. A dry blended preparation comprising: N-acetylcysteine (NAC) and carnitine (CAR), wherein the adsorbent adsorbs to the CAR, e.g., to decrease deacetylation of NAC, e.g., by CAR.
142. The method of any of embodiments 116 or 119-137 embodiment, wherein pretreating CAR, pretreating NAC, contacting the CAR with the adsorbent, contacting the NAC with the adsorbent, forming the dry blended preparation, or forming the first mixture adsorbs adsorbent to one or both of the CAR or NAC (e.g., as demonstrated by visual microscopy, e.g., by the methods of Example 5).
143. The method or dry blended preparation of any of embodiments 140-142, wherein adsorption comprises surrounding particles of CAR, NAC, or both with an adsorbed layer (e.g., a partial or complete layer) of adsorbent at least 1, 2, 3, 4, 5, 6, 7, 8, 9, 10, 20, 30, 40, or 50 μm thick (and optionally no thicker than 80, 70, 60, 50, 40, 30, 20, or 10 μm).
144. The method or dry blended preparation of any of embodiments 116, 119-137, or 140-143, wherein pretreating CAR, pretreating NAC, contacting the CAR with the adsorbent, contacting the NAC with the adsorbent, forming the dry blended preparation, or forming the first mixture adsorbs adsorbent to the NAC and/or CAR and decreases the concentration of free, non-adsorbed water around NAC and/or CAR relative to the concentration of free, non-adsorbed water around NAC or CAR in the absence of adsorbent.
145. The method or dry blended preparation of embodiment 144, wherein the concentration of free, non-adsorbed water decreases by at least 10, 20, 30, 40, 50, 60, 70, 80, 90, or 100% relative to NAC or CAR in the absence of adsorbent.
146. The method or dry blended preparation of any preceding embodiment, wherein the dry blended preparation, first mixture, pretreated NAC, or pretreated CAR comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water.
147. The method or dry blended preparation of any of embodiments 116 or 119-146, wherein the adsorbent is chosen from: SiO₂, magnesium silicate, calcium silicate, talc, calcium carbonate, magnesium carbonate, MgO, calcium sulfate, CaCl₂, aluminum metal silicate, anhydrous Si acid, magnesium aluminum silicate, microcrystalline cellulose, sodium carboxymethylcellulose, calcium carboxymethylcellulose, or any other appropriate adsorbent.
148. The method or dry blended preparation of any of embodiments 116 or 119-146, wherein the adsorbent is SiO₂.
149. The method or dry blended preparation of either of embodiments 147 or 148, wherein the SiO₂ is colloidal SiO₂.
150. The method or dry blended preparation of any of embodiments 147-149, wherein the SiO₂ is mesoporous.
40-151. The method or dry blended preparation of any of embodiments 147-150, wherein the SiO₂ has an average primary particle size of 2-50, 2-40, 2-30, 2-20, 2-10, 5-50, 5-40, 5-30, 5-20, 5-10, 10-50, 10-40, 10-30, 10-20, 20-50, 20-40, 20-30, 30-50, 30-40, or 40-50 nm.
152. The method or dry blended preparation of any of embodiments 147-151, wherein the SiO₂ has an average surface area of 50-1000, 50-900, 50-800, 50-700, 50-600, 50-500, 50-400, 50-300, 50-200, 50-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-800, 600-700, 700-1000, 700-900, 700-800, 800-1000, 800-900, or 900-1000 m²/g.
153. The method or dry blended preparation of any of embodiments 147-152, wherein the SiO₂ has an average surface area of about 200-300 m²/g.
154. The method or dry blended preparation of any of embodiments 147-153, wherein the SiO₂ has a bulk density of less than 0.1 g/mL.
155. The method or dry blended preparation of any of embodiments 147-154, wherein the SiO₂ is an Aerosil 300 SiO₂, or substantially equivalent thereto.
156. The method or dry blended preparation of any of embodiments 116 or 119-155, wherein the adsorbent, e.g., SiO₂, is present in the first mixture, mixture of NAC and adsorbent (e.g., SiO₂), or mixture of CAR and adsorbent (e.g., SiO₂), at a weight percentage (w/w) of at least 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, 10, 12, 14, 16, 18, 20, 22, 24, 26, 28, 30, 32, 34, 36, 38, or 40% (and optionally at less than 40, 35, 30, 25, 20, 19, 18, 17, 16, 15, 14, 13, 12, 11, 10, 9, 8, 7, or 6.5%).
157. The method or dry blended preparation of any of embodiments 116 or 119-156, wherein the adsorbent, e.g., SiO₂, is present in the first mixture, mixture of NAC and adsorbent (e.g., SiO₂), or mixture of CAR and adsorbent (e.g., SiO₂), at a weight percentage (w/w) of less than or equal to 40, 35, 30, 25, 20, 19, 18, 17, 16, 15, 14, 13, 12, 11, 10, 9, 8, 7, or 6.5% (and optionally at least 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, 10, 12, 14, 16, 18, 20, 22, 24, 26, 28, 30, 32, 34, 36, 38, or 40%).
158. The method or dry blended preparation of any of embodiments 116 or 119-157, wherein the adsorbent (e.g., SiO₂) is present in the dry blended preparation at a weight percentage (w/w) of at least 0.05, 0.075, 0.1, 0.125, 0.15, 0.175, 0.2, 0.225, 0.25, 0.275, 0.3, 0.325, 0.35, 0.4, 0.425, 0.45, 0.475, 0.5, 0.6, 0.7, 0.8, 0.9, 1, 1.5, 2, 2.5, 3, 3.5, 4, 4.5, or 5%, e.g., at least 0.3%, (and optionally at less than 10, 9, 8, 7, 6, 5, 4, 3, 2, 1.5, 1, 0.9, 0.8, 0.7, 0.6, 0.575, 0.55, 0.525, 0.5, 0.475, 0.45, 0.425, 0.4, 0.375, 0.35, or 0.325%).
159. The method or dry blended preparation of any of embodiments 116 or 119-158, wherein the adsorbent (e.g., SiO₂) is present in the dry blended preparation or mixture of adsorbent (e.g., SiO₂), NAC, and CAR at a weight percentage (w/w) of less than or equal to 10, 9, 8, 7, 6, 5, 4, 3, 2, 1.5, 1, 0.9, 0.8, 0.7, 0.6, 0.575, 0.55, 0.525, 0.5, 0.475, 0.45, 0.425, 0.4, 0.375, 0.35, or 0.325% (and optionally of at least 0.05, 0.075, 0.1, 0.125, 0.15, 0.175, 0.2, 0.225, 0.25, 0.275, 0.3, 0.325, 0.35, 0.4, 0.425, 0.45, 0.475, 0.5, 0.6, 0.7, 0.8, 0.9, 1, 1.5, 2, 2.5, 3, 3.5, 4, 4.5, or 5%).
160. The method or dry blended preparation of any of embodiments 116 or 119-159, wherein the NAC of the mixture or dry blended preparation has less deacetylation in the presence of CAR than NAC in a similar mixture or dry blended preparation that does not comprise the adsorbent (e.g., as measured by a stability assay described herein).
50-161. The method or dry blended preparation of any of embodiments 116 or 119-160, wherein the CAR of the mixture or dry blended preparation has less acetylation in the presence of NAC than CAR in a similar mixture or dry blended preparation that does not comprise the adsorbent (e.g., as measured by a stability assay described herein).
162. The method or dry blended preparation of any of embodiments 112-161, wherein at least 90, 91, 92, 93, 94, 95, 96, 97, 98, 98.5, 99, 99.5, or 100% of the NAC of the dry blended preparation remains acetylated after 1, 2, 3, 4, 5, 6, 7, 8, 9, 10, 11, 12, 13, 14, 15, 16, 17, 18, 19, 20, 21, 22, 23, or 24 months at 25° C. and 60% relative humidity (e.g., measured as described in Example 6).
163. The method or dry blended preparation of any of embodiments 112-162, wherein at least 80, 81, 82, 83, 84, 85, 86, 87, 88, 89, 90, 90.5, 91, 91.5, 92, 93, 94, 95, 96, 97, 98, 98.5, 99, 99.5, or 100% of the NAC of the dry blended preparation remains acetylated after 1, 2, 3, 4, 5, or 6 months at 40° C. and 75% relative humidity (e.g., measured as described in Example 6).
164. The method or dry blended preparation of any of embodiments 112-163, wherein the ratio of NAC to CAR in the dry blended preparation is at least 0.1, 0.2, 0.3, 0.4, 0.5, 0.6 0.7, 0.8, 0.9, 1, 1.2, 1.4, 1.6, 1.8, 2, 2.2, 2.4, 2.6, 2.8, 3, 3.2, 3.4, 3.6, 3.8, or 4 after 1, 2, 3, 4, 5, 6, 7, 8, 9, 10, 11, 12, 13, 14, 15, 16, 17, 18, 19, 20, 21, 22, 23, or 24 months at 25° C. and 60% relative humidity (e.g., measured as described in Example 6).
165. The method or dry blended preparation of any of embodiments 112-164, wherein the ratio of NAC to CAR in the dry blended preparation is at least 0.1, 0.2, 0.3, 0.4, 0.5, 0.6 0.7, 0.8, 0.9, 1, 1.2, 1.4, 1.6, 1.8, 2, 2.2, 2.4, 2.6, 2.8, 3, 3.2, 3.4, 3.6, 3.8, or 4 after 1, 2, 3, 4, 5 or 6months at 40° C. and 75% relative humidity (e.g., measured as described in Example 6).
166. The method or dry blended preparation of any of embodiments 112-165, wherein the level of NAC (e.g., w/w) in the dry blended preparation after 1, 2, 3, 4, 5, 6, 7, 8, 9, 10, 11, 12, 13, 14, 15, 16, 17, 18, 19, 20, 21, 22, 23, or 24 months at 25° C. and 60% relative humidity (e.g., measured as described in Example 6) is at least 90, 91, 92, 93, 94, 95, 96, 97, 98, 98.5, 99, 99.5, or 100% of the original level of NAC in the dry blended preparation (and optionally, no more than 110, 109, 108, 107, 106, 105, 104, 103, 102, 101, or 100%).
167. The method or dry blended preparation of any of embodiments 112-166, wherein the level of NAC (e.g., w/w) in the dry blended preparation after 1, 2, 3, 4, 5 or 6 months at 40° C. and 75% relative humidity (e.g., measured as described in Example 6) is at least 90, 91, 92, 93, 94, 95, 96, 97, 98, 98.5, 99, 99.5, or 100% of the original level of NAC in the dry blended preparation (and optionally, no more than 110, 109, 108, 107, 106, 105, 104, 103, 102, 101, or 100%).
168. The method or dry blended preparation of any of embodiments 112-167, wherein the level of a NAC degradation product (e.g., cysteine, cystine, and/or ALCAR), is less than 1% w/w of the dry blended preparation after 1, 2, 3, 4, 5, 6, 7, 8, 9, 10, 11, 12, 13, 14, 15, 16, 17, 18, 19, 20, 21, 22, 23, or 24 months at 25° C. and 60% relative humidity, or after 1, 2, 3, 4, 5, or 6 months at 40° C. and 75% relative humidity, (e.g., measured as described in Example 6).
169. The method or dry blended preparation of any of embodiments 116 or 119-168, wherein acetylation of CAR is decreased compared to acetylation in a reference mixture comprising NAC and CAR in the absence of the adsorbent.
170. The method or amino acid preparation of any of embodiments 116 or 119-169, wherein acetylation of CAR is prevented, e.g., by the presence of the adsorbent.
60-171. The method of any of embodiments 112-137 or 142-170, wherein forming the dry blended preparation comprises blending for a time sufficient time to produce a change in bulk density (e.g., a decrease or increase in bulk density).
172. The method of any of embodiments 112-137 or 142-171, wherein forming the dry blended preparation comprises blending for a time sufficient to produce a mixture having a lower volume than the sum of the volumes of the separate components.
173. The method of any of embodiments 112-137 or 142-172, wherein forming the dry blended preparation comprises blending for a time sufficient to achieve a standard for uniformity, e.g., composition uniformity.
174. The method of any of embodiments 112-137 or 142-173, wherein contacting or forming comprises shaking or inverting the dry blended preparation or first mixture (e.g., in a container, e.g., jar or blender) to coat the NAC or CAR with SiO₂.
175. The method of embodiment 174, wherein shaking or inverting the dry blended preparation or first mixture has a duration of at least 10, 20, 30, 40, 50, or 60 seconds, or 1, 2, 3, 4, 5, 6, 7, 8, 9, 10, 15, 20, 25, 30, 35, 40, 45, 50, 55, or 60 minutes (and optionally, no more than 120, 100, 80, 60, 40, 30, or 15 minutes).
176. The method of embodiment 174, wherein shaking or inverting the dry blended preparation or first mixture continues until the dry blended preparation or first mixture appears uniform, e.g., by visual inspection.
177. The method of any of embodiments 112-137 or 142-176, wherein contacting or forming comprises subjecting the dry blended preparation or first mixture to a blending condition, e.g., that uses convection diffusion, or shear blending mechanism.
178. The method or dry blended preparation of any of embodiments 112-177, wherein the dry blended preparation further comprises one or more amino acid entities chosen from one, two, or all of a leucine amino acid entity, an arginine amino acid entity, or a glutamine amino acid entity.
179. The method or dry blended preparation of any of embodiments 112-178, wherein the dry blended preparation is a pharmaceutical grade dry blended preparation (PGDBP).
180. The method or dry blended preparation of any of embodiments 112-179, wherein the dry blended preparation has been subjected to a downstream processing step, e.g., fill finish, packaging, or labeling.
70-181. The method or dry blended preparation of any of embodiments 112-179, wherein the dry blended preparation is comprised within a finished product, e.g., for use or sale to an end user.
182. The method or dry blended preparation of any of embodiments 112-181, wherein the dry blended preparation further comprises:
   a) a leucine amino acid entity,
   b) an arginine amino acid entity, and/or
   c) a glutamine amino acid entity.
183. The method or dry blended preparation of any of embodiments 112-71, wherein the dry blended preparation further comprises:
   a) a leucine amino acid entity,
   b) an arginine amino acid entity,
   c) a glutamine amino acid entity,
   d) a citrulline amino acid entity,
   e) a serine amino acid entity,
   f) a valine amino acid entity,
   g) a histidine amino acid entity, and
   h) a lysine amino acid entity.
184. The method or dry blended preparation of any of embodiments 116-182, wherein the dry blended preparation further comprises:
   a) a leucine amino acid entity,
   b) an arginine amino acid entity,
   c) a glutamine amino acid entity,
   d) a citrulline amino acid entity,
   e) a serine amino acid entity,
   f) a valine amino acid entity,
   g) a histidine amino acid entity, and
   h) a lysine amino acid entity, and
wherein the adsorbent is SiO₂, e.g., mesoporous and/or colloidal SiO₂, e.g., Aerosil 300 SiO₂ or a substantially equivalent SiO₂.
185. The method or dry blended preparation of any of embodiments 112-182, wherein the dry blended preparation further comprises:
   a) a leucine amino acid entity,
   b) an arginine amino acid entity,
   c) a glutamine amino acid entity,
   d) an isoleucine amino acid entity, and
   e) a serine amino acid entity.
186. The method or dry blended preparation of any of embodiments 116-182, wherein the dry blended preparation further comprises:
   a) a leucine amino acid entity,
   b) an arginine amino acid entity,
   c) a glutamine amino acid entity,
   d) an isoleucine amino acid entity, and
   e) a serine amino acid entity, and
wherein the adsorbent is SiO₂, e.g., mesoporous and/or colloidal SiO₂, e.g., Aerosil 300 SiO₂ or a substantially equivalent SiO₂.
187. A dry blended preparation made by the method of any of embodiments 112-137 or 142-186.
188. A pretreated NAC preparation made by the method of any of embodiments 127, 130, or 131.
189. A pretreated CAR preparation made by the method of either of embodiments 128 or 129.
190. A single dose aqueous composition (e.g., aqueous suspension) comprising:
   a preparation comprising one or more amino acids and a wetting agent, wherein optionally preparation is the dry blended preparation of any of embodiments 13, 14, 27-67, and 100-111; and
   a volume of water,
   wherein the single dose aqueous composition comprises a total amino acid dose of at least 8 grams of amino acids per dose.
191. The single dose aqueous composition of embodiment 190, wherein the total amino acid dose is no more than 28 grams of amino acids per dose, e.g., the total amino acid dose is 8-10, 10-15, 15-20, 20-25, or 25-28 grams of amino acids per dose.
192. The single dose aqueous composition of embodiment 190 or 191, wherein, the total amino acid concentration is at least 1.4 g/oz, optionally wherein the volume of water is 6 oz.
193. The single dose aqueous composition of any of embodiments 190-192, wherein, the total amino acid concentration is no more than 5 g/oz, e.g., wherein the total amino acid concentration is 1.4-2, 2-3, 3-4, or 4-5 g/oz.
194. The single dose aqueous composition of embodiment 190 or 191, wherein, the total amino acid concentration is at least 2 g/oz, optionally wherein the volume of water is 4 oz.
195. The single dose aqueous composition of embodiment 190, 191, or 194, wherein, the total amino acid concentration is no more than 7 g/oz, e.g., wherein the total amino acid concentration is 2-3, 3-4, 4-5, 5-6, or 6-7 g/oz.
196. The single dose aqueous composition of embodiment 190 or 191, wherein the total amino acid concentration is at least 5.5 g/oz, optionally wherein the volume of water is 2 oz.
197. The single dose aqueous composition of embodiment 190,191, or 196, wherein the total amino acid concentration is no more than 14 g/oz, e.g., wherein the total amino acid concentration is 5.5-6, 6-8, 8-10, 10-12, or 12-14 g/oz.
198. A single dose aqueous composition (e.g., aqueous suspension) comprising:
   a preparation comprising one or more amino acids and a wetting agent, wherein optionally preparation is the dry blended preparation of any of embodiments 13, 14, 27-67, and 100-111; and
   a volume of water,
   wherein the single dose aqueous composition comprises a total hydrophobic amino acid (e.g., leucine (L)-amino acid entity, isoleucine (I)-amino acid entity, valine (V)-amino acid entity, phenylalanine (F)-amino acid entity, and tryptophan (W)-amino acid entity) dose of at least 1 gram of total hydrophobic amino acids per dose.
199. The single dose aqueous composition of embodiment 198, wherein the total hydrophobic amino acid dose is no more than 15 grams of amino acids per dose, e.g., wherein the total hydrophobic amino acid dose is 1-3, 3-5, 5-7, 7-9, 9-11, 11-13, or 13-15 grams of amino acids per dose.
200. The single dose aqueous composition of embodiment 198 or 199, wherein the total hydrophobic amino acid concentration is at least 0.2 g/oz, optionally wherein the volume of water is 6 oz.
201. The single dose aqueous composition of any of embodiments 198-200, wherein the total amino acid concentration is no more than 2.5 g/oz, e.g., wherein the total amino acid concentration is 0.2-1, 1-1.5, 1.5-2, or 2-2.5 g/oz.
202. The single dose aqueous composition of embodiment 198 or 199, wherein the total hydrophobic amino acid concentration is at least 0.3 g/oz, optionally wherein the volume of water is 4 oz.
203. The single dose aqueous composition of embodiment 198, 199, or 202, wherein the total hydrophobic amino acid concentration is no more than 4 g/oz, e.g., wherein the total hydrophobic amino acid concentration is 0.3-1, 1-2, 2-3, or 3-4 g/oz.
204. The single dose aqueous composition of embodiment 198 or 199, wherein the total hydrophobic amino acid concentration is at least 0.6 g/oz, optionally wherein the volume of water is 2 oz.
205. The single dose aqueous composition of embodiment 198, 199, or 204, wherein the total hydrophobic amino acid concentration is no more than 7.5 g/oz, e.g., wherein the total hydrophobic amino acid concentration is 2-3, 3-4, 4-5, 5-6, 6-7, or 7-7.5 g/oz.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F show the effects of a wetting agent on dispersion of amino acid entities in an aqueous suspension comprising exemplary dry blended preparation 1 (comprising an Active Moiety). FIG. 1A shows an aqueous suspension comprising a single dose of exemplary dry blended preparation 1 with no wetting agent immediately after shaking (first panel), 5 minutes of holding after shaking (second panel), after resuspension (third panel), or 5 minutes of holding following resuspension (fourth panel). FIG. 1B shows an aqueous suspension comprising a single dose of exemplary dry blended preparation 1 with 60 mg of a wetting agent, lecithin (Alcolec 40P), immediately after shaking (first panel), one hour of holding after shaking (second panel), and 5 minutes of holding after resuspension (performed one hour post-initial shaking) (third panel). FIG. 1C shows an aqueous suspension comprising a single dose of exemplary dry blended preparation 1 with 100 mg of a wetting agent, poloxamer 331, immediately after shaking (first panel), 5 minutes of holding after shaking (second panel), after resuspension (third panel), or 5 minutes of holding following resuspension (fourth panel). FIG. 1D shows a titration assay, in which increasing concentrations from 0 mg to 60 mg of a wetting agent lecithin (Lipoid S20) was included in single dose aqueous suspensions of exemplary dry blended preparation 1. Dispersion of amino acid entities and foaming were evaluated visually immediately following shaking (top left image), 5 minutes of holding post-shaking (bottom left), following resuspension (top right image), and 5 minutes of holding post-resuspension (bottom right image). FIG. 1E shows effects of the inclusion of 60 mg of a wetting agent, lecithin (Alcolec 40P), in exemplary dry blended preparations that are dispersed in different volumes (0.33, 0.5, 1 or 2 ounces) of water. Dispersion of amino acid entities and foaming were evaluated after initial shaking (top panel), 6 minutes of holding after shaking (middle panel), and 5 minutes of holding following resuspension (bottom panel). FIG. 1F shows a visual dose delivery test (amount of material that easily pours out of the preparation container) for a control aqueous suspension comprising exemplary dry blended preparation 1 with no wetting agent (left two tubes) and an aqueous suspension comprising exemplary dry blended preparation 1 with 60 mg of a wetting agent, lecithin (Alcolec 40P) (right tube).

FIGS. 2A-2C show the effects of wetting agents on dispersion of amino acid entities in an aqueous suspension comprising exemplary dry blended preparation 2 (comprising an Active Moiety). FIG. 2A shows the dispersion of amino acid entities in control aqueous suspensions of various volumes (1 dose of amino acid entities added to 4 oz, 2 oz, 1 oz or 0.5 oz of water) with no wetting agent, immediately following shaking (top panel) or 6 minutes of holding after shaking and resuspension (bottom panel). FIG. 2B shows the dispersion of amino acid entities in an aqueous suspension with 60 mg of lecithin (Alcolec 40P) wetting agent at various suspension volumes (4, 2, 1, or 0.5 ounces) per dose, immediately following shaking (top panel) or 5 minutes of holding after shaking and resuspension (bottom panel). FIG. 2C shows a titration assay with increasing concentrations (from 20 mg to 100 mg) of a wetting agent, lecithin (Alcolec 40P), in a single dose of exemplary dry blended preparation 2 used to constitute aqueous suspensions post-initial shaking (top left), 5 minutes of holding post-shaking (bottom left), post-resuspension (top right), and 5 minutes of holding after resuspension (bottom right).

FIGS. 3A-3E show the effects of wetting agents on dispersion of amino acid entities in an aqueous suspension comprising exemplary dry blended preparation 3 (comprising an Active Moiety). FIG. 3A shows effects of wetting agents, lecithin (Alcolec 40P) or poloxamer 331, on dispersion of amino acid entities immediately following constitution (top left), 5 minutes of holding after constitution (top right), 5 minutes of holding after resuspension (that was done 5 minutes of holding after constitution) (bottom left), and 1 hour of holding after constitution (bottom right). FIG. 3B shows the effects of a wetting agent, lecithin (Alcolec 40P), on different aqueous suspension volumes (0.5-2 ounces) per dose comprising exemplary dry blended preparation 3, immediately after shaking (top panel) and 5 minutes of holding after resuspension (that was performed 5 minutes of holding after shaking) (bottom panel). FIG. 3C shows a titration assay with lecithin (Alcolec 40P) in which increasing concentrations of wetting agent from 40 mg to 100 mg were included in a single dose of the dry blended preparation constituted to make aqueous suspensions, post-initial shaking (top left), post-resuspension (top right), 5 minutes of holding post-shaking (bottom left), and 5 minutes of holding after resuspension (bottom right). FIG. 3D shows a titration assay with poloxamer 331 in which increasing concentrations of wetting agent from 50 mg to 200 mg were included in water used to constitute a single dose of dry blended preparation 3 in aqueous suspensions, post-initial shaking (top left), post-resuspension (top right), 5 minutes of holding post-shaking (bottom left), and 5 minutes of holding after resuspension (bottom right). FIG. 3E shows a titration assay with lecithin (Lipoid 20S) (also known as Lipoid S20) in which increasing concentrations of wetting agent from 60 mg to 150 mg were included in a single dose of dry blended preparation 3 used to constitute aqueous suspensions 3, post-initial shaking (top left), post-resuspension (top right), 5 minutes of holding post-shaking (bottom left), and 5 minutes of holding after resuspension (bottom right).

FIGS. 4A-4B show the effects of SiO₂ coating on NAC particles. FIG. 4A shows a composition comprising NAC and SiO₂ at multiple time points (pre-mixing, 30 seconds, 5 minutes and 10 minutes) during the process of mixing the two components. After shaking for 10 minutes, a fully blended mixture of NAC and SiO₂ was obtained. FIG. 4B shows microscopy images of NAC particles coated with SiO₂ after mixing.

### DETAILED DESCRIPTION

The present disclosure is directed, in part, to methods of improving the dispersion of an amino acid, e.g., a hydrophobic amino acid, in an aqueous suspension, comprising providing a dry blended preparation comprising the amino acid (e.g., hydrophobic amino acid), and a wetting agent, and combining the dry blended preparation, wetting agent, and water to form an aqueous suspension that achieves a standard for suspension uniformity. The present disclosure is further directed, in part, to methods of: manufacturing, making, or formulating an aqueous suspension; reducing foaming of an aqueous suspension comprising a hydrophobic amino acid; and increasing the solubilization rate of an amino acid (e.g., hydrophobic amino acid) in an aqueous suspension, comprising providing a dry blended preparation comprising the amino acid (e.g., hydrophobic amino acid), and a wetting agent, and combining the dry blended preparation, wetting agent, and water to form an aqueous suspension that achieves a standard for suspension uniformity. The present disclosure is further directed, in part, to methods of making a dry blended preparation with improved hydrophobic amino acid dispersion comprising providing a dry blended preparation comprising at least one hydrophobic amino acid entity, and a wetting agent. The present disclosure is further directed, in part, to aqueous suspensions and dry blended preparations (e.g., PGDBPs) made, modified, or evaluated by the methods described herein. The following detailed description characterizes these aspects of the invention.

While not wishing to be bound by theory, several amino acids are capable of reacting with one another under typical preparation and storage conditions, including in dry mixtures. This instability decreases the shelf life and value of amino acid mixtures. This disclosure provides, among other things, techniques and compositions to improve the stability and decrease reactivity of amino acids in mixtures.

The present disclosure is further directed, in part, to a method of stabilizing NAC in the presence of an acetyl acceptor, e.g., CAR, in a dry blended preparation, the method comprising forming the dry blended preparation under conditions that decrease acetylation of NAC. Such conditions include but are not limited to: the presence of an adsorbent; maintaining a water (e.g., free non-adsorbed water (e.g., able to react or facilitate reaction of NAC)) concentration in the dry blended preparation of less than 5% or less than 2% (w/w); providing the acetyl acceptor (e.g., CAR) in a form (e.g., crystal polymorph or alternate salt form) with reduced hygroscopicity relative to a free-base form of the acetyl acceptor (e.g., CAR); and/or providing another component (e.g., amino acid entity) in a form (e.g., polymorph) with reduced hygroscopicity, e.g., relative to the free-base form of the amino acid entity. Without wishing to be bound by theory, it is thought that the presence of water facilitates the deacetylation of NAC, e.g., by the acetyl acceptor (e.g., CAR). Decreasing the interaction of NAC with water, e.g., and thus the acetyl acceptor (e.g., CAR), in the dry blended preparation may improve the stability of NAC and the utility of the dry blended preparation in downstream applications, e.g., as a pharmaceutical grade dry blended preparation (PGDBP). The disclosure further provides dry blended preparations produced using the methods described herein. The disclosure further provides methods of pre-treating NAC and/or acetyl acceptor (e.g., CAR) by contacting the NAC or acetyl acceptor (e.g., CAR) with an adsorbent, and the pre-treated NAC and/or acetyl acceptor (e.g., CAR) produced by the same. Without wishing to be bound by theory, it is thought that NAC and/or acetyl acceptor (e.g., CAR) that has been pre-treated with an adsorbent may have increased stability and be useful as a more stable component in a variety of downstream applications, e.g., in the production of dry blended preparations comprising a plurality of amino acid entities.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, the term “adsorbent” refers to any compound: i) having a hygroscopicity and surface area sufficient to adsorb water in a dry blended preparation described herein; ii) that substantially does not react with an amino acid entity described herein (e.g., that does not react with an amino acid entity, or reacts at a level that is pharmaceutically acceptable or suitable for use in food); and iii) that is pharmaceutically acceptable for oral consumption by a mammalian subject, e.g., a human subject. In some embodiments, an adsorbent is generally regarded as safe (GRAS) for oral consumption. In some embodiments, an adsorbent is inorganic, e.g., does not comprise C—H or C—N covalent bonds, e.g., silicates, oxides, carbonates, aluminas, or talc). In some embodiments, an adsorbent is organic; an organic adsorbent is an adsorbent comprising one or more polymers comprising a plurality of C—H bonds (e.g., polycarbophil or cellulose). Adsorbents may include desiccants or hydroscopic compounds. Adsorbents include but are not limited to SiO₂, magnesium trisilicate, magnesium silicate, calcium silicate, talc, magnesium carbonate, magnesium oxide, calcium polycarbophil, calcium sulfate, calcium chloride, aluminum silicate, aluminum oxide, magnesium aluminum silicate, sodium aluminosilicate, cellulose, e.g., microcrystalline cellulose, sodium carboxymethylcellulose, and calcium carboxymethylcellulose.

As used herein, the term “amino acid entity” refers to a levo (L)-amino acid in free form or salt form (or both), the L-amino acid residue in a peptide smaller than 20 amino acid residues (e.g., oligopeptide, e.g., a dipeptide or a tripeptide), a derivative of the amino acid, a precursor of the amino acid, or a metabolite of the amino acid (see, e.g., Table 1). An amino acid entity includes a derivative of the amino acid, a precursor of the amino acid, a metabolite of the amino acid, or a salt form of the amino acid that is capable of effecting biological functionality of the free L-amino acid. An amino acid entity does not include a naturally occurring polypeptide or protein of greater than 20 amino acid residues, either in whole or modified form, e.g., hydrolyzed form.

Salts of amino acids include any ingestible salt. For pharmaceutical compositions, the salt form of an amino acid present in the composition (e.g., the Active Moiety) should be a pharmaceutically acceptable salt. In a specific example, the salt form is the hydrochloride (HCl) salt form of the amino acid.

In some embodiments, the derivative of an amino acid entity comprises an amino acid ester (e.g., an alkyl ester, e.g., an ethyl ester or a methyl ester of an amino acid entity) or a keto-acid.

TABLE 1
Amino acid entities include amino acids, precursors, metabolites,
and derivatives of the compositions described herein.

| | Exemplary | | | |
|---|---|---|---|---|
| | Amino Acid | Precursors | Metabolites | Derivatives |
| Leucine | L-Leucine | Oxo-leucine | HMB (beta-hydroxy- | N-Acetyl- |
| | | | beta-methylbutyrate); | Leucine |
| | | | Oxo-leucine; | |
| | | | Isovaleryl-CoA | |
| Arginine | L-Arginine | Argininosuccinate; | Agmatine; | N-Acetyl- |
| | | Aspartate; | Creatine | Arginine |
| | | Glutamate | | |
| Glutamine | L-Glutamine | Glutamate | Glutamate | N-Acetyl- |
| | | | | Glutamine |
| NAC | N- | Serine; | Glutathione; | Cystine; |
| | Acetylcysteine | Acetylserine; | Cystathionine; | Cysteamine |
| | | Cystathionine | Homocysteine; | |
| | | | Methionine | |
| Citrulline | L-Citrulline | N-hydroxyl-arginine | Argininosuccinate, | |
| | | | Nitric oxide | |
| Carnitine | L-Carnitine | 6-N-trimethyllysine; | | Acetyl-L- |
| | | N6-Trimethyl-3- | | Carnitine |
| | | OH-lysine | | (ALCAR); |
| | | | | Proprionyl-L- |
| | | | | Carnitine |
| | | | | (PLCAR); L- |
| | | | | Carnitine L- |
| | | | | Tartrate |
| Serine | L-Serine | Phosphoserine, P- | Glycine, Tryptophan, | |
| | | hydroxypyruvate, L- | Acetylserine, | |
| | | Glycine | Cystathionine, | |
| | | | Phosphatidylserine | |
| Valine | L-Valine | 2-Oxo-valerate | Isobutryl-CoA | N-Acetyl- |
| | | | | Valine |
| Histidine | L-Histidine | Histidinol; | Carnosine; Histamine; | N-Acetyl- |
| | | Histidinal; | Urocanate | Histidine |
| | | Ribose-5-phosphate | | |
| Lysine | L-Lysine | Diaminopimelate; | Trimethyllysine; | N-Acetyl- |
| | | Aspartate | Saccharopine | Lysine |
| Glycine | L-Glycine | Serine, Sarcosine, | Glutathione, Serine, | |
| | | Betaine, | Creatine | |
| | | Dimethyglycine, | | |
| Isoleucine | L-Isoleucine | 2-Oxo-3-methyl- | 2-Oxo-3-methyl- | N-Acetyl- |
| | | valerate; Threonine | valerate; Methylbutyrl- | Isoleucine |
| | | | CoA | |
| Ornithine | L-Ornithine | L-Arginine, Glycine | Citrulline | |
| Aspartate | L-Aspartate | Fumarate | Adenylosuccinate | |
| Threonine | L-Threonine | Homoserine; O- | Oxobutyrate | N-Acetyl- |
| | | PhosphoHomoserine | | Threonine |

“About” and “approximately” shall generally mean an acceptable degree of error for the quantity measured given the nature or precision of the measurements. Exemplary degrees of error are within 15 percent (%), typically, within 10%, and more typically, within 5% of a given value or range of values.

An “amino acid” refers to an organic compound having an amino group (—NH₂), a carboxylic acid group (—C(═O)OH), and a side chain bonded through a central carbon atom, and includes essential and non-essential amino acids and natural, non-proteinogenic, and unnatural amino acids.

As used herein, the term “Active Moiety” means a combination of two or more amino acid entities that, in aggregate, have the ability to have a biological or therapeutic effect as described herein, e.g., an effect on erythrocyte function, turnover, or synthesis; hemoglobin function, turnover, or synthesis; or vascular function. For example, an Active Moiety can rebalance a metabolic dysfunction in a subject suffering from a disease or disorder. An Active Moiety of the invention can contain other biologically active ingredients. In some examples, the Active Moiety comprises a defined combination of four or more amino acid entities, as set out in detail below. In other embodiments, the Active Moiety consists of a defined combination of amino acid entities, as set out in detail below.

The individual amino acid entities are present in the composition, e.g., Active Moiety, in various amounts or ratios, which can be presented as amount by weight (e.g., in grams), ratio by weight of amino acid entities to each other, amount by mole, amount by weight percent of the composition, amount by mole percent of the composition, caloric content, percent caloric contribution to the composition, etc. Generally, this disclosure will provide grams of amino acid entity in a dosage form, weight percent of an amino acid entity relative to the weight of the composition, i.e., the weight of all the amino acid entities and any other biologically active ingredient present in the composition, or in ratios. In some embodiments, the composition, e.g., Active Moiety, is provided as a pharmaceutically acceptable preparation (e.g., a pharmaceutical product).

As used herein, an “aqueous suspension” refers to a mixture comprising water and a plurality of components, e.g., amino acids, e.g., hydrophobic amino acids, wherein at least one component is not completely solubilized in the mixture. In some embodiments, an aqueous suspension comprises at least one component that is substantially, e.g., completely, solubilized in the mixture. In some embodiments, an aqueous suspension comprises one, two, three, four, five, six, or more (e.g., all) of the components of a dry blended preparation (e.g., a PGDBP) and water.

The term “effective amount” as used herein means an amount of an active of the invention in a composition of the invention, particularly a pharmaceutical composition of the invention, which is sufficient to reduce a symptom and/or improve a condition to be treated (e.g., provide a desired clinical response). The effective amount of an active for use in a composition will vary with the particular condition being treated, the severity of the condition, the duration of treatment, the nature of concurrent therapy, the particular active being employed, the particular pharmaceutically-acceptable excipient(s) and/or carrier(s) utilized, and like factors with the knowledge and expertise of the attending physician.

As used herein, a “hydrophobic amino acid entity” refers to an amino acid entity comprising a primarily non-polar side chain. In some embodiments, a hydrophobic amino acid entity comprises one of the canonical amino acids. In some embodiments, a hydrophobic amino acid entity comprises a non-canonical amino acid. In some embodiments, a hydrophobic amino acid entity comprises a branched-chain amino acid entity (BCAA). Hydrophobic amino acid entities include, but are not limited to, a leucine amino acid entity, an isoleucine amino acid entity, a valine amino acid entity, a tryptophan amino acid entity, and a phenylalanine amino acid entity.

A “pharmaceutical composition” described herein comprises at least one “Active Moiety” and a pharmaceutically acceptable carrier or excipient. In some embodiments, the pharmaceutical composition is used as a therapeutic. Other compositions, which need not meet pharmaceutical standards (GMP; pharmaceutical grade components) can be used as a nutraceutical, a medical food, or as a supplement, these are termed “consumer health compositions.”

The term “pharmaceutically acceptable” as used herein, refers to amino acids, materials, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In a specific embodiment, “pharmaceutically acceptable” means a standard used by the pharmaceutical industry or by agencies or entities (e.g., government or trade agencies or entities) regulating the pharmaceutical industry to ensure one or more product quality parameters are within acceptable ranges for a medicine, pharmaceutical composition, treatment, or other therapeutic. A product quality parameter can be any parameter regulated by the pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA).

The term “pharmaceutically acceptable excipient” refers to an ingredient in a pharmaceutical formulation, other than an active, which is physiologically compatible. A pharmaceutically acceptable excipient can include, but is not limited to, a buffer, a sweetener, a dispersion enhancer, a flavoring agent, a bitterness masking agent, a natural coloring, an artificial coloring, a stabilizer, a solvent, or a preservative. In a specific embodiment, a pharmaceutically acceptable excipient includes one or both of citric acid or lecithin.

The term “non-amino acid entity protein component,” as used herein, refers to a peptide (e.g., a polypeptide or an oligopeptide), a fragment thereof, or a degraded peptide. Exemplary non-amino acid entity protein components include, but are not limited to, one or more of whey protein, egg white protein, soy protein, casein, hemp protein, pea protein, brown rice protein, or a fragment or degraded peptide thereof.

The term “non-protein component,” as used herein, refers to any component of a composition other than a protein component. Exemplary non-protein components can include, but are not limited to, a saccharide (e.g., a monosaccharide (e.g., dextrose, glucose, or fructose), a disaccharide, an oligosaccharide, or a polysaccharide); a lipid (e.g., a sulfur-containing lipid (e.g., alpha-lipoic acid), a long chain triglyceride, an omega 3 fatty acid (e.g., EPA, DHA, STA, DPA, or ALA), an omega 6 fatty acid (GLA, DGLA, or LA), a medium chain triglyceride, or a medium chain fatty acid); a vitamin (e.g., vitamin A, vitamin E, vitamin C, vitamin D, vitamin B6, vitamin B12, biotin, folate, or pantothenic acid); a mineral (zinc, selenium, iron, copper, phosphorous, potassium, manganese, chromium, calcium, or magnesium); or a sterol (e.g., cholesterol).

A composition, formulation or product is “therapeutic” if it provides a desired clinical effect. A desired clinical effect can be shown by lessening the progression of a disease and/or alleviating one or more symptoms of the disease.

A “unit dose” or “unit dosage” comprises the drug product or drug products in the form in which they are marketed for use, with a specific mixture of the active and inactive components (excipients), in a particular configuration (e.g., a capsule shell, for example), and apportioned into a particular dose (e.g., in multiple stick packs).

As used herein, the terms “treat,” “treating,” or “treatment” of a hemoglobinopathy (e.g., sickle cell disease) or a thalassemia refers to ameliorating a hemoglobinopathy or a thalassemia (e.g., slowing, arresting, or reducing the development of a hemoglobinopathy or a thalassemia or at least one of the clinical symptoms thereof); alleviating or ameliorating at least one physical parameter including those which may not be discernible by the patient; and/or preventing or delaying the onset or development or progression of a hemoglobinopathy (e.g., sickle cell disease) or a thalassemia.

A “time sufficient” or “sufficient time” as used herein in the context of blending means a time sufficient to achieve blend and composition uniformity without generating impurities or inducing heterogeneity.

A dry blended preparation, e.g., PGDBP, described herein may be formulated as a “pharmaceutical composition.” A pharmaceutical composition as described herein comprises at least one amino acid entity, e.g., an Active Moiety, and a pharmaceutically acceptable carrier or excipient. In some embodiments, the pharmaceutical composition is used as a therapeutic or a medical food. In some embodiments, the pharmaceutical composition is used as a nutraceutical or as a supplement.

The term “pharmaceutical grade” as used herein, refers to amino acids, materials, excipients, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments, pharmaceutical grade means that the amino acids, materials, or excipients meet the specifications of a monograph, e.g., a monograph of the United States Pharmacopeia (USP), the National Formulary (NF), British Pharmacopeia (BP), European Pharmacopeia (EP), or Japanese Pharmacopeia (JP) detailing tests and acceptance criteria. In some embodiments, the meaning of pharmaceutical grade comprises that the amino acids, excipients, or materials are at least 99% pure.

A dry blended preparation, as used herein, means a combination of a plurality of amino acid entities that substantially lacks water. In some embodiments, a dry blended preparation is a powder. In some embodiments, a dry blended preparation comprises less than or equal to 10, 9, 8, 7, 6, 5, 4, 3, 2, or 1% water by weight. In some embodiments, a dry blended preparation comprises at least 4 amino acid entities, e.g., 4, 5, 6, 7, 8, 9, 10, 11, 12, 13, 14, 15, 16, 17, 18, 19, or 20 amino acid entities.

A pharmaceutical grade dry blended preparation (PGDBP), as used herein, is a dry blended preparation that meets a reference standard (e.g., one or more reference standards) and comprises a plurality of pharmaceutical grade amino acid entities. A PGDBP may be formulated as a pharmaceutical composition, e.g., the PGDBP may further comprise one or more excipients and/or oral administration components. In some embodiments, a reference standard met by a PGDBP is composition uniformity.

Composition uniformity, as used herein, is a standard for the homogeneity of a component of a combination, e.g., a dry blended preparation, e.g., a PGDBP, that comprises blend uniformity, portion uniformity, or both. In some embodiments, a combination meets a standard for composition uniformity, e.g., blend uniformity, if the amount of a component (e.g., a pharmaceutical grade amino acid entity, excipient, or oral administration component) at a sampling point in the combination differs from a reference value by less than a predetermined amount. In some embodiments, the reference value is the amount of the component at a second sampling point in the combination. In some embodiments, the reference value is the amount of the component (e.g., a pharmaceutical grade amino acid entity, excipient, or oral administration component) present in the combination (e.g., a dry blended preparation, e.g., a PGDBP).

In some embodiments, wherein a combination (e.g., a dry blended preparation, e.g., a PGDBP) is divided into portions, the portions of the combination meet a standard for composition uniformity, e.g., portion uniformity, if the amount of a component (e.g., a pharmaceutical grade amino acid entity, excipient, or oral administration component) in a portion differs from a reference value by less than a predetermined amount. In some embodiments, the reference value is the amount of the component in a second portion. In some embodiments, the reference value comprises the amount of the component in N additional portions, wherein N is at least 2, 3, 4, 5, 6, 7, 8, 9, 10, 20, 30, 40, 50, 60, 70, 80, 90, or 100. In some embodiments, the reference value is the amount of the component (e.g., a pharmaceutical grade amino acid entity, excipient, or oral administration component) present in the combination (e.g., a dry blended preparation, e.g., a PGDBP). Amounts may be absolute (e.g., mass or weight) or relative (e.g., percent of total components). In some embodiments, the predetermined amount may be 20, 19, 18, 17, 16, 15, 14, 13, 12, 11, 10, 9, 8, 7, 6, 5, 4, 3, 2, or 1%, e.g., of the reference value. In some embodiments, the predetermined amount is 10% (e.g., the amount of the component differs from the reference value by less than 10%).

Portioning, as used herein, means dividing all or part of the dry blended preparation, e.g., PGDBP, into portions for administration to a patient or subject. The portions created by portioning may be provided in sachets, vials, or other containers, e.g., stick packs. In one embodiment, the portions created by portioning are unit dosage amounts, e.g., one unit dosage or a fraction of a unit dosage (e.g., a stick pack may comprise half a unit dose, such that two stick packs would be used together to provide a single unit dose). In some embodiments, only PGDBPs (e.g., that meet a reference standard) are separated into portions via portioning. In some embodiments, portions generated by portioning also meet a reference standard.

As used herein, “substantially equivalent” refers to the functional and structural similarity of two different compounds, e.g., two wetting agents or two adsorbents. Two compounds are substantially equivalent if they comprise minimally differing structures and/or component parts, and perform similarly in a relevant functional assay. For example, two substantially equivalent wetting agents may both be lecithins which, when included in an aqueous suspension described herein, allow the suspension to achieve a standard for suspension uniformity at a volume X and concentration Y, and structurally differ only in that one lecithin has 20% phosphatidylcholine and the other 21% phosphatidylcholine.

Suspension uniformity, as used herein, refers to a standard for the homogeneity of the distribution of one or more components in an aqueous suspension, e.g., an aqueous suspension comprising a dry blended preparation, e.g., PGDBP, and water. In some embodiments, a standard for suspension uniformity is met when the amount of a component (e.g., a pharmaceutical grade amino acid entity) at a first sampling point in the aqueous suspension differs by no more than a predetermined amount from a reference value. Amounts may be absolute, e.g., grams, or relative, e.g., weight/weight (e.g., X g of the component in Y g of sampling point). Amounts may be arbitrary values, as in the case of comparing absorbance values to absorbance values or in statistical comparisons of curves, e.g., of spectra. In some embodiments, acquiring a value for suspension uniformity comprises assessing a standard for suspension uniformity by acquiring a value for the amount of a component at a first sampling point in the combination and comparing it to reference value. In some embodiments, the reference value is the amount of the component at a second sampling point in the aqueous suspension. In some embodiments, the reference value is the amount of the component (e.g., a pharmaceutical grade amino acid entity (e.g., hydrophobic amino acid entity), excipient, or oral administration component) present in the aqueous suspension or a dry blended preparation, e.g., a PGDBP, constituted into the aqueous suspension.

In some embodiments, the reference value is the amount of the component at a second sampling point in the combination. In some embodiments, the reference value is the amount of the component (e.g., a pharmaceutical grade amino acid entity, excipient, or oral administration component) present in the combination (e.g., a dry blended preparation, e.g., a PGDBP).

As used herein, “wetting agent” refers to any compound that improves the dispersion and/or solubilization of a second compound (e.g., a hydrophobic amino acid entity) in water, wherein the second compound comprises a primarily non-polar moiety. In some embodiments, a wetting agent is an amphipathic compound. In some embodiments, a wetting agent comprises one or more lipid chains. In some embodiments, a wetting agent has an HLB value of 2-3 or 8-9. Wetting agents include, but are not limited to, lecithins and poloxamers.

### Methods of Using Wetting Agents and Compositions Containing the Same

In some embodiments, a dry blended preparation as described herein may be prepared to improve the dispersion of an amino acid in an aqueous suspension, e.g., by including a wetting agent in the dry blended preparation and/or aqueous suspension. The present disclosure is directed, in part, to methods of improving the dispersion of an amino acid, e.g., a hydrophobic amino acid, in an aqueous suspension, comprising providing a dry blended preparation comprising the amino acid (e.g., hydrophobic amino acid), and a wetting agent, and combining the dry blended preparation, wetting agent, and water to form an aqueous suspension that achieves a standard for suspension uniformity. Such a dry blended preparation may also be formed under conditions such that deacetylation of NAC in the dry blended preparation is decreased compared to the deacetylation in a reference mixture, e.g., as described herein. Without wishing to be bound by theory, including a suitable wetting agent in an aqueous suspension comprising a dry blended preparation may improve the dispersion and/or solubilization of one or more amino acids of the dry blended preparation. Inclusion of a suitable wetting agent may more homogenously distribute one or more amino acids (e.g., one or more hydrophobic amino acids) throughout an aqueous suspension, and accordingly improve one or more of mouthfeel, taste, appearance, preparation time (e.g., constitution time) of the aqueous suspension, or dose delivery. Inclusion of a suitable wetting agent may decrease the volume of water required to achieve said improvements or to achieve a standard for suspension uniformity. The present disclosure is further directed, in part, to methods of: manufacturing, making, or formulating an aqueous suspension; reducing foaming of an aqueous suspension comprising a hydrophobic amino acid; and increasing the solubilization rate of an amino acid (e.g., hydrophobic amino acid) in an aqueous suspension, comprising providing a dry blended preparation comprising the amino acid (e.g., hydrophobic amino acid), and a wetting agent, and combining the dry blended preparation, wetting agent, and water to form an aqueous suspension that achieves a standard for suspension uniformity. The present disclosure is further directed, in part, to methods of making a dry blended preparation with improved hydrophobic amino acid dispersion comprising providing a dry blended preparation comprising at least one hydrophobic amino acid entity, and a wetting agent.

In some embodiments, a method of the disclosure comprises providing a dry blended preparation comprising at least one hydrophobic amino acid entity, and a wetting agent. In some embodiments, providing the dry blended preparation comprises producing a dry blended preparation, e.g., by steps or a method described herein. In some embodiments, providing the dry blended preparation comprises acquiring, e.g., purchasing, a dry blended preparation. In some embodiments, providing a dry blended preparation and a wetting agent comprises providing a dry blended preparation comprising the wetting agent. In some embodiments, providing a dry blended preparation and a wetting agent comprises providing a dry blended preparation and providing the wetting agent separate from the dry blended preparation.

In some embodiments, a method of the disclosure comprises combining a dry blended preparation, wetting agent, and water to form an aqueous suspension that achieves a standard for suspension uniformity. In some embodiments, combining comprises combining the dry blended preparation, wetting agent, and water in a single step, e.g., simultaneously.

In some embodiments, combining (e.g., constitution) comprises a plurality of steps comprising combining a plurality of the components of the aqueous suspension in a first step and then adding in one or more remaining component(s) in a subsequent step. In some embodiments combining (e.g., constitution) comprises: combining a dry blended preparation with a wetting agent to form a first mixture, and combining the first mixture with water to form the aqueous suspension. In some embodiments combining (e.g., constitution) comprises: combining a dry blended preparation with water to form a first mixture, and combining the first mixture with a wetting agent to form the aqueous suspension. In some embodiments combining (e.g., constitution) comprises: combining water with a wetting agent to form a first mixture, and combining the first mixture with a dry blended preparation to form the aqueous suspension. For example, a wetting agent that is liquid at room temperature and normal pressure may be combined first with water to form a first mixture, and the first mixture may then be combined with a dry blended preparation to form an aqueous suspension. In some embodiments, a wetting agent that is liquid at room temperature and normal pressure may be combined with a dry blended preparation, e.g., by spraying or wet granulation, to form a first mixture, and the first mixture may then be combined with water to form an aqueous suspension. As a further example, a wetting agent that is a solid, e.g., a powder, at room temperature and normal pressure may be combined first with a dry blended preparation form a first mixture, and the first mixture may then be combined with water to form an aqueous suspension. In some embodiments, a wetting agent that is a solid (e.g., powder) at room temperature and normal pressure may be combined with water to form a first mixture, and the first mixture may then be combined with a dry blended preparation to form an aqueous suspension.

In some embodiments, combining comprises shaking or inverting the dry blended preparation, the wetting agent, and water (e.g., in a container, e.g., jar). In some embodiments, combining comprises employing a blending condition, e.g., a blending condition described herein. In some embodiments, shaking or inverting the dry blended preparation, the wetting agent, and water has a duration of at least 10, 20, 30, 40, 50, or 60 seconds, or 1, 2, 3, 4, 5, 6, 7, 8, 9, 10, 15, 20, 25, 30, 35, 40, 45, 50, 55, or 60 minutes (and optionally, no more than 120, 100, 80, 60, 40, 30, or 15 minutes, or 60, 50, 40, or 30 seconds). In some embodiments, subjecting the dry blended preparation, the wetting agent, and water to a blending condition has a duration of at least 10, 20, 30, 40, 50, or 60 seconds, or 1, 2, 3, 4, 5, 6, 7, 8, 9, 10, 15, 20, 25, 30, 35, 40, 45, 50, 55, or 60 minutes (and optionally, no more than 120, 100, 80, 60, 40, 30, or 15 minutes, or 60, 50, 40, or 30 seconds). In some embodiments, shaking or inverting the dry blended preparation, the wetting agent, and water continues until the aqueous suspension appears uniform, e.g., by visual inspection. In some embodiments, subjecting the dry blended preparation, the wetting agent, and water to a blending condition continues until the aqueous suspension appears uniform, e.g., by visual inspection. In some embodiments, appearing uniform (e.g., by visual inspection) comprises the absence or substantial absence of visible clumps of un-dispersed/un-solubilized material either floating in the suspension or adhered to the side of the container. In some embodiments, shaking or inverting the dry blended preparation, the wetting agent, and water continues until the aqueous suspension achieves a standard for suspension uniformity. In some embodiments, subjecting the dry blended preparation, the wetting agent, and water to a blending condition continues until the aqueous suspension achieves a standard for suspension uniformity.

In some embodiments, combining the dry blended preparation, wetting agent, and water produces an aqueous suspension with a layer of foam on top of the suspension. Without wishing to be bound by theory, it is thought that the layer of foam may result from inefficient dispersion/solubilization of one or more components of the aqueous suspension, e.g., the dry blended preparation (e.g., one or more hydrophobic amino acid entities) and/or the wetting agent. A layer of foam on top of an aqueous suspension may decrease the utility of an aqueous suspension, e.g., by having a deleterious effect on one or more of mouthfeel, taste, appearance, preparation time (e.g., constitution time), suspension uniformity, or dose delivery. A smaller layer of foam and/or a layer of foam which diminishes in size more quickly may result from use of the methods and/or compositions described herein, e.g., including an appropriate wetting agent in an aqueous suspension. In some embodiments, combining the dry blended preparation, wetting agent, and water does not form a layer of foam or forms a layer of foam that is smaller or more rapidly diminishes than is formed when combining a dry blended preparation and water without the wetting agent.

In some embodiments, shaking or inverting the dry blended preparation, wetting agent, and water or subjecting the dry blended preparation, wetting agent, and water to a blending condition is referred to as constitution, e.g., constituting said ingredients into an aqueous suspension. In some embodiments, combining comprises holding the aqueous suspension after constitution. Holding in this context refers to allowing the aqueous suspension to sit without inversion, shaking, or subjecting it to a blending condition, for a duration of time. In some embodiments, holding comprises allowing an aqueous suspension to sit still in a container on a surface for a duration of time. In some embodiments, holding the aqueous suspension reduces the thickness of the layer of foam by at least 10, 20, 30, 40, 50, 60, 70, 80, 90, or 100% (e.g., eliminates the layer of foam), e.g., as measured using visible observation and/or a ruler. In some embodiments, holding the aqueous suspension has a duration of less than 20, 19, 18, 17, 16, 15, 14, 13, 12, 11, 10, 9, 8, 7, 6, 5, 4, or 3 minutes, e.g., less than 10 or 5 minutes, (and optionally at least 10, 20, 30, 40, 50, or 60 seconds, or at least 1, 2, 3, 4, or 5 minutes). In some embodiments, holding the aqueous suspension continues until the aqueous suspension achieves a standard for suspension uniformity.

In some embodiments, combining comprises: shaking or inverting the dry blended preparation, wetting agent, and water or subjecting the dry blended preparation, wetting agent, and water to a blending condition; holding the aqueous suspension for a duration of time; and an additional step of shaking or inverting the dry blended preparation, wetting agent, and water or subjecting the dry blended preparation, wetting agent, and water to a blending condition. In some embodiments, shaking or inverting the dry blended preparation, wetting agent, and water or subjecting the dry blended preparation, wetting agent, and water to a blending condition after a holding step is referred to as reconstitution, e.g., reconstituting said ingredients into an aqueous suspension. In some embodiments, combining comprises a reconstitution step, e.g., to address the inefficient or uneven dispersion/solubilization of one or more components of the aqueous suspension (e.g., dry blended preparation (e.g., one or more hydrophobic amino acid entities) or wetting agent) after constitution and holding. In some embodiments, the shaking or inverting the dry blended preparation, wetting agent, and water or subjecting the dry blended preparation, wetting agent, and water to a blending condition of reconstitution may have the same or different characteristics (e.g., duration, modality, equipment employed) as the analogous steps of constitution. In some embodiments, combining comprises holding the aqueous suspension after reconstitution. In some embodiments, holding the aqueous suspension after reconstitution may have the same or different characteristics (e.g., duration) as a previous holding step.

In some embodiments, combining comprises: constituting the dry blended preparation, wetting agent, and water into an aqueous suspension; and holding the aqueous suspension for a duration of time. In some embodiments, combining comprises: constituting the dry blended preparation, wetting agent, and water into an aqueous suspension; holding the aqueous suspension for a duration of time; and reconstituting the dry blended preparation, wetting agent, and water into the aqueous suspension. In some embodiments, combining comprises: constituting the dry blended preparation, wetting agent, and water into an aqueous suspension; holding the aqueous suspension for a duration of time; reconstituting the dry blended preparation, wetting agent, and water into the aqueous suspension; and holding the aqueous suspension for a duration of time. In some embodiments, combining comprises constituting the dry blended preparation, wetting agent, and water into an aqueous suspension and holding the aqueous suspension for a duration of time, followed by a plurality of cycles of reconstitution and holding, e.g., at least 2, 3, 4, 5, 6, 7, 8, 9, or 10 cycles of reconstitution and holding (and optionally no more than 10, 9, 8, 7, 6, 5, 4, 3, or 2 cycles). Each cycle of reconstitution and holding may be distinct from a previous cycle (e.g., in duration, modality, equipment employed) or similar to a previous cycle.

The present disclosure is further directed to a method of directing provision of an aqueous suspension, the method comprising providing a dry blended preparation comprising at least one hydrophobic amino acid entity, and a wetting agent, and providing instructions for combining the dry blended preparation, the wetting agent, and water to form an aqueous suspension that achieves a standard for suspension uniformity. In some embodiments, instructions provided for combining said ingredients may direct a reader of said instructions to use a combining step described herein.

The present disclosure is further directed to a kit comprising a plurality of amino acid entities, including at least one hydrophobic amino acid entity; a wetting agent; and instructions for combining the plurality of amino acid entities and the wetting agent into an aqueous suspension that achieves a standard for suspension uniformity. In some embodiments, instructions provided for combining said ingredients may direct a reader of said instructions to use a combining step described herein. In some embodiments, the plurality of amino acid entities is a dry blended preparation, e.g., PGDBP. In some embodiments, the wetting agent is provided separated from the plurality of amino acid entities and/or dry blended preparation, e.g., PGDBP. In some embodiments, the plurality of amino acid entities and/or dry blended preparation, e.g., PGDBP, provided comprises the wetting agent.

The present disclosure is further directed to a method of evaluating the dispersion of a dry blended preparation comprising at least one hydrophobic amino acid entity, comprising providing a dry blended preparation comprising at least one hydrophobic amino acid entity, and a wetting agent; combining the dry blended preparation, wetting agent, and water to form an aqueous suspension; and evaluating whether the aqueous suspension achieves a standard for suspension uniformity. In some embodiments, evaluating whether the aqueous suspension achieves a standard for suspension uniformity comprises acquiring a value for the amount of an amino acid entity (e.g., a hydrophobic amino acid entity) at a sampling point (e.g. in weight % of total amino acid entities or weight % of the total mixture), and optionally comparing said value to a reference value.

In some embodiments, a method of improving the dispersion of an amino acid, e.g., a hydrophobic amino acid, in an aqueous suspension, or a method of: manufacturing, making, or formulating an aqueous suspension; reducing foaming of an aqueous suspension comprising a hydrophobic amino acid; or increasing the solubilization rate of an amino acid (e.g., hydrophobic amino acid) in an aqueous suspension, may further comprise evaluating whether the aqueous suspension achieves a standard for suspension uniformity (e.g., as described herein).

### Wetting Agents

The present disclosure is directed, in part, to methods of improving the dispersion of an amino acid, e.g., a hydrophobic amino acid, in an aqueous suspension, by including a wetting agent. A wetting agent refers to any compound that improves the dispersion and/or solubilization of a second compound (e.g., a hydrophobic amino acid entity) in water, wherein the second compound comprises a primarily non-polar moiety. In some embodiments, a wetting agent improves the dispersion and/or solubilization of a hydrophobic amino acid entity in water. In some embodiments, a wetting agent improves the dispersion and/or solubilization of an amino acid entity in water.

Examples of wetting agents are known to those of skill in the art. In some embodiments, a suitable wetting agent will be recognizable by one or more structural or functional characteristics described herein. Examples include, but are not limited to, lecithins, and poloxamers (e.g., and variants thereof).

In some embodiments, a wetting agent has an HLB value within a threshold range. As used herein, “HLB value” refers to the hydrophile-lipophile balance number, used as a measure of the ratio of the hydrophilic and lipophilic character of a compound, e.g., a wetting agent. In some embodiments, a wetting agent has an HLB value of 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. In some embodiments, a wetting agent has an HLB value of 2-3. In some embodiments, a wetting agent has an HLB value of 7-9. In some embodiments, a wetting agent has an HLB value of 8-9. In some embodiments, a wetting agent has an HLB value of 1, 2, 3, 4, 5, 6, 7, 8, 9, or 10. In some embodiments, a wetting agent is a lecithin with an HLB value of 7-9 or 8-9. In some embodiments, a wetting agent is a poloxamer with an HLB value of 1-3 or 2-3.

In some embodiments, a wetting agent modulates (e.g., decreases or increases) the surface tension of an aqueous suspension. Without wishing to be bound by theory, it is thought that an aqueous suspension with a surface tension that falls within a threshold range may have improved dispersion and/or solubilization of an amino acid entity (e.g., a hydrophobic amino acid entity) compared to an aqueous suspension with a surface tension falling outside the threshold range. The identities and levels of components in an aqueous suspension may alter the surface tension of the aqueous suspension and an appropriate wetting agent may modulate the surface tension to be within the threshold range. In some embodiments, an aqueous suspension having a surface tension within a threshold range exhibits efficient wetting, dispersion, and/or solubilization of one or more amino acid entities (e.g., one or more hydrophobic amino acid entities). In some embodiments, a wetting agent, e.g., a surfactant, decreases the surface tension of an aqueous suspension. In some embodiments, a wetting agent increases the surface tension of an aqueous suspension. In some embodiments, the threshold range of surface tension values is 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 70-90, 70-100, 80-90, 80-100, or 90-100 millinewtons per meter. In some embodiments, surface tension of an aqueous suspension is measured by force tensiometer, optical tensiometer, or contact angle.

In some embodiments, a wetting agent is or comprises a lecithin, e.g., one or a mixture of amphipathic glycerophospholipids. Lecithins may be characterized by their phospholipid composition, e.g., in weight %. In some embodiments, a lecithin comprises phosphatidylcholine. In some embodiments, a lecithin comprises phosphatidylinositol. In some embodiments, a lecithin comprises phosphatidylethanolamine. In some embodiments, a lecithin comprises a combination of phosphatidylcholine and phosphatidylinositol. In some embodiments, a lecithin comprises a combination of phosphatidylcholine and phosphatidylethanolamine. In some embodiments, a lecithin comprises a combination of phosphatidylinositol and phosphatidylethanolamine. In some embodiments, a lecithin comprises a combination of phosphatidylcholine, phosphatidylinositol, and phosphatidylethanolamine.

In some embodiments, a lecithin comprises at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% phosphatidylcholine (and optionally less than 50, 45, or 40% phosphatidylcholine). In some embodiments, a lecithin comprises 10-50, 10-40, 10-30, 10-20, 20-50, 20-40, 20-30, 30-50, 30-40, or 40-50% phosphatidylcholine. In some embodiments, a lecithin comprises 20-40% phosphatidylcholine. In some embodiments, a lecithin comprises about 20% phosphatidylcholine.

In some embodiments, a lecithin comprises at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% phosphatidylinositol (and optionally less than 50, 40, 30, 20, 19, 18, 17, 16, or 15% phosphatidylinositol). In some embodiments, a lecithin comprises 10-20, 11-20, 12-20, 13-20, 14-20, 15-20, 16-20, 17-20, 18-20, 19-20, 10-19, 11-19, 12-19, 13-19, 14-19, 15-19, 16-19, 17-19, 18-19, 10-18, 11-18, 12-18, 13-18, 14-18, 15-18, 16-18, 17-18, 10-17, 11-17, 12-17, 13-17, 14-17, 15-17, 16-17, 10-16, 11-16, 12-16, 13-16, 14-16, 15-16, 10-15, 11-15, 12-15, 13-15, 14-15, 10-14, 11-14, 12-14, 13-14, 10-13, 11-13, 12-13, 10-12, 11-12, or 10-11% phosphatidylinositol. In some embodiments, a lecithin comprises 11-15% phosphatidylinositol.

In some embodiments, a lecithin comprises at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% phosphatidylethanolamine (and optionally less than 50, 40, 30, 25, 24, 23, 22, 21, 20, 19, 18, 17, 16, or 15% phosphatidylethanolamine). In some embodiments, a lecithin comprises 10-25, 11-25, 12-25, 13-25, 14-25, 15-25, 16-25, 17-25, 18-25, 19-25, 20-25, 21-25, 22-25, 23-25, 24-25, 10-24, 11-24, 12-24, 13-24, 14-24, 15-24, 16-24, 17-24, 18-24, 19-24, 20-24, 21-24, 22-24, 23-24, 10-23, 11-23, 12-23, 13-23, 14-23, 15-23, 16-23, 17-23, 18-23, 19-23, 20-23, 21-23, 22-23, 10-22, 11-22, 12-22, 13-22, 14-22, 15-22, 16-22, 17-22, 18-22, 19-22, 20-22, 21-22, 10-21, 11-21, 12-21, 13-21, 14-21, 15-21, 16-21, 17-21, 18-21, 19-21, 20-21, 10-20, 11-20, 12-20, 13-20, 14-20, 15-20, 16-20, 17-20, 18-20, 19-20, 10-19, 11-19, 12-19, 13-19, 14-19, 15-19, 16-19, 17-19, 18-19, 10-18, 11-18, 12-18, 13-18, 14-18, 15-18, 16-18, 17-18, 10-17, 11-17, 12-17, 13-17, 14-17, 15-17, 16-17, 10-16, 11-16, 12-16, 13-16, 14-16, 15-16, 10-15, 11-15, 12-15, 13-15, 14-15, 10-14, 11-14, 12-14, 13-14, 10-13, 11-13, 12-13, 10-12, 11-12, or 10-11% phosphatidylethanolamine. In some embodiments, a lecithin comprises 16-22% phosphatidylethanolamine.

In some embodiments, a wetting agent comprises or is Alcolec Lecithin 40P, or a substantially equivalent lecithin. In some embodiments, a wetting agent comprises or is Lipoid 20S Lecithin, or a substantially equivalent lecithin.

In some embodiments, a wetting agent comprises or is a poloxamer. Poloxamers are non-ionic triblock copolymers comprising a central hydrophobic segment of polyoxypropylene flanked by hydrophilic segments of polyoxyethylene, wherein each segment comprises a plurality of monomers. Poloxamers may be referred to as PXYZ, where XY times 100 is the molecular mass of the polyoxypropylene core, and Z times 10 is the % polyoxyethylene. For example, P331 refers to a poloxamer with a poloxypropylene core with a molecular mass of 3300 and 10% polyoxyethylene.

In some embodiments, the wetting agent is or comprises a poloxamer with a polyoxypropylene core molecular mass of at least 2500, 2600, 2700, 2800, 2900, 3000, 3100, 3200, or 3300 g/mol (and optionally no more than 4000, 3900, 3800, 3700, 3600, 3500, 3400, or 3300 g/mol). In some embodiments, the poloxamer has a polyoxypropylene core molecular mass of 2500-4000, 2500-3900, 2500-3800, 2500-3700, 2500-3600, 2500-3500, 2500-3400, 2500-3300, 2500-3200, 2500-3100, 2500-3000, 2500-2900, 2500-2800, 2500-2700, 2500-2600, 2600-4000, 2600-3900, 2600-3800, 2600-3700, 2600-3600, 2600-3500, 2600-3400, 2600-3300, 2600-3200, 2600-3100, 2600-3000, 2600-2900, 2600-2800, 2600-2700, 2700-4000, 2700-3900, 2700-3800, 2700-3700, 2700-3600, 2700-3500, 2700-3400, 2700-3300, 2700-3200, 2700-3100, 2700-3000, 2700-2900, 2700-2800, 2800-4000, 2800-3900, 2800-3800, 2800-3700, 2800-3600, 2800-3500, 2800-3400, 2800-3300, 2800-3200, 2800-3100, 2800-3000, 2800-2900, 2900-4000, 2900-3900, 2900-3800, 2900-3700, 2900-3600, 2900-3500, 2900-3400, 2900-3300, 2900-3200, 2900-3100, 2900-3000, 3000-4000, 3000-3900, 3000-3800, 3000-3700, 3000-3600, 3000-3500, 3000-3400, 3000-3300, 3000-3200, 3000-3100, 3100-4000, 3100-3900, 3100-3800, 3100-3700, 3100-3600, 3100-3500, 3100-3400, 3100-3300, 3100-3200, 3200-4000, 3200-3900, 3200-3800, 3200-3700, 3200-3600, 3200-3500, 3200-3400, 3200-3300, 3300-4000, 3300-3900, 3300-3800, 3300-3700, 3300-3600, 3300-3500, 3300-3400, 3400-4000, 3400-3900, 3400-3800, 3400-3700, 3400-3600, 3400-3500, 3500-4000, 3500-3900, 3500-3800, 3500-3700, 3500-3600, 3600-4000, 3600-3900, 3600-3800, 3600-3700, 3700-4000, 3700-3900, 3700-3800, 3800-4000, 3800-3900, or 3900-4000 g/mol. In some embodiments, the wetting agent is or comprises a poloxamer with a polyoxypropylene core molecular mass of about 3300 g/mol.

In some embodiments, the wetting agent is or comprises a poloxamer that is at least 10, 20, 30, 40, 50, 60, 70, 80, or 90% polyoxyethylene (and optionally, less than 100, 90, 80, 70, 60, 50, 40, 30, or 20%). In some embodiments, the wetting agent is or comprises a poloxamer that is 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100% polyoxyethylene. In some embodiments, the wetting agent is or comprises a poloxamer with about 10% polyoxyethylene.

In some embodiments, the wetting agent is or comprises poloxamer P331 or a substantially equivalent poloxamer.

### Mixtures Comprising Wetting Agents

The present disclosure is directed, in part, to a dry blended preparation comprising at least one hydrophobic amino acid and a wetting agent, to an aqueous suspension comprising said dry blended preparation, and to methods of making and using the same. Without wishing to be bound by theory, it is desirable to provide the plurality of amino acid entities (e.g., an Active Moiety) of a dry blended preparation (e.g., PGDBP) in an aqueous suspension having a low volume, e.g., for ease of administration and use by a subject, e.g., a human subject, e.g., a human patient. Accordingly, the disclosure is directed in part to dry blended preparations and aqueous suspensions that can more effectively provide high concentrations of amino acids at lower volumes of aqueous suspension.

In some embodiments, an aqueous suspension comprises a volume that is less than a threshold volume or within a volume range. In some embodiments, volume is expressed in ounce (fluid ounce (the US customary fluid ounce)). In some embodiments, the volume of an aqueous suspension is less than or equal to 8, 7, 6, 5, 4, 3, 2, or 1 ounce. In some embodiments, the volume of an aqueous suspension is at least about 0.25, 0.33, 0.5, 0.66, 0.75, 1, 1.5, 2, 2.5, 3, 3.5, 4, 4.5, 5, 5.5, or 6 ounces. In some embodiments, the volume of an aqueous suspension is no more than 6, 5.5, 5, 4.5, 4, 3.5, 3, 2.5, 2, 1.5, 1, 0.75, 0.66, 0.5, 0.33, or 0.25 ounces. In some embodiments, the volume of an aqueous suspension is 0.1-6, 0.25-6, 0.33-6, 0.5-6, 0.66-6, 0.75-6, 1-6, 1.5-6, 2-6, 2.5-6, 3-6, 3.5-6, 4-6, 4.5-6, 5-6, 5.5-6, 0.1-5.5, 0.25-5.5, 0.33-5.5, 0.5-5.5, 0.66-5.5, 0.75-5.5, 1-5.5, 1.5-5.5, 2-5.5, 2.5-5.5, 3-5.5, 3.5-5.5, 4-5.5, 4.5-5.5, 0.1-5, 0.25-5, 0.33-5, 0.5-5, 0.66-5, 0.75-5, 1-5, 1.5-5, 2-5, 2.5-5, 3-5, 3.5-5, 4-5, 4.5-5, 0.1-4.5, 0.25-4.5, 0.33-4.5, 0.5-4.5, 0.66-4.5, 0.75-4.5, 1-4.5, 1.5-4.5, 2-4.5, 2.5-4.5, 3-4.5, 3.5-4.5, 4-4.5, 0.1-4, 0.25-4, 0.33-4, 0.5-4, 0.66-4, 0.75-4, 1-4, 1.5-4, 2-4, 2.5-4, 3-4, 3.5-4, 0.1-3.5, 0.25-3.5, 0.33-3.5, 0.5-3.5, 0.66-3.5, 0.75-3.5, 1-3.5, 1.5-3.5, 2-3.5, 2.5-3.5, 3-3.5, 0.1-3, 0.25-3, 0.33-3, 0.5-3, 0.66-3, 0.75-3, 1-3, 1.5-3, 2-3, 2.5-3, 0.1-2.5, 0.25-2.5, 0.33-2.5, 0.5-2.5, 0.66-2.5, 0.75-2.5, 1-2.5, 1.5-2.5, 2-2.5, 0.1-2, 0.25-2, 0.33-2, 0.5-2, 0.66-2, 0.75-2, 1-2, 1.5-2, 0.1-1.5, 0.25-1.5, 0.33-1.5, 0.5-1.5, 0.66-1.5, 0.75-1.5, 1-1.5, 0.1-1, 0.25-1, 0.33-1, 0.5-1, 0.66-1, 0.75-1, 0.1-0.75, 0.25-0.75, 0.33-0.75, 0.5-0.75, 0.66-0.75, 0.1-0.66, 0.25-0.66, 0.33-0.66, 0.5-0.66, 0.1-0.5, 0.25-0.5, 0.33-0.5, 0.1-0.33, 0.25-0.33, or 0.1-0.25 ounces. In some embodiments, the volume of an aqueous suspension is about 2, 1, 0.5, 0.33, or 0.25 ounces.

In some embodiments, an aqueous suspension comprises an overall amino acid concentration of at least a threshold concentration or within a concentration range, and/or a hydrophobic amino acid concentration of at least a threshold concentration or within a concentration range. In some embodiments, concentration is expressed in grams per ounce. As used herein, grams per ounce (g/ounce or g/oz) refers to a unit of concentration corresponding to the metric unit of mass grams per the fluid ounce (the US customary fluid ounce). As used herein, “overall amino acid concentration” refers to a total concentration value for the level of all amino acids, e.g., amino acid entities, present in a composition or mixture, e.g., an aqueous suspension. As used herein, “hydrophobic amino acid concentration” refers to a concentration value for the level of all hydrophobic amino acids, e.g., hydrophobic amino acid entities, present in a composition or mixture, e.g., an aqueous suspension.

In some embodiments, an aqueous suspension has an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce. In some embodiments, an aqueous suspension has an overall amino acid concentration of less than or equal to 15, 10, 9, 8, 7, 6, 5, 4, 3, 2, or 1 g/ounce (and optionally at least 0.7 g/ounce). In some embodiments, an aqueous suspension has an overall amino acid concentration of 0.7-15, 0.7-10, 0.7-9, 0.7-8, 0.7-7, 0.7-6, 0.7-5, 0.7-4, 0.7-3, 0.7-2, 0.7-1, 1-15, 1-14, 1-13, 1-12, 1-11, 1-10, 1-9, 1-8, 1-7, 1-6, 1-5, 1-4, 1-3, 1-2, 2-15, 2-14, 2-13, 2-12, 2-11, 2-10, 2-9, 2-8, 2-7, 2-6, 2-5, 2-4, 2-3, 3-15, 3-14, 3-13, 3-12, 3-11, 3-10, 3-9, 3-8, 3-7, 3-6, 3-5, 3-4, 4-15, 4-14, 4-13, 4-12, 4-11, 4-10, 4-9, 4-8, 4-7, 4-6, 4-5, 5-15, 5-14, 5-13, 5-12, 5-11, 5-10, 5-9, 5-8, 5-7, 5-6, 6-15, 6-14, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11, 9-10, 10-15, 10-14, 10-13, 10-12, 10-11, 11-15, 11-14, 11-13, 11-12, 12-15, 12-14, 12-13, 13-15, 13-14, or 14-15 g/ounce.

In some embodiments, an aqueous suspension has a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce. In some embodiments, an aqueous suspension has a hydrophobic amino acid concentration of less than or equal to 10, 9, 8, 7, 6, 5, 4, 3, 2, 1, or 0.5 g/ounce (and optionally at least 0.05 g/ounce). In some embodiments, an aqueous suspension has a hydrophobic amino acid concentration of 0.05-5, 0.05-4.5, 0.05-4, 0.05-3.5, 0.05-3, 0.05-2.5, 0.05-2, 0.05-1.5, 0.05-1, 0.05-0.5, 0.05-0.1, 0.1-5, 0.1-4.5, 0.1-4, 0.1-3.5, 0.1-3, 0.1-2.5, 0.1-2, 0.1-1.5, 0.1-1, 0.1-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5 g/ounce.

In some embodiments, an aqueous suspension comprises a wetting agent concentration of at least a threshold concentration, no more than a threshold concentration, or within a concentration range. In some embodiments, a dry blended preparation (e.g., PGDBP) comprises a wetting agent concentration of at least a threshold concentration, no more than a threshold concentration, or within a concentration range.

In some embodiments, an aqueous suspension comprises a wetting agent, e.g., lecithin or poloxamer, at a concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, or 3% (w/w) (and optionally less than or equal to 3, 2, 1, or 0.5% (w/w)). In some embodiments, an aqueous suspension comprises a wetting agent, e.g., lecithin or poloxamer, at a concentration of less than or equal to 3, 2, 1, or 0.5% (w/w) (and optionally at least 0.05, 0.1, 0.5, or 1% (w/w)). In some embodiments, an aqueous suspension comprises a wetting agent, e.g., lecithin or poloxamer, at a concentration of 0.05-3, 0.05-2.5, 0.05-2, 0.05-1.5, 0.05-1, 0.05-0.5, 0.05-0.1, 0.1-3, 0.1-2.5, 0.1-2, 0.1-1.5, 0.1-1, 0.1-0.5, 0.5-3, 0.5-2.5, 0.5-2, 0.5-1.5, 0.5-1, 1-3, 1-2.5, 1-2, 1-1.5, 1.5-3, 1.5-2.5, 1.5-2, 2-3, 2-2.5, or 2.5-3% (w/w).

In some embodiments, a dry blended preparation (e.g., PGDBP) comprises a wetting agent, e.g., lecithin or poloxamer, at a concentration of at least 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, or 2.5% (w/w) (and optionally less than or equal to 5, 4, 3, 2, 1.9, 1.8, 1.7, 1.6, 1.5, 1.4, 1.3, 1.2, 1.1, 1, 0.9, 0.8, 0.7, 0.6, or 0.5% (w/w)). In some embodiments, a dry blended preparation (e.g., PGDBP) comprises a wetting agent, e.g., lecithin or poloxamer, at a concentration of less than or equal to 5, 4, 3, 2, 1.9, 1.8, 1.7, 1.6, 1.5, 1.4, 1.3, 1.2, 1.1, 1, 0.9, 0.8, 0.7, 0.6, or 0.5% (w/w) (and optionally at least 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, or 2.5%). In some embodiments, a dry blended preparation (e.g., PGDBP) comprises a wetting agent, e.g., lecithin or poloxamer, at 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5% (w/w).

In some embodiments, the solubilization rate of an amino acid entity, e.g., a hydrophobic amino acid entity, is increased in an aqueous suspension comprising a dry blended preparation and a wetting agent relative to a similar aqueous suspension comprising a dry blended preparation and not comprising the wetting agent. In some embodiments, the solubilization rate of an amino acid entity, e.g., a hydrophobic amino acid entity, is increased by at least 10, 20, 30, 40, 50, 60, 70, 80, 90, 100, 150, 200, 300, 400, 500, or 1000% relative to a similar suspension not comprising the wetting agent. In some embodiments, the solubilization rate of an amino acid entity, e.g., a hydrophobic amino acid entity, is measured using a Type II dissolution apparatus with buffer where a powder (e.g., dry blended preparation and/or wetting agent) may be added. In some embodiments, mixing in such an apparatus is performed at 50-125 rpm with samples taken at various times and analyzed by an appropriate method (HPLC, UPLC, LC-MS) for amino acid content. In some embodiments, such an assay compares dissolution of amino acid entities at the same conditions (e.g., buffer type, volume, mixing speed, and/or temperature) with or without a wetting agent.

### Methods of Using Adsorbents and Compositions Containing the Same

In some embodiments, an aqueous suspension or dry blended preparation as described herein may be prepared to stabilize N-acetylcysteine (NAC) in the presence of an acetyl acceptor (e.g., carnitine (CAR)), e.g., by including an adsorbent in the dry blended preparation. The present disclosure is directed, in part, to methods of stabilizing NAC in the presence of an acetyl acceptor, e.g., CAR, comprising forming a dry blended preparation comprising NAC and an acetyl acceptor, e.g., CAR, under conditions such that deacetylation of NAC in the dry blended preparation is decreased compared to the deacetylation in a reference mixture. In some embodiments, the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water. In some embodiments, stabilizing NAC comprises decreasing the deacetylation of NAC in the presence of acetyl acceptor, e.g., CAR. The present disclosure is further directed, in part, to methods of making an amino acid preparation comprising NAC and acetyl acceptor, e.g., CAR, where a NAC and acetyl acceptor, e.g., CAR, dry blended preparation is formed under conditions such that deacetylation of NAC in the dry blended preparation is decreased compared to the deacetylation in a reference mixture (e.g., a similar dry blended preparation not formed under the deacetylation reducing conditions).

In some embodiments, the acetyl acceptor is carnitine (CAR). As used herein, CAR refers to L-carnitine as a free base or any salt thereof, and any crystal polymorph variants thereof. In some embodiments, CAR comprises or consists of L-carnitine as a free base. In some embodiments, CAR comprises or consists of a salt of L-carnitine. In some embodiments, CAR comprises or consists of carnitine HCl or carnitine tartrate.

In the presence of a suitable acetyl acceptor, e.g., CAR, and water, NAC may undergo deacetylation. Deacetylation may occur in the presence of even the relatively low amounts of water present in a dry blended preparation as described herein. In some embodiments, deacetylation of NAC is undesirable because it alters the level of NAC present in an amino acid preparation. Precise, accurate, and stable (e.g., over time) levels of amino acid entities (e.g., an Active Moiety) are important for amino acid preparations for administration to subjects, e.g., human subjects. Pharmaceutical standards require the levels of ingredients of pharmaceutical compositions to stay within threshold ranges for the expected useful life of the composition and gradual deacetylation of NAC could shorten the expected useful life of an amino acid preparation. Deacetylation of NAC also generates undesirable byproducts, e.g., acetylated CAR and other contaminating degradation products, which may have deleterious effects on the utility of the amino acid preparation.

In some embodiments, conditions that stabilize NAC (e.g., decrease deacetylation of NAC) in the presence of acetyl acceptor, e.g., CAR, comprise including an adsorbent in the dry blended preparation. Without wishing to be bound by theory, an adsorbent may adsorb the low levels of water present in the dry blended preparation, e.g., preventing the water from facilitating the deacetylation of NAC, e.g., by CAR. In some embodiments, the conditions comprise including an adsorbent that reduces the level of water, e.g., free, non-adsorbed water (e.g., able to react or facilitate reaction of NAC) to or maintains the level of water, e.g., free, non-adsorbed water (e.g., able to react or facilitate reaction of NAC) at a level less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w). Additionally or alternately, without wishing to be bound by theory, an adsorbent may adsorb to the surface of NAC or acetyl acceptor particles, e.g., acting as a barrier to (e.g., a partial or complete coating that prevents or decreases) NAC or acetyl acceptor interacting with water. In some embodiments, NAC or the acetyl acceptor, e.g., CAR, is coated or encapsulated with adsorbent. In some embodiments, conditions comprise surrounding particles of NAC, acetyl acceptor (e.g., CAR), or both with an adsorbed layer of adsorbent at least 1, 2, 3, 4, 5, 6, 7, 8, 9, 10, 20, 30, 40, or 50 μm thick (and optionally no thicker than 80, 70, 60, 50, 40, 30, 20, or 10 μm), e.g., 1-25, 5-25, 10-25, 15-25, 20-25, 1-20, 5-20, 10-20, 15-20, 1-15, 5-15, 10-15, 1-10, 5-10, or 1-5 μm thick.

In some embodiments, conditions that stabilize NAC (e.g., decrease deacetylation of NAC) in the presence of acetyl acceptor, e.g., CAR, comprise maintaining a level of water, e.g., free, non-adsorbed water (e.g., able to react or facilitate reaction of NAC), at a level less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w). Without wishing to be bound by theory, it is thought that water may facilitate the deacetylation of NAC, such that reducing the level of water will decrease or prevent the deacetylation of NAC.

In some embodiments, conditions that stabilize NAC (e.g., decrease deacetylation of NAC) in the presence of acetyl acceptor, e.g., CAR, comprise providing the acetyl acceptor, e.g., CAR, in a form (e.g., crystal polymorph or alternate salt form) with reduced hygroscopicity relative to the free-base form of CAR. Without wishing to be bound by theory, an acetyl acceptor that has lower hygroscopicity may have a decreased propensity to react with NAC due to the lower level of water in close proximity to the acetyl acceptor.

In some embodiments, conditions that stabilize NAC (e.g., decrease deacetylation of NAC) in the presence of acetyl acceptor, e.g., CAR, comprise providing another mixture component, e.g., an amino acid entity not comprising NAC or CAR, in a form (e.g., crystal polymorph or alternate salt form) with reduced hygroscopicity relative to the free-base form of the mixture component. Without wishing to be bound by theory, it is thought that water may facilitate the deacetylation of NAC, such that reducing the level of water will decrease or prevent the deacetylation of NAC and using less hygroscopic but similarly active (e.g., bioactive) forms of other mixture components may decrease the level of water in the dry blended preparation.

In some embodiments, a method described herein (employing conditions that stabilize NAC, e.g., decrease deacetylation, in the presence of an acetyl acceptor, e.g., CAR) stabilizes NAC, e.g., decreases deacetylation, relative to a similar reference dry blended preparation not comprising an adsorbent, with a higher level of water (e.g., free, non-adsorbed water (e.g., able to react or facilitate reaction of NAC)), using a more hygroscopic acetyl acceptor (e.g., CAR), and/or using a more hygroscopic other mixture component.

In some embodiments, a method described herein comprises determining the level of NAC in a dry blended preparation, e.g., at a first time point, e.g., and at a second time point. In some embodiments, the method comprises comparing the level of NAC (e.g., at a first time point, e.g., and at a second time point) to a reference value. The reference value may be an acceptable threshold range (e.g., 90-110% of the original level of NAC in the dry blended preparation or 90-110% of the level of NAC at a first time point), or a value or range of values specified by a national or regional pharmacopeia or formulary.

### Adsorbents

The present disclosure is directed, in part, to a composition comprising an adsorbent, e.g., SiO₂. In some embodiments, the composition comprises one, two, three, four, five, six, seven, eight, nine, or ten different adsorbents. In some embodiments, the composition comprises a single adsorbent.

In some embodiments, an adsorbent is chosen from SiO₂, Magnesium trisilicate, Magnesium silicate, Calcium silicate, Talc, Magnesium carbonate, Magnesium Oxide, Calcium Polycarbophil, Calcium sulfate, Calcium chloride, Aluminum silicate, Aluminum Oxide, Magnesium Aluminum Silicate, Sodium aluminosilicate, Cellulose, Microcrystalline cellulose, Sodium carboxymethylcellulose, Calcium carboxymethylcellulose, or any other appropriate adsorbant.

In some embodiments, the adsorbent is SiO₂. In some embodiments, the SiO₂ is colloidal SiO₂. In some embodiments, the SiO₂ is mesoporous. In some embodiments, the SiO₂ has an average primary particle size of 2-50, 2-40, 2-30, 2-20, 2-10, 5-50, 5-40, 5-30, 5-20, 5-10, 10-50, 10-40, 10-30, 10-20, 20-50, 20-40, 20-30, 30-50, 30-40, or 40-50 nm (in diameter). Primary particle size corresponds to the predominant range of sizes of un-agglomerated particles in a mixture of particles (e.g., if at least 50, 60, 70, 80, 90, 95, 99, or 100% of un-agglomerated particles in a mixture of particles have a particle size within a given range, that range is the primary particle size). In some embodiments, the SiO₂ has a maximum particle size of no more than 500, 450, 400, 350, 300, 250, 200, 150, 100, 50, or 10 μm (and optionally, at least 2, 5, 10, 20, 30, 40, or 50 nm).

In some embodiments, the SiO₂ has an average surface area of 50-1000, 50-900, 50-800, 50-700, 50-600, 50-500, 50-400, 50-300, 50-200, 50-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-800, 600-700, 700-1000, 700-900, 700-800, 800-1000, 800-900, or 900-1000 m²/g. In some embodiments, the SiO₂ has an average surface area of 100-400, 100-300, 100-200, 200-400, 200-300, or 300-400 m²/g, e.g., about 100, 200, 300, or 400 m²/g. Without wishing to be bound by theory, higher surface area of an adsorbent correlates with higher hygroscopicity. In some embodiments an adsorbent with a higher surface area may be advantageous to use in a method described herein, e.g., due to its superior ability to stabilize NAC in the presence of acetyl acceptor, e.g., CAR; decrease acetylation of NAC in the presence of acetyl acceptor, e.g., CAR; or pretreat NAC or acetyl acceptor, e.g., CAR, relative to an adsorbent with a lower surface area.

In some embodiments, the SiO₂ has a bulk density that is less than 1, 0.9, 0.8, 0.7, 0.6, 0.5, 0.4, 0.3, 0.2, 0.1, 0.075, 0.05, or 0.025 g/ml, e.g., less than 0.1 g/ml (and optionally at least 0.01 g/ml).

### Mixtures Comprising Adsorbents

In some embodiments, methods described herein comprise forming or providing a dry blended preparation comprising NAC and an acetyl acceptor, e.g., CAR. In some embodiments, forming or providing comprises contacting the NAC with the adsorbent, thereby forming a first mixture, e.g., dry blended preparation; and contacting the first mixture with the acetyl acceptor (e.g., CAR), thereby forming a second mixture (the dry blended preparation). In some embodiments, forming or providing comprises contacting the acetyl acceptor (e.g., CAR) with the adsorbent, thereby forming a first mixture; and contacting the first mixture with the NAC, thereby forming a second mixture (the dry blended preparation). In some embodiments, forming or providing comprises contacting the NAC and CAR with the adsorbent (e.g., simultaneously), thereby forming the mixture. In some embodiments, the first mixture, e.g., comprising NAC and adsorbent, lacks acetyl acceptor (e.g., CAR). In some embodiments, the first mixture, e.g., comprising acetyl acceptor (e.g., CAR) and adsorbent, lacks NAC. In some embodiments, the first mixture and/or second mixture comprise less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water.

The present disclosure is further directed, in part, to a method of pre-treating NAC comprising providing a mixture, e.g., dry blended preparation, of NAC and an adsorbent. The present disclosure is further directed, in part, to a method of pre-treating acetyl acceptor (e.g., CAR) comprising providing a mixture, e.g., dry blended preparation, of acetyl acceptor (e.g., CAR) and an adsorbent. In some embodiments, providing a mixture comprises contacting NAC or acetyl acceptor (e.g., CAR) with the adsorbent. In some embodiments, providing a mixture comprises acquiring, e.g., purchasing, pre-treated NAC or acetyl acceptor (e.g., CAR). Without wishing to be bound by theory, for reasons discussed herein it is thought that treating NAC with adsorbent may stabilize NAC (e.g., decrease deacetylation of NAC) in the presence of an acetyl acceptor (e.g., CAR). It is similarly thought that treating acetyl acceptor, e.g., CAR), with adsorbent may stabilize NAC (e.g., decrease deacetylation of NAC); a decrease in water level or adsorbed adsorbent may function to prevent or decrease interaction of NAC and acetyl acceptor regardless of which participant of the interaction is treated with adsorbent. In some embodiments, both NAC and acetyl acceptor (e.g., CAR) are pre-treated, such a method comprising providing a mixture, e.g., dry blended preparation, of NAC and an adsorbent, providing a mixture, e.g., dry blended preparation, of acetyl acceptor (e.g., CAR) and an adsorbent), and combining the mixture of NAC and adsorbent and the mixture of acetyl acceptor (e.g., CAR) and adsorbent, e.g., to form a dry blended preparation.

The present disclosure is further directed, in part, to methods of making an amino acid preparation comprising NAC and an acetyl acceptor (e.g., CAR), comprising combining:
i) Pre-treated NAC described herein and acetyl acceptor (e.g., CAR),
ii) Pre-treated acetyl acceptor (e.g., CAR) described herein and NAC, or
iii) Pre-treated NAC described herein and Pre-treated acetyl acceptor (e.g., CAR) described herein.

In some embodiments, an adsorbent is present in a mixture (e.g., dry blended preparation) comprising NAC and adsorbent or a mixture (e.g., dry blended preparation) of acetyl acceptor (e.g., CAR) and adsorbent (e.g., a first mixture as described herein, pre-treated NAC, or pre-treated acetyl acceptor (e.g., CAR)) at a weight percentage (w/w) of at least 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, or 10, 12, 14, 16, 18, 20, 22, 24, 26, 28, 30, 32, 34, 36, 38, or 40% (and optionally at less than 40, 35, 30, 25, 20, 19, 18, 17, 16, 15, 14, 13, 12, 11, 10, 9, 8, 7, or 6.5%). In some embodiments, an adsorbent is present in a mixture (e.g., dry blended preparation) of NAC and adsorbent or a mixture (e.g., dry blended preparation) of acetyl acceptor (e.g., CAR) and adsorbent (e.g., a first mixture as described herein, pre-treated NAC, or pre-treated acetyl acceptor (e.g., CAR)) at a weight percentage (w/w) of less than or equal to 40, 35, 30, 25, 20, 19, 18, 17, 16, 15, 14, 13, 12, 11, 10, 9, 8, 7, or 6.5% (and optionally at least 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, or 10%). In some embodiments, an adsorbent is present in a mixture (e.g., dry blended preparation) of NAC and adsorbent or a mixture (e.g., dry blended preparation) of acetyl acceptor (e.g., CAR) and adsorbent (e.g., a first mixture as described herein, pre-treated NAC, or pre-treated acetyl acceptor (e.g., CAR)) at a weight percentage (w/w) of 1-40, 1-35, 1-30, 1-25, 1-20, 1-19, 1-18, 1-17, 1-16, 1-15, 1-14, 1-13, 1-12, 1-11, 1-10, 1-9.5, 1-9, 1-8.5, 1-8, 1-7.5, 1-7, 1-6.5, 1-6.4, 1-6.3, 1-6.2, 1-6.1, 1-6, 1-5.9, 1-5.8, 1-5.7, 1-5.6, 1-5.5, 1-5.4, 1-5.3, 1-5.2, 1-5.1, 1-5, 1-4.5, 1-4, 1-3.5, 1-3, 1-2.5, 1-2, 1-1.5, 2-40, 2-35, 2-30, 2-25, 2-20, 2-19, 2-18, 2-17, 2-16, 2-15, 2-14, 2-13, 2-12, 2-11, 2-10, 2-9.5, 2-9, 2-8.5, 2-8, 2-7.5, 2-7, 2-6.5, 2-6.4, 2-6.3, 2-6.2, 2-6.1, 2-6, 2-5.9, 2-5.8, 2-5.7, 2-5.6, 2-5.5, 2-5.4, 2-5.3, 2-5.2, 2-5.1, 2-5, 2-4.5, 2-4, 2-3.5, 2-3, 2-2.5, 3-40, 3-35, 3-30, 3-25, 3-20, 3-19, 3-18, 3-17, 3-16, 3-15, 3-14, 3-13, 3-12, 3-11, 3-10, 3-9.5, 3-9, 3-8.5, 3-8, 3-7.5, 3-7, 3-6.5, 3-6.4, 3-6.3, 3-6.2, 3-6.1, 3-6, 3-5.9, 3-5.8, 3-5.7, 3-5.6, 3-5.5, 3-5.4, 3-5.3, 3-5.2, 3-5.1, 3-5, 3-4.5, 3-4, 3-3.5, 4-40, 4-35, 4-30, 4-25, 4-20, 4-19, 4-18, 4-17, 4-16, 4-15, 4-14, 4-13, 4-12, 4-11, 4-10, 4-9.5, 4-9, 4-8.5, 4-8, 4-7.5, 4-7, 4-6.5, 4-6.4, 4-6.3, 4-6.2, 4-6.1, 4-6, 4-5.9, 4-5.8, 4-5.7, 4-5.6, 4-5.5, 4-5.4, 4-5.3, 4-5.2, 4-5.1, 4-5, 4-4.5, 5-40, 5-35, 5-30, 5-25, 5-20, 5-19, 5-18, 5-17, 5-16, 5-15, 5-14, 5-13, 5-12, 5-11, 5-10, 5-9.5, 5-9, 5-8.5, 5-8, 5-7.5, 5-7, 5-6.5, 5-6.4, 5-6.3, 5-6.2, 5-6.1, 5-6, 5-5.9, 5-5.8, 5-5.7, 5-5.6, 5-5.5, 5-5.4, 5-5.3, 5-5.2, 5-5.1, 6-40, 6-35, 6-30, 6-25, 6-20, 6-19, 6-18, 6-17, 6-16, 6-15, 6-14, 6-13, 6-12, 6-11, 6-10, 6-9.5, 6-9, 6-8.5, 6-8, 6-7.5, 6-7, 6-6.5, 6-6.4, 6-6.3, 6-6.2, 6-6.1, 7-40, 7-35, 7-30, 7-25, 7-20, 7-19, 7-18, 7-17, 7-16, 7-15, 7-14, 7-13, 7-12, 7-11, 7-10, 7-9.5, 7-9, 7-8.5, 7-8, 7-7.5, 8-40, 8-35, 8-30, 8-25, 8-20, 8-19, 8-18, 8-17, 8-16, 8-15, 8-14, 8-13, 8-12, 8-11, 8-10, 8-9.5, 8-9, 8-8.5, 9-40, 9-35, 9-30, 9-25, 9-20, 9-19, 9-18, 9-17, 9-16, 9-15, 9-14, 9-13, 9-12, 9-11, 9-10, 9-9.5, 10-40, 10-35, 10-30, 10-25, 10-20, 10-19, 10-18, 10-17, 10-16, 10-15, 10-14, 10-13, 10-12, 10-11, 11-40, 11-35, 11-30, 11-25, 11-20, 11-19, 11-18, 11-17, 11-16, 11-15, 11-14, 11-13, 11-12, 12-40, 12-35, 12-30, 12-25, 12-20, 12-19, 12-18, 12-17, 12-16, 12-15, 12-14, 12-13, 13-40, 13-35, 13-30, 13-25, 13-20, 13-19, 13-18, 13-17, 13-16, 13-15, 13-14, 14-40, 14-35, 14-30, 14-25, 14-20, 14-19, 14-18, 14-17, 14-16, 14-15, 15-40, 15-35, 15-30, 15-25, 15-20, 15-19, 15-18, 15-17, 15-16, 16-40, 16-35, 16-30, 16-25, 16-20, 16-19, 16-18, 16-17, 17-40, 17-35, 17-30, 17-25, 17-20, 17-19, 17-18, 18-40, 18-35, 18-30, 18-25, 18-20, 18-19, 19-40, 19-35, 19-30, 19-25, 19-20, 20-40, 20-35, 20-30, 20-25, 25-40, 25-35, 25-30, 30-40, 30-35, or 35-40% (w/w). In some such embodiments, such a mixture, first mixture, or pre-treated NAC comprising NAC does not comprise an acetyl acceptor (e.g., CAR). In some such embodiments, such a mixture, first mixture, or pre-treated acetyl acceptor (e.g., CAR) comprising acetyl acceptor (e.g., CAR) does not comprise NAC.

In some embodiments, the weight ratio of adsorbent to NAC, the weight ratio of adsorbent to acetyl acceptor (e.g., CAR), or the weight ratio of adsorbent to NAC and acetyl acceptor (e.g., CAR) in a mixture (e.g., dry blended preparation) of NAC and adsorbent, acetyl acceptor (e.g., CAR) and adsorbent, or of NAC, acetyl acceptor (e.g., CAR), and adsorbent (e.g., a first mixture as described herein, pre-treated NAC, or pre-treated acetyl acceptor (e.g., CAR)) is less than or equal to 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1.3.6, 1:3.7, 1:3.8, 1:3.9, or 1:4 (and optionally, at least 1:10, 1:9, 1:8, 1:7, 1:6, 1:5, 1:4, 1:3.9, 1:3.8, 1:3.7, 1:3.6, 1:3.5, 1:3.4, 1:3.3, 1:3.2, 1:3.1, 1:3, 1:2.9, 1:2.8, 1:2.7, 1:2.6, 1:2.5, 1:2.4, 1:2.3, 1:2.2, 1:2.1, 1:2, 1:1.9, 1:1.8, 1:1.7, 1:1.6, 1:1.5, 1:1.4, 1:1.3, 1:1.2, or 1:1.1). In some embodiments, the weight ratio of adsorbent to NAC, the weight ratio of adsorbent to acetyl acceptor (e.g., CAR), or the weight ratio of adsorbent to NAC and acetyl acceptor (e.g., CAR) in a mixture (e.g., dry blended preparation) of NAC and adsorbent, acetyl acceptor (e.g., CAR) and adsorbent, or of NAC, acetyl acceptor (e.g., CAR), and adsorbent (e.g., a first mixture as described herein, pre-treated NAC, or pre-treated acetyl acceptor (e.g., CAR)) is at least 1:10, 1:9, 1:8, 1:7, 1:6, 1:5, 1:4, 1:3.9, 1:3.8, 1:3.7, 1:3.6, 1:3.5, 1:3.4, 1:3.3, 1:3.2, 1:3.1, 1:3, 1:2.9, 1:2.8, 1:2.7, 1:2.6, 1:2.5, 1:2.4, 1:2.3, 1:2.2, 1:2.1, 1:2, 1:1.9, 1:1.8, 1:1.7, 1:1.6, 1:1.5, 1:1.4, 1:1.3, 1:1.2, or 1:1.1 (and optionally, less than or equal to 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1.3.6, 1:3.7, 1:3.8, 1:3.9, or 1:4). In some embodiments, the weight ratio of adsorbent to NAC is about 1:1.5, 1:1.6, 1:1.7, 1:1.8, 1:1.9, or 1:2, e.g., about 1:1.7. In some embodiments, the weight ratio of adsorbent to acetyl acceptor (e.g., CAR) is about 1:1, 1:1.1, 1:1.2, 1:1.3, 1:1.4, or 1:1.5, e.g., about 1:1.3. In some embodiments, the weight ratio of adsorbent to NAC and acetyl acceptor (e.g., CAR) is about 1:2.7, 1:2.8, 1:2.9, 1:3, 1:3.1, 1:3.2, or 1:3.3, e.g., about 1:3. In some embodiments, the weight ratio of adsorbent to NAC to acetyl acceptor (e.g., CAR) is about 1:1.7:1.3.

In some embodiments, an adsorbent is present in a dry blended preparation comprising NAC, acetyl acceptor (e.g., CAR), and adsorbent (e.g., a dry blended preparation) at a weight percentage (w/w) of at least 0.05, 0.075, 0.1, 0.125, 0.15, 0.175, 0.2, 0.225, 0.25, 0.275, 0.3, 0.325, 0.35, 0.4, 0.425, 0.45, 0.475, 0.5, 0.6, 0.7, 0.8, 0.9, 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, or 10% (and optionally at less than 10, 9, 8, 7, 6, 5, 4, 3, 2, 1.5, 1, 0.9, 0.8, 0.7, 0.6, 0.575, 0.55, 0.525, 0.5, 0.475, 0.45, 0.425, 0.4, 0.375, 0.35, or 0.325%). In some embodiments, an adsorbent is present in a dry blended preparation comprising NAC, acetyl acceptor (e.g., CAR), and adsorbent (e.g., a dry blended preparation) at a weight percentage (w/w) of less than or equal to 10, 9, 8, 7, 6, 5, 4, 3, 2, 1.5, 1, 0.9, 0.8, 0.7, 0.6, 0.575, 0.55, 0.525, 0.5, 0.475, 0.45, 0.425, 0.4, 0.375, 0.35, or 0.325% (and optionally at least 0.05, 0.075, 0.1, 0.125, 0.15, 0.175, 0.2, 0.225, 0.25, 0.275, 0.3, 0.325, 0.35, 0.4, 0.425, 0.45, 0.475, 0.5, 0.6, 0.7, 0.8, 0.9, 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, or 10%). In some embodiments, an adsorbent is present in a dry blended preparation comprising NAC, acetyl acceptor (e.g., CAR), and adsorbent (e.g., a dry blended preparation) at a weight percentage (w/w) of 0.05-10, 0.05-9.5, 0.05-9, 0.05-8.5, 0.05-8, 0.05-7.5, 0.05-7, 0.05-6.5, 0.05-6.4, 0.05-6.3, 0.05-6.2, 0.05-6.1, 0.05-6, 0.05-5.9, 0.05-5.8, 0.05-5.7, 0.05-5.6, 0.05-5.5, 0.05-5.4, 0.05-5.3, 0.05-5.2, 0.05-5.1, 0.05-5, 0.05-4.5, 0.05-4, 0.05-3.5, 0.05-3, 0.05-2.5, 0.05-2, 0.05-1.5, 0.05-1, 0.05-0.9, 0.05-0.8, 0.05-0.7, 0.05-0.6, 0.05-0.5, 0.05-0.475, 0.05-0.45, 0.05-0.425, 0.05-0.4, 0.05-0.375, 0.05-0.35, 0.05-0.325, 0.05-0.3, 0.05-0.275, 0.05-0.25, 0.05-0.225, 0.05-0.2, 0.05-0.175, 0.05-0.15, 0.05-0.1, 0.05-0.075, 0.075-10, 0.075-9.5, 0.075-9, 0.075-8.5, 0.075-8, 0.075-7.5, 0.075-7, 0.075-6.5, 0.075-6.4, 0.075-6.3, 0.075-6.2, 0.075-6.1, 0.075-6, 0.075-5.9, 0.075-5.8, 0.075-5.7, 0.075-5.6, 0.075-5.5, 0.075-5.4, 0.075-5.3, 0.075-5.2, 0.075-5.1, 0.075-5, 0.075-4.5, 0.075-4, 0.075-3.5, 0.075-3, 0.075-2.5, 0.075-2, 0.075-1.5, 0.075-1, 0.075-0.9, 0.075-0.8, 0.0775-0.7, 0.05-0.6, 0.075-0.5, 0.075-0.475, 0.075-0.45, 0.075-0.425, 0.075-0.4, 0.075-0.375, 0.075-0.35, 0.075-0.325, 0.075-0.3, 0.075-0.275, 0.075-0.25, 0.075-0.225, 0.075-0.2, 0.075-0.175, 0.075-0.15, 0.075-0.1, 0.1-10, 0.1-9.5, 0.1-9, 0.1-8.5, 0.1-8, 0.1-7.5, 0.1-7, 0.1-6.5, 0.1-6.4, 0.1-6.3, 0.1-6.2, 0.1-6.1, 0.1-6, 0.1-5.9, 0.1-5.8, 0.1-5.7, 0.1-5.6, 0.1-5.5, 0.1-5.4, 0.1-5.3, 0.1-5.2, 0.1-5.1, 0.1-5, 0.1-4.5, 0.1-4, 0.1-3.5, 0.1-3, 0.1-2.5, 0.1-2, 0.1-1.5, 0.1-1, 0.1-0.9, 0.1-0.8, 0.1-0.7, 0.1-0.6, 0.1-0.5, 0.1-0.475, 0.1-0.45, 0.1-0.425, 0.1-0.4, 0.1-0.375, 0.1-0.35, 0.1-0.325, 0.1-0.3, 0.1-0.275, 0.1-0.25, 0.1-0.225, 0.1-0.2, 0.1-0.175, 0.1-0.15, 0.2-10, 0.2-9.5, 0.2-9, 0.2-8.5, 0.2-8, 0.2-7.5, 0.2-7, 0.2-6.5, 0.2-6.4, 0.2-6.3, 0.2-6.2, 0.2-6.1, 0.2-6, 0.2-5.9, 0.2-5.8, 0.2-5.7, 0.2-5.6, 0.2-5.5, 0.2-5.4, 0.2-5.3, 0.2-5.2, 0.2-5.1, 0.2-5, 0.2-4.5, 0.2-4, 0.2-3.5, 0.2-3, 0.2-2.5, 0.2-2, 0.2-1.5, 0.2-1, 0.2-0.9, 0.2-0.8, 0.2-0.7, 0.2-0.6, 0.2-0.5, 0.2-0.475, 0.2-0.45, 0.2-0.425, 0.2-0.4, 0.2-0.375, 0.2-0.35, 0.2-0.325, 0.2-0.3, 0.2-0.275, 0.2-0.25, 0.2-0.225, 0.3-10, 0.3-9.5, 0.3-9, 0.3-8.5, 0.3-8, 0.3-7.5, 0.3-7, 0.3-6.5, 0.3-6.4, 0.3-6.3, 0.3-6.2, 0.3-6.1, 0.3-6, 0.3-5.9, 0.3-5.8, 0.3-5.7, 0.3-5.6, 0.3-5.5, 0.3-5.4, 0.3-5.3, 0.3-5.2, 0.3-5.1, 0.3-5, 0.3-4.5, 0.3-4, 0.3-3.5, 0.3-3, 0.3-2.5, 0.3-2, 0.3-1.5, 0.3-1, 0.3-0.9, 0.3-0.8, 0.3-0.7, 0.3-0.6, 0.3-0.5, 0.3-0.475, 0.3-0.45, 0.3-0.425, 0.3-0.4, 0.3-0.375, 0.3-0.35, 0.3-0.325, 0.4-10, 0.4-9.5, 0.4-9, 0.4-8.5, 0.4-8, 0.4-7.5, 0.4-7, 0.4-6.5, 0.4-6.4, 0.4-6.3, 0.4-6.2, 0.4-6.1, 0.4-6, 0.4-5.9, 0.4-5.8, 0.4-5.7, 0.4-5.6, 0.4-5.5, 0.4-5.4, 0.4-5.3, 0.4-5.2, 0.4-5.1, 0.4-5, 0.4-4.5, 0.4-4, 0.4-3.5, 0.4-3, 0.4-2.5, 0.4-2, 0.4-1.5, 0.4-1, 0.4-0.9, 0.4-0.8, 0.4-0.7, 0.4-0.6, 0.4-0.5, 0.4-0.475, 0.4-0.45, 0.4-0.425, 0.5-10, 0.5-9.5, 0.5-9, 0.5-8.5, 0.5-8, 0.5-7.5, 0.5-7, 0.5-6.5, 0.5-6.4, 0.5-6.3, 0.5-6.2, 0.5-6.1, 0.5-6, 0.5-5.9, 0.5-5.8, 0.5-5.7, 0.5-5.6, 0.5-5.5, 0.5-5.4, 0.5-5.3, 0.5-5.2, 0.5-5.1, 0.5-5, 0.5-4.5, 0.5-4, 0.5-3.5, 0.5-3, 0.5-2.5, 0.5-2, 0.5-1.5, 0.5-1, 0.5-0.9, 0.5-0.8, 0.5-0.7, 0.5-0.6, 0.6-10, 0.6-9.5, 0.6-9, 0.6-8.5, 0.6-8, 0.6-7.5, 0.6-7, 0.6-6.5, 0.6-6.4, 0.6-6.3, 0.6-6.2, 0.6-6.1, 0.6-6, 0.6-5.9, 0.6-5.8, 0.6-5.7, 0.6-5.6, 0.6-5.5, 0.6-5.4, 0.6-5.3, 0.6-5.2, 0.6-5.1, 0.6-5, 0.6-4.5, 0.6-4, 0.6-3.5, 0.6-3, 0.6-2.5, 0.6-2, 0.6-1.5, 0.6-1, 0.6-0.9, 0.6-0.8, 0.6-0.7, 0.7-10, 0.7-9.5, 0.7-9, 0.7-8.5, 0.7-8, 0.7-7.5, 0.7-7, 0.7-6.5, 0.7-6.4, 0.7-6.3, 0.7-6.2, 0.7-6.1, 0.7-6, 0.7-5.9, 0.7-5.8, 0.7-5.7, 0.7-5.6, 0.7-5.5, 0.7-5.4, 0.7-5.3, 0.7-5.2, 0.7-5.1, 0.7-5, 0.7-4.5, 0.7-4, 0.7-3.5, 0.7-3, 0.7-2.5, 0.7-2, 0.7-1.5, 0.7-1, 0.7-0.9, 0.7-0.8, 0.8-10, 0.8-9.5, 0.8-9, 0.8-8.5, 0.8-8, 0.8-7.5, 0.8-7, 0.8-6.5, 0.8-6.4, 0.8-6.3, 0.8-6.2, 0.8-6.1, 0.8-6, 0.8-5.9, 0.8-5.8, 0.8-5.7, 0.8-5.6, 0.8-5.5, 0.8-5.4, 0.8-5.3, 0.8-5.2, 0.8-5.1, 0.8-5, 0.8-4.5, 0.8-4, 0.8-3.5, 0.8-3, 0.8-2.5, 0.8-2, 0.8-1.5, 0.8-1, 0.8-0.9, 0.9-10, 0.9-9.5, 0.9-9, 0.9-8.5, 0.9-8, 0.9-7.5, 0.9-7, 0.9-6.5, 0.9-6.4, 0.9-6.3, 0.9-6.2, 0.9-6.1, 0.9-6, 0.9-5.9, 0.9-5.8, 0.9-5.7, 0.9-5.6, 0.9-5.5, 0.9-5.4, 0.9-5.3, 0.9-5.2, 0.9-5.1, 0.9-5, 0.9-4.5, 0.9-4, 0.9-3.5, 0.9-3, 0.9-2.5, 0.9-2, 0.9-1.5, 0.9-1, 1-10, 1-9.5, 1-9, 1-8.5, 1-8, 1-7.5, 1-7, 1-6.5, 1-6.4, 1-6.3, 1-6.2, 1-6.1, 1-6, 1-5.9, 1-5.8, 1-5.7, 1-5.6, 1-5.5, 1-5.4, 1-5.3, 1-5.2, 1-5.1, 1-5, 1-4.5, 1-4, 1-3.5, 1-3, 1-2.5, 1-2, 1-1.5, 2-10, 2-9.5, 2-9, 2-8.5, 2-8, 2-7.5, 2-7, 2-6.5, 2-6.4, 2-6.3, 2-6.2, 2-6.1, 2-6, 2-5.9, 2-5.8, 2-5.7, 2-5.6, 2-5.5, 2-5.4, 2-5.3, 2-5.2, 2-5.1, 2-5, 2-4.5, 2-4, 2-3.5, 2-3, 2-2.5, 3-10, 3-9.5, 3-9, 3-8.5, 3-8, 3-7.5, 3-7, 3-6.5, 3-6.4, 3-6.3, 3-6.2, 3-6.1, 3-6, 3-5.9, 3-5.8, 3-5.7, 3-5.6, 3-5.5, 3-5.4, 3-5.3, 3-5.2, 3-5.1, 3-5, 3-4.5, 3-4, 3-3.5, 4-10, 4-9.5, 4-9, 4-8.5, 4-8, 4-7.5, 4-7, 4-6.5, 4-6.4, 4-6.3, 4-6.2, 4-6.1, 4-6, 4-5.9, 4-5.8, 4-5.7, 4-5.6, 4-5.5, 4-5.4, 4-5.3, 4-5.2, 4-5.1, 4-5, 4-4.5, 5-10, 5-9.5, 5-9, 5-8.5, 5-8, 5-7.5, 5-7, 5-6.5, 5-6.4, 5-6.3, 5-6.2, 5-6.1, 5-6, 5-5.9, 5-5.8, 5-5.7, 5-5.6, 5-5.5, 5-5.4, 5-5.3, 5-5.2, 5-5.1, 6-10, 6-9.5, 6-9, 6-8.5, 6-8, 6-7.5, 6-7, 6-6.5, 6-6.4, 6-6.3, 6-6.2, 6-6.1, 7-10, 7-9.5, 7-9, 7-8.5, 7-8, 7-7.5, 8-10, 8-9.5, 8-9, 8-8.5, 9-10, 9-9.5, or 9.5-10% (w/w).

### Stability

In some embodiments, the methods and compositions described herein increase the stability of NAC in the presence of an acetyl acceptor (e.g., CAR), e.g., by forming a dry blended preparation of NAC and acetyl acceptor (e.g., CAR) under conditions that stabilize NAC, e.g., decrease the deacetylation of NAC. In some embodiments, the conditions comprise contacting NAC or the acetyl acceptor or both with an adsorbent as described herein. In some embodiments, the NAC of a dry blended preparation or amino acid preparation comprising adsorbent has less deacetylation in the presence of acetyl acceptor, e.g., CAR, than NAC in a similar dry blended preparation or amino acid preparation that does not comprise the adsorbent (e.g., as measured by a LC/MS). In some embodiments, the acetyl acceptor, e.g., CAR, of a dry blended preparation or amino acid preparation comprising adsorbent has less acetylation in the presence of NAC than an acetyl acceptor, e.g., CAR, in a similar dry blended preparation or amino acid preparation that does not comprise the adsorbent (e.g., as measured by LC/MS).

In some embodiments, evaluating stability of NAC or acetyl acceptor (e.g., CAR) comprises: providing a dry blended preparation or amino acid preparation comprising NAC and/or acetyl acceptor (e.g., CAR); incubating the dry blended preparation or amino acid preparation for a length of time under selected conditions; and comparing the levels of NAC and/or acetyl acceptor (e.g., CAR) after the incubation to the levels of NAC and/or acetyl acceptor (e.g., CAR) before the incubation. In some embodiments, the provided dry blended preparation or amino acid preparation has been subjected to a downstream processing step such as fill finish and/or packaging. In some embodiments, the dry blended preparation or amino acid preparation is comprised within packaging, e.g., primary packaging or in a final product (e.g., suitable for sale, e.g., to an end user). In some embodiments, at least 90, 91, 92, 93, 94, 95, 96, 97, 98, 98.5, 99, 99.5, or 100% of the NAC of a dry blended preparation, second mixture, or amino acid preparation remains acetylated after 1, 2, 3, 4, 5, 6, 7, 8, 9, 10, 11, 12, 13, 14, 15, 16, 17, 18, 19, 20, 21, 22, 23, or 24 months at 25° C. and 60% relative humidity (e.g., measured as described in Example 6) when the dry blended preparation, second mixture, or amino acid preparation was formed under conditions that stabilize NAC. In some embodiments, at least 80, 81, 82, 83, 84, 85, 86, 87, 88, 89, 90, 90.5, 91, 91.5, 92, 93, 94, 95, 96, 97, 98, 98.5, 99, 99.5, or 100% of the NAC of the dry blended preparation, second mixture, or amino acid preparation remains acetylated after 1, 2, 3, 4, 5, or 6, months at 40° C. and 75% relative humidity (e.g., measured as described in Example 6) when the dry blended preparation, second mixture, or amino acid preparation was formed under conditions that stabilize NAC. In some embodiments, at least 80, 81, 82, 83, 84, 85, 86, 87, 88, 89, 90, 90.5, 91, 91.5, 92, 93, 94, 95, 96, 97, 98, 98.5, 99, 99.5, or 100% of the NAC of the dry blended preparation, second mixture, or amino acid preparation remains acetylated after 1, 2, or 3 months at 60° C. and 75% relative humidity (e.g., measured as described in Example 6) when the dry blended preparation, second mixture, or amino acid preparation was formed under conditions that stabilize NAC.

In some embodiments, the level of NAC (e.g., w/w) in the dry blended preparation, second mixture, or amino acid preparation after 1, 2, 3, 4, 5, 6, 7, 8, 9, 10, 11, 12, 13, 14, 15, 16, 17, 18, 19, 20, 21, 22, 23, or 24 months at 25° C. and 60% relative humidity (e.g., measured as described in Example 6) is at least 90, 91, 92, 93, 94, 95, 96, 97, 98, 98.5, 99, 99.5, or 100% of the original level of NAC in the dry blended preparation, second mixture or amino acid preparation (and optionally, no more than 110, 109, 108, 107, 106, 105, 104, 103, 102, 101, or 100%) when the dry blended preparation, second mixture, or amino acid preparation was formed under conditions that stabilize NAC. In some embodiments, the level of NAC (e.g., w/w) in the dry blended preparation, second mixture, or amino acid preparation after 1, 2, 3, 4, 5, or 6 months at 40° C. and 75% relative humidity (e.g., measured as described in Example 6) is at least 90, 91, 92, 93, 94, 95, 96, 97, 98, 98.5, 99, 99.5, or 100% of the original level of NAC in the dry blended preparation, second mixture or amino acid preparation (and optionally, no more than 110, 109, 108, 107, 106, 105, 104, 103, 102, 101, or 100%) when the dry blended preparation, second mixture, or amino acid preparation was formed under conditions that stabilize NAC. In some embodiments, the level of NAC (e.g., w/w) in the dry blended preparation, second mixture, or amino acid preparation after 1, 2, or 3 months at 60° C. and 75% relative humidity (e.g., measured as described in Example 6) is at least 90, 91, 92, 93, 94, 95, 96, 97, 98, 98.5, 99, 99.5, or 100% of the original level of NAC in the dry blended preparation, second mixture or amino acid preparation (and optionally, no more than 110, 109, 108, 107, 106, 105, 104, 103, 102, 101, or 100%) when the dry blended preparation, second mixture, or amino acid preparation was formed under conditions that stabilize NAC.

In some embodiments, evaluating stability of NAC or acetyl acceptor (e.g., CAR) comprises: providing a dry blended preparation comprising NAC and acetyl acceptor (e.g., CAR); incubating the dry blended preparation for a length of time under selected conditions; and comparing the ratio of the levels of NAC and acetyl acceptor (e.g., CAR) after the incubation to the ratio of the levels of NAC and acetyl acceptor (e.g., CAR) before the incubation. In some embodiments, the ratio of NAC to acetyl acceptor, e.g., CAR, in the dry blended preparation, second mixture, or amino acid preparation is at least 0.1, 0.2, 0.3, 0.4, 0.5, 0.6, 0.7, 0.8, 0.9, 1, 1.2, 1.4, 1.6, 1.8, 2, 2.2, 2.4, 2.6, 2.8, 3, 3.2, 3.4, 3.6, 3.8, or 4 after 1, 2, 3, 4, 5, 6, 7, 8, 9, 10, 11, 12, 13, 14, 15, 16, 17, 18, 19, 20, 21, 22, 23, or 24 months at 25° C. and 60% relative humidity (e.g., measured as described in Example 6) when the dry blended preparation, second mixture, or amino acid preparation was formed under conditions that stabilize NAC. In some embodiments, the ratio of NAC to acetyl acceptor, e.g., CAR, in the dry blended preparation, second mixture, or amino acid preparation is at least 0.1, 0.2, 0.3, 0.4, 0.5, 0.6, 0.7, 0.8, 0.9, 1, 1.2, 1.4, 1.6, 1.8, 2, 2.2, 2.4, 2.6, 2.8, 3, 3.2, 3.4, 3.6, 3.8, or 4 after 1, 2, 3, 4, 5, or 6 months at 40° C. and 75% relative humidity (e.g., measured as described in Example 6) when the dry blended preparation, second mixture, or amino acid preparation was formed under conditions that stabilize NAC. In some embodiments, the ratio of NAC to acetyl acceptor, e.g., CAR, in the dry blended preparation, second mixture, or amino acid preparation is at least 0.1, 0.2, 0.3, 0.4, 0.5, 0.6, 0.7, 0.8, 0.9, 1, 1.2, 1.4, 1.6, 1.8, 2, 2.2, 2.4, 2.6, 2.8, 3, 3.2, 3.4, 3.6, 3.8, or 4 after 1, 2, or 3 months at 60° C. and 75% relative humidity (e.g., measured as described in Example 6) when the dry blended preparation, second mixture, or amino acid preparation was formed under conditions that stabilize NAC. In some embodiments, the ratio of NAC to acetyl acceptor, e.g., CAR, in the dry blended preparation, second mixture, or amino acid preparation is no more than 5, 4.8, 4.6, 4.4, 4.2, 4, 3.8, 3.6, 3.4, 3.2, 3, 2.8, 2.6, 2.4, 2.2, 2, 1.8, 1.6, 1.4, 1.2, or 1 after incubation.

In some embodiments, evaluating stability of NAC or acetyl acceptor (e.g., CAR) comprises: providing a dry blended preparation comprising NAC and acetyl acceptor (e.g., CAR); incubating the dry blended preparation or amino acid preparation for a length of time under selected conditions; and evaluating the level of a NAC and/or acetyl acceptor (e.g., CAR) degradation product after the incubation (and optionally comparing to the level of the NAC and/or acetyl acceptor (e.g., CAR) degradation product before the incubation). In some embodiments, the level of a NAC degradation product (e.g., cysteine, cystine, and/or ALCAR), is less than 1% w/w of the dry blended preparation, second mixture, or amino acid preparation after 1, 2, 3, 4, 5, 6, 7, 8, 9, 10, 11, 12, 13, 14, 15, 16, 17, 18, 19, 20, 21, 22, 23, or 24 months at 25° C. and 60% relative humidity, and/or after 1, 2, 3, 4, 5, or 6 months at 40° C. and 75% relative humidity (e.g., measured as described in Example 6). In some embodiments, a NAC and/or acetyl acceptor (e.g., CAR) degradation product includes, but is not limited to, cysteine (non-acetylated), cystine, or ALCAR.

In some embodiments, the methods and compositions described herein increase the stability of an acetyl acceptor (e.g., CAR) in the presence of NAC, e.g., by forming a dry blended preparation of NAC and acetyl acceptor (e.g., CAR) under conditions that stabilize the acetyl acceptor (e.g., CAR), e.g., decrease the acetylation of CAR. In some embodiments, the conditions that stabilize the acetyl acceptor (e.g., CAR) correspond to the conditions that stabilize NAC described herein. In some embodiments, the acetylation of acetyl acceptor, e.g., CAR, is decreased compared to acetylation of acetyl acceptor (e.g., CAR) in a reference mixture comprising NAC and acetyl acceptor (e.g., CAR) but not formed under conditions that stabilize the acetyl acceptor (e.g., CAR), e.g., in the absence of the adsorbent. In some embodiments, the acetylation of acetyl acceptor, e.g., CAR, is prevented in the presence of an adsorbent (e.g., SiO₂).

Compositions comprising Amino Acid Entities (e.g., Active Moieties)

The composition of the invention as described herein (an Active Moiety) comprises a plurality of amino acid entities, e.g., the amino acid entities shown in Table 1.

In some embodiments, the composition comprises: a) a leucine amino acid entity; b) a arginine amino acid entity; c) glutamine amino acid entity; and d) a N-acetylcysteine (NAC) entity. In some embodiments, the composition comprises: a) a leucine amino acid entity; b) a arginine amino acid entity; c) glutamine amino acid entity; d) a N-acetylcysteine (NAC) entity; e) an isoleucine amino acid entity; and f) a valine amino acid entity.

In some embodiments, the composition comprises: a) a leucine amino acid entity; b) a arginine amino acid entity; c) glutamine amino acid entity; d) a N-acetylcysteine (NAC) entity; e) optionally an isoleucine amino acid entity; and either one or both of f) a carnitine entity or g) a serine amino acid entity.

In some embodiments, the composition comprises: a) a leucine amino acid entity; b) a arginine amino acid entity; c) glutamine amino acid entity; d) a N-acetylcysteine (NAC) entity; e) an isoleucine amino acid entity; f) a valine amino acid entity; and one, two, three, or four of a g) lysine amino acid entity, h) a histidine amino acid entity, i) a phenylalanine amino acid entity, or j) a threonine amino acid entity.

In some embodiments, the composition comprises: a) a leucine amino acid entity, b) a arginine amino acid entity, c) glutamine amino acid entity; d) a NAC entity; and e) a carnitine entity. In some embodiments, the composition further comprises one, two, or more (e.g., all) of (f) a valine amino acid entity, (g) a histidine amino acid entity, or (h) a lysine amino acid entity. In some embodiments, the composition comprises: (a) one or both of a citrulline amino acid entity or an arginine amino acid entity, and (b) a NAC entity. In some embodiments, the composition further comprises one or both of a carnitine entity or a glutamine amino acid entity.

In some embodiments, the composition further comprises one, two, three, four, or more (e.g., all) of a leucine amino acid entity, a histidine amino acid entity, a lysine amino acid entity, a valine amino acid entity, or a serine amino acid entity.

In some embodiments, the composition comprises: a) a leucine amino acid entity, b) an arginine amino acid entity, c) a glutamine amino acid entity, d) a N-acetylcysteine (NAC) entity, and one or both of e) a serine amino acid entity or f) a carnitine entity. In some embodiments, the composition comprises: a) a leucine amino acid entity, b) an arginine amino acid entity, c) a glutamine amino acid entity, d) a N-acetylcysteine (NAC) entity, e) a serine amino acid entity, and f) a carnitine entity. In some embodiments, the composition further comprises an isoleucine amino acid entity.

In some embodiments, the composition comprises: one, two, or all of: a) a leucine amino acid entity; b) an isoleucine amino acid entity; or c) a valine amino acid entity; one, two, or all of: d) a histidine amino acid entity; e) a lysine amino acid entity; or f) a threonine amino acid entity; and one, two, or all of: g) an ornithine amino acid entity; h) an aspartate amino acid entity; or i) a combination salt of an ornithine amino acid entity and an aspartate amino acid entity.

In certain embodiments, the leucine amino acid entity is chosen from L-leucine, β-hydroxy-β-methylbutyrate (HMB), oxo-leucine (a-ketoisocaproate (KIC)), isovaleryl-CoA, n-acetyl-leucine, or a combination thereof.

In certain embodiments, the arginine amino acid entity is chosen from L-arginine, creatine, argininosuccinate, aspartate, glutamate, agmatine, N-acetyl-arginine, or a combination thereof.

In certain embodiments, the glutamine amino acid entity is chosen from L-glutamine, glutamate, carbamoyl-P, N-acetylglutamine, or a combination thereof.

In certain embodiments, the NAC-amino acid entity is chosen from NAC, serine, acetylserine, cystathionine, homocysteine, glutathione, or a combination thereof.

In certain embodiments, the citrulline amino acid entity is chosen from L-citrulline, ornithine, carbamoyl-P, carbamoyl-P and ornithine, N-hydroxyl-arginine, argininosuccinate, nitric oxide, or a combination thereof.

In certain embodiments, the carnitine entity is chosen from L-carnitine, 6-N-trimethyllysine, N6-trimethyl-3-OH-lysine, acetyl-L-carnitine, proprionyl-L-carnitine, L-carnitine L-tartrate, or a combination thereof.

In certain embodiments, the serine amino acid entity is chosen from L-serine, phosphoserine, p-hydroxypyruvate, L-glycine, acetylserine, cystathionine, phosphatidylserine, or a combination thereof.

In certain embodiments, the valine amino acid entity is chosen from L-valine, 2-oxo-valerate, isobutryl-CoA, N-acetyl-valine, or a combination thereof.

In certain embodiments, the histidine amino acid entity is chosen from L-histidine, histidinol, histidinal, ribose-5-phosphate, carnosine, histamine, urocanate, N-acetyl-histidine, or a combination thereof.

In certain embodiments, the lysine amino acid entity is chosen from L-lysine, diaminopimelate, aspartate, trimethyllysine, saccharopine, N-acetyl-lysine, or a combination thereof.

In some embodiments, one, two, three, four, five, six, seven, or more (e.g., all) of the amino acid entities are provided as part of a dipeptide or tripeptide, e.g., in an amount of at least: 0.01 wt. %, 0.1 wt. %, 0.5 wt. %, 1 wt. %, 5 wt. %, or 10 wt. %, or more of amino acid entity components or total components of the composition.

In some embodiments, one, two, three, four, five, six, seven, eight, nine, or more (e.g., all) of the amino acid entities are in free amino acid form in the composition, e.g., at least: 42 wt. %, 45 wt. %, 50 wt. %, 55 wt. %, 60 wt. %, 65 wt. %, 70 wt. %, 75 wt. %, 80 wt. %, 85 wt. %, 90 wt. %, 95 wt. %, 97 wt. %, 98 wt. %, or more, of the total wt. of the composition (e.g., in dry form) is one, two, three, four, five, six, seven, eight, nine, or more (e.g., all) of the amino acid entities in free amino acid form in the composition.

In some embodiments, one, two, three, four, five, six, seven, eight, nine, or more (e.g., all) of the amino acid entities are in salt form in the composition, e.g., at least: 0.01 wt. %, 0.1 wt. %, 0.5 wt. %, 1 wt. %, 2 wt. %, 3 wt. %, 4 wt. %, 5 wt. %, 6 wt. %, 7 wt. %, 8 wt. %, 9 wt. %, or 10 wt. %, or more, of the total wt. of the composition (e.g., in dry form) is one, two, three, four, five, six, seven, eight, nine, or more (e.g., all) of the amino acid entities in salt form in the composition.

In some embodiments, one, two, three, four, five, six, seven, eight, nine, or more (e.g., all) of the amino acid entities is provided as part of a dipeptide or tripeptide, e.g., in an amount of at least: 0.01 wt. %, 0.1 wt. %, 0.5 wt. %, 1 wt. %, 2 wt. %, 3 wt. %, 4 wt. %, 5 wt. %, 6 wt. %, 7 wt. %, 8 wt. %, 9 wt. %, or 10 wt. %, or more, or more of amino acid entity components or total components of the composition.

Exemplary Compositions

An exemplary composition, e.g., dry blended preparation (e.g., comprising an Active Moiety), e.g., PGDBP, can include a combination of amino acid entities disclosed herein, e.g., at a level or ratio disclosed herein, and a wetting agent (e.g., a wetting agent as described herein). An exemplary composition, e.g., dry blended preparation (e.g., comprising an Active Moiety), e.g., PGDBP, can include one or more excipients disclosed herein. In some embodiments, an exemplary composition, e.g., dry blended preparation (e.g., comprising an Active Moiety), e.g., PGDBP, comprises the amino acid entities and/or excipients as described in Tables X1-X6.

In some embodiments, the composition (e.g., the Active Moiety) comprises the amino acids of Table 2 (e.g., at the levels given by Table 2) and a wetting agent (e.g., a wetting agent as described herein).

TABLE 2
Amino acid entities in an exemplary composition

| | Stick Pack | Dry | Single Dose | Daily Dose |
|---|---|---|---|---|
| | Dry | Weight | Dry | Dry |
| Ingredient | Weight (g) | (% w/w) | Weight (g) | Weight (g) |
| L-Leucine | 1.0000 | 11.86 | 4.0000 | 12.0000 |
| L-Isoleucine | 0.5000 | 5.93 | 2.0000 | 6.0000 |
| L-Valine | 0.5000 | 5.93 | 2.0000 | 6.0000 |
| L-Arginine (—HCl) | 1.8140 | 21.52 | 7.2559 | 21.7676 |
| L-Glutamine | 2.0000 | 23.73 | 8.0000 | 24.0000 |
| Acetylcysteine (NAC) | 0.1500 | 1.78 | 0.6000 | 1.8000 |
| AMINO ACIDS (-Salt) | 5.6500 | 67.03 | 22.6000 | 67.8000 |

In some embodiments, the composition (e.g., the Active Moiety) comprises the amino acids of Table 3 (e.g., at the levels given by Table 3) and a wetting agent (e.g., a wetting agent as described herein). In some embodiments, the composition comprises the amino acids of Table 3 and the excipients of Table 3 (e.g., at the levels given by Table 3), and a wetting agent (e.g., a wetting agent as described herein).

TABLE 3
Amino acid entities in an exemplary composition

| | Stick Pack | | Low | High |
|---|---|---|---|---|
| | Dry | Dry | Daily Dose | Daily Dose |
| | Weight | Weight | Dry Weight | Dry Weight |
| Ingredient | (g) | (% w/w) | (g) | (g) |
| L-Leucine | 1.0000 | 13.45 | 4.0000 | 6.0000 |
| L-Isoleucine | 0.5000 | 6.72 | 2.0000 | 3.0000 |
| L-Arginine HCL\hat{ } | 1.6124 | 21.68 | 6.4496 | 9.6744 |
| L-Glutamine | 0.6667 | 8.97 | 2.6666 | 4.0000 |
| L-Serine | 2.5000 | 33.62 | 10.0000 | 15.0000 |
| L-Carnitine | 0.3333 | 4.48 | 1.3333 | 2.0000 |
| Acetylcysteine (NAC) | 0.4333 | 5.83 | 1.7333 | 2.6000 |
| Citric Acid | 0.1972 | 2.65 | 0.7886 | 1.1829 |
| Sucralose | 0.0158 | 0.21 | 0.0631 | 0.0946 |
| Xanthan Gum | 0.0789 | 1.06 | 0.3155 | 0.4732 |
| Vanilla Custard Flavor | 0.0118 | 0.16 | 0.0473 | 0.0710 |
| Orange Flavor | 0.0789 | 1.06 | 0.3155 | 0.4732 |
| FD&C Yellow 6 | 0.0079 | 0.11 | 0.0315 | 0.0473 |
| AMINO ACIDS (—HCL) | 6.7667 | 91.00 | 27.0668 | 40.6002 |
| TOTAL | 7.4361 | 100.00 | 29.7444 | 44.6166 |

In some embodiments, the composition (e.g., the Active Moiety) comprises the amino acids of Table 4 (e.g., at the levels given by Table 4) and a wetting agent (e.g., a wetting agent as described herein).

TABLE 4
Amino acid entities in an exemplary composition

| | Stick Pack | Dry | Single Dose | Daily Dose |
|---|---|---|---|---|
| | Dry | Weight | Dry | Dry |
| Ingredient | Weight (g) | (% w/w) | Weight (g) | Weight (g) |
| L-Leucine | 1.0000 | 11.84 | 4.0000 | 12.0000 |
| L-Isoleucine | 0.5000 | 5.92 | 2.0000 | 6.0000 |
| L-Valine | 0.5000 | 5.92 | 2.0000 | 6.0000 |
| L-Arginine (—HCl) | 1.8140 | 21.47 | 7.2559 | 21.7676 |
| L-Glutamine | 1.3333 | 15.78 | 5.3333 | 16.0000 |
| L-Lysine (-Acetate) | 0.3527 | 4.17 | 1.4108 | 4.2323 |
| L-Histidine | 0.0833 | 0.99 | 0.3333 | 1.0000 |
| L-Phenylalanine | 0.0833 | 0.99 | 0.3333 | 1.0000 |
| L-Threonine | 0.1667 | 1.97 | 0.6667 | 2.0000 |
| Acetylcysteine (NAC) | 0.1500 | 1.78 | 0.6000 | 1.8000 |
| AMINO ACIDS (-Salt) | 5.5667 | 65.89 | 22.2667 | 66.8000 |

In some embodiments, the composition (e.g., the Active Moiety) comprises the amino acids of Table 5 (e.g., at the levels given by Table 5) and a wetting agent (e.g., a wetting agent as described herein). In some embodiments, the composition comprises the amino acids of Table 5 and the excipients of Table 5 (e.g., at the levels given by Table 5), and a wetting agent (e.g., a wetting agent as described herein).

TABLE 5
Amino acid entities and excipients (excipients in italics)

| | | | | High |
|---|---|---|---|---|
| | Stick Pack | Dry | Low Daily | Daily Dose |
| | Dry | Weight | Dose Dry | Dry |
| Ingredient | Weight (g) | (% w/w) | Weight (g) | Weight (g) |
| L-Leucine | 0.8889 | 15.06 | 2.6667 | 8.0000 |
| L-Isoleucine | 0.4444 | 7.53 | 1.3333 | 4.0000 |
| L-Valine | 0.8889 | 15.06 | 2.6667 | 8.0000 |
| L-Lysine Acetate | 0.4703 | 7.97 | 1.4108 | 4.2323 |
| L-Histidine | 0.3333 | 5.65 | 1.0000 | 3.0000 |
| L-Threonine | 0.3333 | 5.65 | 1.0000 | 3.0000 |
| L-Ornithine-L-Aspartate | 1.6667 | 28.23 | 5.0000 | 15.0000 |
| Citric Acid | 0.6000 | 10.16 | 1.8000 | 5.4000 |
| Sucralose | 0.0160 | 0.27 | 0.0480 | 0.1440 |
| Xanthan Gum | 0.0800 | 1.36 | 0.2400 | 0.7200 |
| Vanilla Custard | 0.0200 | 0.34 | 0.0600 | 0.1800 |
| Orange Flavor | 0.1600 | 2.71 | 0.4800 | 1.4400 |
| FD&C Yellow 6 | 0.0016 | 0.03 | 0.0048 | 0.0144 |
| AMINO ACIDS (-Acetate) | 4.8889 | 82.81 | 14.6667 | 44.0000 |
| TOTAL | 5.9034 | 100.00 | 17.7102 | 53.1307 |

In some embodiments, the composition (e.g., the Active Moiety) comprises the amino acids of Table 6 (e.g., at the levels given by Table 6) and a wetting agent (e.g., a wetting agent as described herein). In some embodiments, the composition comprises the amino acids of Table 6 and the excipients of Table 6 (e.g., at the levels given by Table 6), and a wetting agent (e.g., a wetting agent as described herein).

TABLE 6
Amino acid entities and excipients (excipients in italics)

| | Stick | | Single | Daily | Minimum |
|---|---|---|---|---|---|
| | Pack | | Dose | Dose | Single Dose |
| | Dry | Dry | Dry | Dry | Concen- |
| | Weight | Weight | Weight | Weight | tration |
| Ingredient | (g) | (% w/w) | (g) | (g) | (g/L) |
| L-Leucine | 1.000 | 10.29 | 3.00 | 6.00 | 16.91 |
| L-Valine | 0.333 | 3.43 | 1.00 | 2.00 | 5.63 |
| L-Arginine HCL\hat{ } | 2.418 | 24.88 | 7.25 | 14.51 | 40.88 |
| L-Glutamine | 1.667 | 17.15 | 5.00 | 10.00 | 28.18 |
| L-Serine | 0.833 | 8.57 | 2.50 | 5.00 | 14.08 |
| L-Carnitine | 0.333 | 3.43 | 1.00 | 2.00 | 5.63 |
| L-Citrulline | 1.333 | 13.72 | 4.00 | 8.00 | 22.54 |
| L-Lysine HCL\hat{ } | 0.623 | 6.41 | 1.87 | 3.74 | 7.47 |
| L-Histidine | 0.333 | 3.43 | 1.00 | 2.00 | 5.60 |
| Acetylcysteine | 0.433 | 4.46 | 1.30 | 2.60 | 7.32 |
| (NAC) | | | | | |
| Citric Acid | 0.100 | 1.03 | 0.30 | 0.60 | 1.69 |
| Silicon Dioxide | 0.030 | 0.31 | 0.09 | 0.18 | 0.51 |
| Sucralose | 0.016 | 0.16 | 0.048 | 0.096 | 0.27 |
| Lecithin | 0.060 | 0.62 | 0.18 | 0.36 | 1.01 |
| (Alcolec 40P) | | | | | |
| Xanthan Gum | 0.080 | 0.82 | 0.24 | 0.48 | 1.35 |
| Natural Custard | 0.025 | 0.26 | 0.075 | 0.15 | 0.42 |
| Flavor | | | | | |
| Natural Orange | 0.100 | 1.03 | 0.30 | 0.60 | 1.69 |
| Flavor | | | | | |
| FD&C Yellow 6 | 0.0015 | 0.02 | 0.0045 | 0.009 | 0.03 |
| AMINO ACIDS | 8.765 | 90.19 | 26.30 | 52.60 | 148.22 |
| (—HCL) | | | | | |
| TOTAL | 9.719 | 100.00 | 29.16 | 58.31 | 164.34 |

In some embodiments, the composition (e.g., the Active Moiety) comprises the amino acids of Table 7 (e.g., at the levels given by Table 7) and a wetting agent (e.g., a wetting agent as described herein). In some embodiments, the composition comprises the amino acids of Table 7 and the excipients of Table 7 (e.g., at the levels given by Table 7), and a wetting agent (e.g., a wetting agent as described herein).

TABLE 7
Amino acid entities and excipients (excipients in italics)

| | | | Single Dose | Daily Dose |
|---|---|---|---|---|
| | Stick Pack | Dry Weight | Dry Weight | Dry Weight |
| Ingredient | Dry Weight (g) | (% w/w) | (g) | (g) |
| L-Leucine | 1.6667 | 24.17 | 5.0000 | 10.0000 |
| L-Isoleucine | 1.6667 | 24.17 | 5.0000 | 10.0000 |
| L-Valine | 1.6667 | 24.17 | 5.0000 | 10.0000 |
| Acetylcysteine (NAC) | 0.5000 | 7.25 | 1.5000 | 3.0000 |
| Acetyl-L-Carnitine (ALCAR) HCL | 0.2359 | 3.42 | 0.7077 | 1.4153 |
| Citric Acid | 0.6000 | 8.70 | 1.8000 | 3.6000 |
| Sucralose | 0.0480 | 0.70 | 0.1440 | 0.2880 |
| Xanthan Gum | 0.2400 | 3.48 | 0.7200 | 1.4400 |
| Vanilla Custard (Art) | 0.0300 | 0.44 | 0.0900 | 0.1800 |
| Orange (Nat + WONF) | 0.2400 | 3.48 | 0.7200 | 1.4400 |
| FD&C Yellow 6 | 0.0018 | 0.03 | 0.0054 | 0.0108 |
| AMINO ACIDS (—HCL) | 5.7000 | 82.66 | 17.1000 | 34.2000 |
| TOTAL | 6.8957 | 100.00 | 20.6871 | 41.3741 |

In some embodiments, the composition (an Active Moiety) comprises a plurality of amino acid entities, e.g., the amino acid entities shown in Table 8. In some embodiments, the composition comprises: a) a leucine amino acid entity, b) a arginine amino acid entity, c) glutamine amino acid entity; and d) a N-acetylcysteine (NAC) entity. In some embodiments, the composition comprises: a) a leucine amino acid entity, b) a arginine amino acid entity, c) glutamine amino acid entity; d) NAC; and e) CAR. In some embodiments, the composition comprises: a) a leucine amino acid entity, b) a arginine amino acid entity, c) glutamine amino acid entity; d) NAC; and e) a serine amino acid entity and CAR. In some embodiments, the composition further comprises one, two, three or more (e.g., all) of (f) a valine amino acid entity, (g) a histidine amino acid entity, (h) a lysine amino acid entity, or (i) a citrulline amino acid entity. In some embodiments, the composition comprises, consists essentially of, or consists of the amino acids/components of Table 8, e.g., at the g/dose or g/stick pack of Table 8. In some embodiments, the composition comprises one or more excipients, e.g., an adsorbent, in addition to the components listed in Table 8.

TABLE 8
Exemplary composition comprising amino acids (e.g., an Active Moiety).

| | Stick pack (g) | | Total | Wt. | |
|---|---|---|---|---|---|
| Amino Acid | (3 stick packs BID) | Dose (g) | Daily | Ratio | Wt. % |
| L-leucine | 1.0 | 3 | 6 | 3 | 11.41 |
| L-arginine | 2.0 | 6 | 12 | 6 | 22.81 |
| L-glutamine | 1.67 | 5 | 10 | 5 | 19.01 |
| N-acetylcysteine | 0.43 | 1.3 | 2.6 | 1.3 | 4.94 |
| L-carnitine | 0.333 | 1 | 2 | 1 | 3.80 |
| L-serine | 0.83 | 2.5 | 5 | 2.5 | 9.51 |
| L-valine | 0.333 | 1 | 2 | 1 | 3.80 |
| L-histidine | 0.333 | 1 | 2 | 1 | 3.80 |
| L-lysine | 0.5 | 1.5 | 3 | 1.5 | 5.70 |
| L-citrulline | 1.33 | 4 | 8 | 4 | 15.21 |
| Total amino acids | 8.76 | 26.3 | 52.6 | | 100% |

In some embodiments, the composition comprises: a) a leucine amino acid entity, b) an arginine amino acid entity, c) a glutamine amino acid entity, d) NAC, and one or both of e) a serine amino acid entity or f) CAR. In some embodiments, the composition comprises: a) a leucine amino acid entity, b) an arginine amino acid entity, c) a glutamine amino acid entity, d) NAC, e) a serine amino acid entity, and f) CAR. In some embodiments, the composition further comprises an isoleucine amino acid entity. In some embodiments, the composition comprises, consists essentially of, or consists of the amino acids/components of Table 9, e.g., at the g/dose or g/packet of Table 9. In some embodiments, the composition comprises one or more excipients, e.g., an adsorbent, in addition to the components listed in Table 9.

TABLE 9
Exemplary composition comprising amino acids (e.g., an Active Moiety).

| Amino Acid | Packet (g) | Dose (g) | Total Daily | Wt. Ratio | Wt. % |
|---|---|---|---|---|---|
| L-leucine | 1.00 | 3.00 | 6.00 | 3.00 | 14.85 |
| L-isoleucine | 0.50 | 1.50 | 3.00 | 1.50 | 7.43 |
| L-arginine | 1.33 | 4.00 | 8.00 | 4.00 | 19.80 |
| L-glutamine | 0.67 | 2.00 | 4.00 | 2.00 | 9.90 |
| NAC | 0.43 | 1.30 | 2.60 | 1.30 | 6.44 |
| L-carnitine | 0.30 | 0.90 | 1.80 | 0.90 | 4.46 |
| L-serine | 2.50 | 7.50 | 15.00 | 7.50 | 37.13 |
| Total amino acids | 6.73 | 20.20 | 40.40 | 20.20 | 100.00 |

In some embodiments, the composition comprises (a) a leucine amino acid entity; (b) an isoleucine amino acid entity; (c) a valine amino acid entity; (d) a lysine amino acid entity; (e) a histidine amino acid entity; (f) a threonine amino acid entity; (g) an ornithine entity; (h) an aspartate amino acid entity; (i) NAC; and (j) CAR. In some embodiments, the composition comprises, consists essentially of, or consists of the amino acids/components of Table 10, e.g., at the g/day of Table 10. In some embodiments, the composition comprises one or more excipients, e.g., an adsorbent, in addition to the components listed in Table 10.

TABLE 10
Exemplary composition comprising amino acids (e.g., an Active Moiety).

| Amino Acid | Grams/Day or Wt. Ratio |
|---|---|
| L-Leucine | 8 |
| L-Isoleucine | 4 |
| L-Valine | 8 |
| L-Lysine | 3 |
| L-Histidine | 3 |
| L-Threonine | 3 |
| L-Ornithine | 7.5 |
| L-Aspartate | 7.5 |
| N-Acetylcysteine | 1.5-2.5 (e.g., 1.5, 2.0, 2.5) |
| Carnitine | 1.0-2.0 (e.g., 1.0, 1.5, 2.0) |
| Total amino acids | 46.5-48.5 |

In some embodiments, the composition comprises: (a) one or both of a citrulline amino acid entity or an arginine amino acid entity, and (b) NAC. In some embodiments, the composition further comprises one or both of CAR or a glutamine amino acid entity. In some embodiments, the composition further comprises one, two, three, four, or more (e.g., all) of a leucine amino acid entity, a histidine amino acid entity, a lysine amino acid entity, a valine amino acid entity, or a serine amino acid entity.

In certain embodiments, the leucine amino acid entity is chosen from L-leucine, β-hydroxy-β-methylbutyrate (HMB), oxo-leucine (a-ketoisocaproate (KIC)), isovaleryl-CoA, n-acetyl-leucine, or a combination thereof.

In certain embodiments, the arginine amino acid entity is chosen from L-arginine, creatine, argininosuccinate, aspartate, glutamate, agmatine, N-acetyl-arginine, or a combination thereof.

In certain embodiments, the glutamine amino acid entity is chosen from L-glutamine, glutamate, carbamoyl-P, N-acetylglutamine, or a combination thereof.

In certain embodiments, the citrulline amino acid entity is chosen from L-citrulline, ornithine, carbamoyl-P, carbamoyl-P and ornithine, N-hydroxyl-arginine, argininosuccinate, nitric oxide, or a combination thereof.

In certain embodiments, the serine amino acid entity is chosen from L-serine, phosphoserine, p-hydroxypyruvate, L-glycine, acetylserine, cystathionine, phosphatidylserine, or a combination thereof.

In certain embodiments, the glycine amino acid entity is chosen from L-glycine, L-serine, sarcosine, betaine, dimethyglycine, glutathione, creatine, or a combination thereof.

In certain embodiments, the valine amino acid entity is chosen from L-valine, 2-oxo-valerate, isobutryl-CoA, N-acetyl-valine, or a combination thereof.

In certain embodiments, the histidine amino acid entity is chosen from L-histidine, histidinol, histidinal, ribose-5-phosphate, carnosine, histamine, urocanate, N-acetyl-histidine, or a combination thereof.

In certain embodiments, the lysine amino acid entity is chosen from L-lysine, diaminopimelate, aspartate, trimethyllysine, saccharopine, N-acetyl-lysine, or a combination thereof.

In some embodiments, one, two, three, four, five, six, seven, or more (e.g., all) of the amino acid entities are provided as part of a dipeptide or tripeptide, e.g., in an amount of at least: 0.01 wt. %, 0.1 wt. %, 0.5 wt. %, 1 wt. %, 5 wt. %, or 10 wt. %, or more of amino acid entity components or total components of the composition.

In some embodiments, one, two, three, four, five, six, seven, eight, nine, or more (e.g., all) of the amino acid entities are in free amino acid form in the composition, e.g., at least: 42 wt. %, 45 wt. %, 50 wt. %, 55 wt. %, 60 wt. %, 65 wt. %, 70 wt. %, 75 wt. %, 80 wt. %, 85 wt. %, 90 wt. %, 95 wt. %, 97 wt. %, 98 wt. %, or more, of the total wt. of the composition (e.g., in dry form) is one, two, three, four, five, six, seven, eight, nine, or more (e.g., all) of the amino acid entities in free amino acid form in the composition.

In some embodiments, one, two, three, four, five, six, seven, eight, nine, or more (e.g., all) of the amino acid entities are in salt form in the composition, e.g., at least: 0.01 wt. %, 0.1 wt. %, 0.5 wt. %, 1 wt. %, 2 wt. %, 3 wt. %, 4 wt. %, 5 wt. %, 6 wt. %, 7 wt. %, 8 wt. %, 9 wt. %, or 10 wt. %, or more, of the total wt. of the composition (e.g., in dry form) is one, two, three, four, five, six, seven, eight, nine, or more (e.g., all) of the amino acid entities in salt form in the composition.

In some embodiments, one, two, three, four, five, six, seven, eight, nine, or more (e.g., all) of the amino acid entities is provided as part of a dipeptide or tripeptide, e.g., in an amount of at least: 0.01 wt. %, 0.1 wt. %, 0.5 wt. %, 1 wt. %, 2 wt. %, 3 wt. %, 4 wt. %, 5 wt. %, 6 wt. %, 7 wt. %, 8 wt. %, 9 wt. %, or 10 wt. %, or more, or more of amino acid entity components or total components of the composition.

An exemplary dry blended preparation (e.g., comprising an Active Moiety) can include 1.0 g of an leucine amino acid entity; 2.0 g of an arginine amino acid entity; 1.67 g of a glutamine amino acid entity; 0.43 g of NAC; 0.333 g of CAR (e.g., L-carnitine as a free base); 0.83 g of a serine amino acid entity; 0.333 g of a valine amino acid entity; 0.333 g of a histidine amino acid entity; 0.5 g of a lysine amino acid entity; and 1.33 g of a citrulline amino acid entity (see, e.g., g/dose in Table 8).

An exemplary dry blended preparation (e.g., comprising an Active Moiety) can include 1 g of an leucine amino acid entity, 0.5 g of an isoleucine amino acid entity, 1.33 g of an arginine amino acid entity, 0.67g of a glutamine amino acid entity, 0.43 g of NAC, 0.30 g of CAR (e.g., L-carnitine as a free base), and 2.5 of a serine amino acid entity for a total of 6.73 g+/−20% (e.g., g/packet as shown in Table 9).

In some embodiments, the dry blended preparation (e.g., comprising the Active Moiety) includes 1 g+/−20% of an leucine amino acid entity, 0.5 g+/−20% of an isoleucine amino acid entity, 1.33 g+/−20% of an arginine amino acid entity, 0.67 g+/−20% of a glutamine amino acid entity, 0.43 g+/−20% of NAC, 0.30 g+/−20% of CAR (e.g., L-carnitine as a free base), and 2.5 g+/−20% of a serine amino acid entity. In some embodiments, the composition (e.g., the Active Moiety) includes 1 g+/−15% of an leucine amino acid entity, 0.5 g+/−15% of an isoleucine amino acid entity, 1.33 g+/−15% of an arginine amino acid entity, 0.67 g+/−15% of a glutamine amino acid entity, 0.43 g+/−15% of NAC, 0.30 g+/−15% of CAR (e.g., L-carnitine as a free base), and 2.5 g+/−15% of a serine amino acid entity. In some embodiments, the composition (e.g., the Active Moiety) includes 1 g+/−10% of an leucine amino acid entity, 0.5 g+/−10% of an isoleucine amino acid entity, 1.33 g+/−10% of an arginine amino acid entity, 0.67 g+/−10% of a glutamine amino acid entity, 0.43 g+/−10% of NAC, 0.30 g+/−10% of CAR (e.g., L-carnitine as a free base), and 2.5 g+/−10% of a serine amino acid entity. In some embodiments, the composition (e.g., the Active Moiety) includes 1 g+/−5% of an leucine amino acid entity, 0.5 g+/−5% of an isoleucine amino acid entity, 1.33 g+/−5% of an arginine amino acid entity, 0.67 g+/−5% of a glutamine amino acid entity, 0.43 g+/−5% of NAC, 0.30 g+/−5% of CAR (e.g., L-carnitine as a free base), and 2.5 g+/−5% of a serine amino acid entity.

In some embodiments, the composition includes 8 g+/−20% of leucine or the equivalent amount of a leucine amino acid entity, 4 g+/−20% of isoleucine or the equivalent amount of an isoleucine amino acid entity, 8 g+/−20% of valine or the equivalent amount of a valine amino acid entity, 3 g+/−20% of lysine or the equivalent amount of a lysine amino acid entity, 3 g+/−20% of histidine or the equivalent amount of a histidine amino acid entity, 3 g+/−20% of threonine or the equivalent amount of a threonine amino acid entity, 7.5 g+/−20% of ornithine or the equivalent amount of an ornithine amino acid entity, 7.5 g+/−20% of aspartate or the equivalent amount of an aspartate amino acid entity, 1.5 g+/−20%, 2.0 g+/−20%, or 2.5 g+/−20% NAC or the equivalent amount of a NAC entity, and 1.0 g+/−20%, 1.5 g+/−20%, or 2.0 g+/−20% carnitine or the equivalent amount of a carnitine entity (see, e.g., grams/day for composition with NAC and L-carnitine in Table 10).

In some embodiments, a composition, e.g., dry blended preparation, comprises, consists essentially of, or consists of the components listed in Table 11, e.g., in the amounts listed in Table 11. In some embodiments, the composition comprises one or more excipients, e.g., an adsorbent, in addition to or in excess of the amounts of the components listed in Table 11.

TABLE 11
Exemplary composition comprising amino acids (e.g., an Active Moiety).

| | Stick Pack | Stick Pack | Daily Dose |
|---|---|---|---|
| | Dry | Dry Weight | Dry |
| Ingredient | Weight (g) | (% w/w) | Weight (g) |
| L-Arginine HCl | 2.418 | 24.88 | 14.51 |
| L-Citrulline | 1.333 | 13.72 | 8.00 |
| NAC | 0.433 | 4.46 | 2.60 |
| L-Leucine | 1.000 | 10.29 | 6.00 |
| L-Histidine | 0.333 | 3.43 | 2.00 |
| L-Lysine HCl | 0.623 | 6.41 | 3.74 |
| L-Valine | 0.333 | 3.43 | 2.00 |
| L-Serine | 0.833 | 8.57 | 5.00 |
| L-Glutamine | 1.667 | 17.15 | 10.00 |
| L-Carnitine | 0.333 | 3.43 | 2.00 |
| Amino Acids (—HCl): | 8.765 | 90.19 | 52.6 |
| Citric Acid, Anhydrous | 0.100 | 1.03 | 0.60 |
| Silicon Dioxide | 0.030 | 0.31 | 0.18 |
| (Aerosil 300) | | | |
| Lecithin (Alcolec 40P) | 0.060 | 0.62 | 0.36 |
| Xanthan Gum | 0.080 | 0.82 | 0.48 |
| Sucralose | 0.016 | 0.16 | 0.096 |
| Natural Orange Flavor | 0.100 | 1.03 | 0.60 |
| Natural Custard Flavor | 0.025 | 0.26 | 0.15 |
| FD&C Yellow No 6 | 0.0015 | 0.02 | 0.009 |
| Total: | 9.7185 | 100.0 | 58.33 |

In some embodiments, a composition, e.g., dry blended preparation, comprises, consists essentially of, or consists of the components listed in Table 12, e.g., in the amounts listed in Table 12. In some embodiments, the composition comprises one or more excipients, e.g., an adsorbent, in addition to or in excess of the amounts of the components listed in Table 12.

TABLE 12
Exemplary composition comprising amino acids and an adsorbent

| | | | Single | Max Daily | Single |
|---|---|---|---|---|---|
| | Sachet | | Dose | Dose | Dose |
| | Dry | Dry | Dry | Dry | Concen- |
| | Weight | Weight | Weight | Weight | tration |
| Ingredient | (g) | (% w/w) | (g) | (g) | (g/L) in 6 oz.* |
| L-Leucine | 2.0000 | 13.01 | 2.0000 | 12.0000 | 11.27 |
| L-Isoleucine | 1.0000 | 6.51 | 1.0000 | 6.0000 | 5.64 |
| L-Valine | 1.0000 | 6.51 | 1.0000 | 6.0000 | 5.64 |
| L-Arginine HCl | 3.6280 | 23.61 | 3.6280 | 21.7680 | 20.45 |
| L-Glutamine | 4.0000 | 26.03 | 4.0000 | 24.0000 | 22.54 |
| N-Acetyl-L-cysteine (NAC) | 0.3000 | 1.95 | 0.3000 | 1.8000 | 1.69 |
| Citric Acid | 0.6667 | 4.34 | 0.6667 | 4.0002 | 3.76 |
| Lecithin | 1.6666 | 10.85 | 1.6666 | 9.9996 | 9.36 |
| Xanthan Gum | 0.2000 | 1.30 | 0.2000 | 1.2000 | 1.13 |
| Sucralose | 0.0667 | 0.43 | 0.0667 | 0.4002 | 0.38 |
| Orange (Nat + WONF) | 0.3500 | 2.28 | 0.3500 | 2.1000 | 1.97 |
| Vanilla Custard (Art) | 0.0600 | 0.39 | 0.0600 | 0.3600 | 0.34 |
| FD&C Yellow 6 | 0.0090 | 0.06 | 0.0090 | 0.0540 | 0.05 |
| L-HPC (low substituted | 0.1000 | 0.65 | 0.1000 | 0.6000 | 0.56 |
| hydroxypropyl-cellulose) | | | | | |
| Colloidal SiO₂ | 0.3000 | 1.95 | 0.3000 | 1.8000 | 1.69 |
| Magnesium Stearate | 0.0200 | 0.13 | 0.0200 | 0.1200 | 0.11 |
| AMINO ACIDS (—HCL) | 11.3000 | 73.53 | 11.3000 | 67.8000 | 63.68 |
| TOTAL | 15.3670 | 100.00 | 15.3670 | 92.2020 | 86.60 |

*1 sachet is used to form a suspension in 6 oz. of water

In some embodiments, a composition, e.g., dry blended preparation, comprises, consists essentially of, or consists of the components listed in Table 13, e.g., in the amounts listed in Table 13. In some embodiments, the composition comprises one or more excipients, e.g., an adsorbent, in addition to or in excess of the amounts of the components listed in Table 13.

TABLE 13
Exemplary composition comprising amino acids and an adsorbent

| | Sachet | | Single Dose | Max Daily | Single |
|---|---|---|---|---|---|
| | Dry | Dry | Dry | Dose | Dose |
| | Weight | Weight | Weight | Dry Weight | Concentration |
| Ingredient | (g) | (% w/w) | (g) | (g) | (g/L) in 6 oz.* |
| L-Leucine | 2.0000 | 15.15 | 2.0000 | 12.0000 | 16.91 |
| L-Isoleucine | 1.0000 | 7.58 | 1.0000 | 6.0000 | 8.45 |
| L-Valine | 1.0000 | 7.58 | 1.0000 | 6.0000 | 8.45 |
| L-Arginine HCl | 3.6280 | 27.49 | 3.6280 | 21.7680 | 30.67 |
| L-Glutamine | 4.0000 | 30.31 | 4.0000 | 24.0000 | 33.81 |
| N-Acetyl-L-cysteine (NAC) | 0.3000 | 2.27 | 0.3000 | 1.8000 | 2.54 |
| Citric Acid | 0.2000 | 1.52 | 0.2000 | 1.2000 | 1.69 |
| Lecithin | 0.1600 | 1.21 | 0.1600 | 0.9600 | 1.35 |
| Xanthan Gum | 0.2000 | 1.52 | 0.2000 | 1.2000 | 1.69 |
| Sucralose | 0.0300 | 0.23 | 0.0300 | 0.1800 | 0.25 |
| Orange (Nat + WONF) | 0.2000 | 1.52 | 0.2000 | 1.2000 | 1.69 |
| Vanilla Custard (Art) | 0.0500 | 0.38 | 0.0500 | 0.3000 | 0.42 |
| FD&C Yellow 6 | 0.0020 | 0.02 | 0.0020 | 0.0120 | 0.02 |
| L-HPC (low substituted | 0.2640 | 2.00 | 0.2640 | 1.5840 | 2.23 |
| hydroxypropyl-cellulose) | | | | | |
| Colloidal SiO₂ | 0.1320 | 1.00 | 0.1320 | 0.7920 | 1.12 |
| Magnesium Stearate | 0.0330 | 0.25 | 0.0330 | 0.1980 | 0.28 |
| AMINO ACIDS (—HCL) | 11.3000 | 85.61 | 11.3000 | 67.8000 | 95.52 |
| TOTAL | 13.1990 | 100.00 | 13.1990 | 79.1940 | 111.58 |

*1 sachet is used to form a suspension in 6 oz. of water

In some embodiments, a composition, e.g., dry blended preparation, comprises, consists essentially of, or consists of the components listed in Table 14, e.g., in the amounts listed in Table 14. In some embodiments, the composition comprises one or more excipients, e.g., an adsorbent, in addition to or in excess of the amounts of the components listed in Table 14.

TABLE 14
Exemplary composition comprising amino acids and an adsorbent

| | Sachet | | Single Dose | Max Daily | Single |
|---|---|---|---|---|---|
| | Dry | Dry | Dry | Dose | Dose |
| | Weight | Weight | Weight | Dry Weight | Concentration |
| Ingredient | (g) | (% w/w) | (g) | (g) | (g/L) in 6 oz.* |
| L-Leucine | 1.6296 | 15.87 | 3.2592 | 9.7776 | 27.55 |
| L-Isoleucine | 0.8147 | 7.94 | 1.6294 | 4.8882 | 13.77 |
| L-Valine | 1.6296 | 15.87 | 3.2592 | 9.7776 | 27.55 |
| L-Lysine HCl | 0.7631 | 7.43 | 1.5262 | 4.5786 | 12.90 |
| L-Histidine | 0.6111 | 5.95 | 1.2222 | 3.6666 | 10.33 |
| L-Threonine | 0.6111 | 5.95 | 1.2222 | 3.6666 | 10.33 |
| L-Ornithine L-Aspartate | 3.0556 | 29.76 | 6.1112 | 18.3336 | 51.66 |
| Citric Acid | 0.3667 | 3.57 | 0.7334 | 2.2002 | 6.20 |
| Sucralose | 0.0147 | 0.14 | 0.0294 | 0.0882 | 0.25 |
| Xanthan Gum | 0.1467 | 1.43 | 0.2934 | 0.8802 | 2.48 |
| Lecithin | 0.1100 | 1.07 | 0.2200 | 0.6666 | 1.86 |
| Orange (Nat + WONF) | 0.1100 | 1.07 | 0.2200 | 0.6666 | 1.86 |
| FD&C Yellow 6 | 0.0030 | 0.03 | 0.0060 | 0.0180 | 0.05 |
| Colloidal SiO₂ | 0.1694 | 1.65 | 0.3388 | 1.0164 | 2.86 |
| L-HPC (low substituted | 0.2061 | 2.01 | 0.4122 | 1.2366 | 3.48 |
| hydroxypropyl-cellulose) | | | | | |
| Magnesium Stearate | 0.0257 | 0.25 | 0.0514 | 0.1542 | 0.43 |
| AMINO ACIDS (—HCl) | 8.9628 | 87.30 | 17.9256 | 53.7768 | 151.53 |
| TOTAL | 10.2671 | 100.00 | 20.5342 | 61.6026 | 173.59 |

*2 sachets are used to form a suspension in 6 oz. of water

Tables 15 and 16 summarize the single dose concentrations of total amino acids (Table 15) and total hydrophobic amino acids (leucine, isoleucine, valine, phenylalanine, and/or tryptophan) present (Table 16) for the specific compositions listed in Tables 2-14 as a function of the total volume of water the composition is dispersed in. Concentrations are presented both as g/L and g/oz of water for reconstitution.

TABLE 15
Single dose concentrations of total amino acids at constituted volumes

| | | Concentration of total | | | Concentration of total | | |
|---|---|---|---|---|---|---|---|
| Composition | Total | AA (g/L) | | | AA (g/oz) | | |
| (Table #) | AA (g) | in 6 oz | in 4 oz | in 2 oz | in 6 oz | in 4 oz | in 2 oz |
| Table 2 | 22.60 | 127.37 | 191.05 | 382.10 | 3.77 | 5.65 | 11.30 |
| Table 3 | 27.07 | 152.54 | 228.81 | 457.62 | 4.51 | 6.77 | 13.53 |
| Table 4 | 22.27 | 125.49 | 188.23 | 376.46 | 3.71 | 5.57 | 11.13 |
| Table 5 | 14.67 | 82.66 | 123.99 | 247.97 | 2.44 | 3.67 | 7.33 |
| Table 6 | 26.30 | 148.22 | 222.33 | 444.65 | 4.38 | 6.58 | 13.15 |
| Table 7 | 17.10 | 96.37 | 144.56 | 289.11 | 2.85 | 4.28 | 8.55 |
| Table 8 | 26.30 | 148.22 | 222.33 | 444.65 | 4.38 | 6.58 | 13.15 |
| Table 9 | 20.20 | 113.84 | 170.76 | 341.52 | 3.37 | 5.05 | 10.10 |
| Table 11 | 8.77 | 49.40 | 74.10 | 148.19 | 1.46 | 2.19 | 4.38 |
| Table 12 | 11.30 | 63.68 | 95.52 | 191.05 | 1.88 | 2.83 | 5.65 |
| Table 13 | 11.30 | 63.68 | 95.52 | 191.05 | 1.88 | 2.83 | 5.65 |
| Table 14 | 9.11 | 51.37 | 77.05 | 154.10 | 1.52 | 2.28 | 4.56 |

TABLE 16
Single dose concentrations of hydrophobic amino acids at constituted volumes

| | Total | Concentration of total | | | Concentration of total | | |
|---|---|---|---|---|---|---|---|
| Composition | hydrophobic | AA (g/L) | | | AA (g/oz) | | |
| (Table #) | AA (g) | in 6 oz | in 4 oz | in 2 oz | in 6 oz | in 4 oz | in 2 oz |
| Table 2 | 8.00 | 45.09 | 67.63 | 135.26 | 1.33 | 2.00 | 4.00 |
| Table 3 | 6.00 | 33.81 | 50.72 | 101.44 | 1.00 | 1.50 | 3.00 |
| Table 4 | 8.33 | 46.96 | 70.45 | 140.89 | 1.39 | 2.08 | 4.17 |
| Table 5 | 6.67 | 37.57 | 56.36 | 112.71 | 1.11 | 1.67 | 3.33 |
| Table 6 | 4.00 | 22.54 | 33.81 | 67.63 | 0.67 | 1.00 | 2.00 |
| Table 7 | 15.00 | 84.54 | 126.80 | 253.60 | 2.50 | 3.75 | 7.50 |
| Table 8 | 4.00 | 22.54 | 33.81 | 67.63 | 0.67 | 1.00 | 2.00 |
| Table 9 | 4.50 | 25.36 | 38.04 | 76.08 | 0.75 | 1.13 | 2.25 |
| Table 11 | 1.33 | 7.51 | 11.27 | 22.54 | 0.22 | 0.33 | 0.67 |
| Table 12 | 4.00 | 22.54 | 33.81 | 67.63 | 0.67 | 1.00 | 2.00 |
| Table 13 | 4.00 | 22.54 | 33.81 | 67.63 | 0.67 | 1.00 | 2.00 |
| Table 14 | 4.07 | 22.96 | 34.44 | 68.88 | 0.68 | 1.02 | 2.04 |

Amino Acid Molecules to Exclude or Limit from the Composition

In some embodiments, the composition does not comprise a peptide of more than 20 amino acid residues in length (e.g., protein supplement) chosen from or derived from one, two, three, four, five, or more (e.g., all) of egg white protein, soy protein, casein, hemp protein, pea protein, or brown rice protein, or if the peptide is present, the peptide is present at less than: 10 weight (wt.) 5 wt. %, 1 wt. %, 0.1 wt. %, 0.05 wt. %, 0.01 wt. %,of the total wt. of non-amino acid entity protein components or total components in the composition (e.g., in dry form).

In some embodiments, the composition comprises a combination of 3 to 18, 3 to 16, or 3 to 14 different amino acid entities, e.g., the combination comprises at least: 42 wt. %, 75 wt. %, or 90 wt. % of the total wt. % of amino acid entity components or total components in the composition (e.g., in dry form).

In some embodiments, dipeptides or salts thereof or tripeptides or salts thereof are present at less than: 10 wt. %, 0.5 wt. %, 0.1 wt. %, 0.05 wt. %, 0.01 wt. %, 0.001 wt. %, or less of the total wt. of amino acid entity components or total components in the composition (e.g., in dry form).

In some embodiments (e.g., in certain compositions comprising SiO₂), tryptophan is absent from the composition, or if present, is present at less than: 10 wt. %, 9 wt. %, 8 wt. %, 7 wt. %, 6 wt. %, 5 wt. %, 4 wt. %, 3 wt. %, 2 wt. %, 1 wt. %, 0.5 wt. %, 0.1 wt. %, 0.05 wt. %, 0.01 wt. %, 0.001 wt. %, or less, e.g., of the total wt. of the composition (e.g., in dry form). In some embodiments, tryptophan, if present, is present in free form. In some embodiments, tryptophan, if present, is present in salt form. In some embodiments, tryptophan, if present, may be present in an oligopeptide, polypeptide, or protein, with the proviso that the protein is not whey, casein, lactalbumin, or any other protein used as a nutritional supplement, medical food, or similar product, whether present as intact protein or protein hydrolysate. In some embodiments, methionine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, proline is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, one, two, or three of methionine, proline, or tryptophan is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, (e.g., in certain compositions comprising a wetting agent) methionine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, proline is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form). In some embodiments, one or two of methionine or proline is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a carbohydrate (e.g., one, two, three, four, five, six, seven, eight, nine, 10, 11, 12, 13, 14, 15, 16, 17, or 18 of dextrose, maltodextrose, sucrose, dextrin, fructose, galactose, glucose, glycogen, high fructose corn syrup, honey, inositol, invert sugar, lactose, levulose, maltose, molasses, sugarcane, or xylose) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a vitamin (e.g., one, two, three, four, five, six, or seven of vitamin B 1, vitamin B2, vitamin B3, vitamin B6, vitamin B12, vitamin C, or vitamin D) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of nitrate or nitrite are absent from the composition, or if present, are present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, 4-hydroxyisoleucine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a probiotic (e.g., a Bacillus probiotic) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, phenylacetate is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, gelatin (e.g., a gelatin capsule) is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one, two, or three of S-allyl cysteine, S-allylmercaptocysteine, or fructosyl-arginine is absent from the composition, or if present, is present at less than: 10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

Production of Active Moiety and Pharmaceutical Compositions

The present disclosure features a method of manufacturing or making a composition (e.g., an Active Moiety) of the foregoing invention. Amino acid entities used to make the compositions may be agglomerated, and/or instantized to aid in dispersal and/or solubilization.

The compositions may be made using amino acid entities from the following sources, or other sources may used: e.g., FUSI-BCAA™ Instantized Blend (L-Leucine, L-Isoleucine and L-Valine in 2:1:1 weight ratio), instantized L-Leucine, and other acids may be obtained from Ajinomoto Co., Inc. Pharmaceutical grade amino acid entity raw materials may be used in the manufacture of pharmaceutical amino acid entity products. Food (or supplement) grade amino acid entity raw materials may be used in the manufacture of dietary amino acid entity products.

To produce the compositions of the instant disclosure, the following general steps may be used: the starting materials (individual amino acid entities and excipients) may be blended in a blending unit, followed by verification of blend uniformity and amino acid entity content, and filling of the blended powder into stick packs or other unit dosage form. The content of stick packs or other unit dosage forms may be dispersed in water at time of use for oral administration.

Food supplement and medical nutrition compositions of the invention will be in a form suitable for oral administration.

When combining raw materials, e.g., pharmaceutical grade amino acid entities and/or excipients, into a composition, contaminants may be present in the composition. A composition meets a standard for level of contamination when the composition does not substantially comprise (e.g., comprises less than 10, 9, 8, 7, 6, 5, 4, 3, 2, 1, 0.15, 0.1, 0.05, 0.01, or 0.001% (w/w)) a contaminant. In some embodiments, a composition described in a method herein does not comprise a contaminant. Contaminants include any substance that is not deliberately present in the composition (for example, pharmaceutical grade amino acid entities and excipients, e.g., oral administration components, may be deliberately present) or any substance that has a negative effect on a product quality parameter of the composition (e.g., side effects in a subject, decreased potency, decreased stability/shelf life, discoloration, odor, bad taste, bad texture/mouthfeel, or increased segregation of components of the composition). In some embodiments, contaminants include one or more of microbes, metals, e.g., heavy metals, residual solvents or a combination thereof. In some embodiments, the level of contamination, e.g., by one or more of metals e.g., heavy metals, choline, microbes, residual solvents or other contaminants (e.g., contaminants from raw materials) of each portion of a composition is below the level permitted in food.

In some embodiments, a PGDBP of the present invention may be constituted in water to create an aqueous suspension. In some embodiments, the volume of the aqueous suspension is the volume administered to a subject, e.g., a mammalian subject, e.g., a human patient, and contains a single dose of the PGDBP. In some embodiments, the volume of the aqueous suspension is the volume administered to a subject, e.g., a mammalian subject, e.g., a human patient, over a single dosing period (e.g., wherein a dosing period is 1, 2, 3, 4, 5, 6, 7, 8, 9, 10, 11, 12, 18, 24, or 36 hours, or 1, 2, 3, 4, 5, 6, or 7 days, or 1, 2, 3, 4, 5, 6, 7, 8, 9, or 10 weeks). In some embodiments, the volume of the aqueous suspension is a volume described herein.

Excipients

The amino acid compositions of the present disclosure may be compounded or formulated with one or more excipients. Non-limiting examples of suitable excipients include a tastant, a flavorant, a buffering agent, a preservative, a stabilizer, a binder, a compaction agent, a lubricant, a glidant, a dispersion enhancer, a disintegration agent, a flavoring agent, a sweetener, and a coloring agent.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol, ascorbate, ascorbic acid, butylated hydroxytoluene (BHT) and butylated hydroxyanisole and antimicrobials, such as parabens, sorbates, benzoates and sulfites, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the composition comprises a coloring agent. Non-limiting examples of suitable color agents include food, drug and cosmetic colors (FD&C), drug and cosmetic colors (D&C), and natural colors. The coloring agents can be used as dyes or their corresponding lakes.

Particular excipients may include one or more of: citric acid, sweeteners (e.g. sucralose, sucralose micronized NF, acesulfame potassium (e.g. Ace-K)), a dispersion enhancer (e.g. xanthan gum (e.g. Ticaxan Rapid-3)), flavorings (e.g. vanilla custard, e.g., vanilla custard #4306, Orange, e.g., Nat. Orange WONF #1326, lime, e.g., line 865.0032U, and lemon, e.g., lemon 862.2169U), a bitterness masking agent (e.g., bitterness masking agent 936.2160U), and natural or artificial colorings (e.g., FD&C Yellow 6). Exemplary ingredient contents for each stick pack are shown in Table 15.

TABLE 17
Exemplary ingredient contents that may be included in a stick pack

| INGREDIENT | GRADE | FUNCTION | SOURCE; COMMENT |
|---|---|---|---|
| Amino Acids | USP | Active Pharmaceutical | Various sources; Non- |
| | | Ingredient (API) | instantized form (MFG scale) |
| Citric Acid | USP | pH, Flavor | |
| Sucralose | NF | Sweetness (slow onset) | |
| Xanthan Gum | FCC or NF | Stabilizer/Thickener | |
| Vanilla Custard Flavor | GRAS | Taste/Aroma | Mask sulfur |
| Orange Flavor | GRAS | 1° flavor | Citrus profile matches low pH |
| Taste Modifier | GRAS | Bitterness masking | Useful at low volume |
| FD&C Yellow No.6 | FD&C | Color | Match flavor profile |

Production of Dry Blended Preparations

The present disclosure is directed, in part, to methods of improving the dispersion of a hydrophobic amino acid in an aqueous suspension, the method comprising providing a dry blended preparation comprising a hydrophobic amino acid entity (and optionally a wetting agent). To provide, e.g., produce, the dry blended preparations of the instant disclosure, the following general steps may be used: individual pharmaceutical grade amino acid entities (and, optionally, one or more excipients and/or oral administration components), may be combined into a combination and subjected to one or more blending conditions (e.g., blending and mixing). In some embodiments, the blending conditions are continued until the combination meets one or more reference standards. In some embodiments, the resulting PGDBP is divided into a plurality of portions. In some embodiments, at least a percentage of the portions of the plurality of portions also meet one or more reference standards, e.g., the reference standards that the PGDBP met. In some embodiments, at least a percentage of the portions of the plurality of portions meet one or more reference standards.

The present disclosure is further directed, in part, to methods comprising forming a dry blended preparation comprising NAC and an acetyl acceptor (e.g., CAR). To produce the dry blended preparations of the instant disclosure, the following general steps may be used: individual pharmaceutical grade amino acid entities (and, optionally, one or more excipients and/or oral administration components), may be combined into a combination and subjected to one or more blending conditions (e.g., blending and mixing). In some embodiments, the blending conditions are continued until the combination meets one or more reference standards. In some embodiments, the resulting PGDBP is divided into a plurality of portions. In some embodiments, at least a percentage of the portions of the plurality of portions also meet one or more reference standards, e.g., the reference standards that the PGDBP met. In some embodiments, at least a percentage of the portions of the plurality of portions meet one or more reference standards.

In some embodiments, the dry blended preparation, e.g., PGDBP, is also a large-scale preparation. Large-scale, as used herein, describes a preparation that is larger (e.g., by weight, mass, or volume) than a reference value. In some embodiments, the reference value is the size of a typical experimental (e.g., non-manufacturing) preparation. In some embodiments, the reference value is 10, 11, 12, 13, 14, or 15 kg. In some embodiments, large-scale preparations comprise at least 25, 30, 40, 50, 60, 70, 80, 90, 100, 120, 140, 160, 180, 200, 300, 400, 500, 600, 700, 800, 900, 1000, 2000, 3000, 4000, 5000, 6000, 7000, 8000, 9000, or 10000 kg. In some embodiments, large-scale preparations comprise no more than 10000, 5000, 1000, 900, 800, 700, 600, 500, 400, or 300 kg. In some embodiments, a large-scale preparation comprises 100-500 kg, 100-400 kg, 100-300, 100-200 kg, 200-300 kg, 200-400 kg, 200-500 kg, 300-400 kg, 300-500 kg, 400-500, or 500-1000 kg.

Blending Techniques

The methods disclosed herein comprise blending steps which blend and mix combinations of pharmaceutical grade amino acid entities to create PGDBPs that meet a reference standard. Blending conditions used by the methods described herein may utilize any known blending mechanism or combination of blending mechanisms. Blending mechanisms include convection, diffusion, and shear. Convective blending utilizes gross motion of particles, e.g., by gentle rotation within a blender/mixer. Diffusion is the slow, passive blending of particles. Shear blending pushes part of a combination of particles in one direction and another part of the combination of particles in another direction along the same parallel plane. Blending conditions used by the methods described herein may further comprise the use of granulators or other equipment to modify the size and/or shape of particles of combination components (e.g., pharmaceutical grade amino acid entities). In some embodiments, a blending condition comprises inverting and/or shaking a container comprising the dry blended preparation or a subset of ingredients thereof.

In some embodiments, the blending or blending condition employed by a method disclosed herein comprises convective blending. In some embodiments, the blending or blending condition employed by a method disclosed herein comprises diffusion blending. In some embodiments, the blending or blending condition employed by a method disclosed herein comprises shear blending. In some embodiments, the blending or blending condition employed by a method disclosed herein comprises convective and diffusion blending. In some embodiments, the blending or blending condition employed by a method disclosed herein comprises convective and shear blending. In some embodiments, the blending or blending condition employed by a method disclosed herein comprises diffusion and shear blending. In some embodiments, the blending or blending condition employed by a method disclosed herein comprises convective, diffusion, and shear blending.

Blending conditions used by the methods described herein may utilize any known blending or mixing equipment; blending or mixing equipment may operate based on one or more blending mechanisms. There are four main types of blending or mixing equipment: convective, hoppers (i.e., gravimetric), tumblers, and fluidization. In some embodiments, a blending condition or blending step of a method described herein may utilize one or more (e.g., 1, 2, 3, or 4) types of blending or mixing equipment. In some embodiments, dry blended preparations (e.g., PGDBPs) are prepared in batches. In some embodiments, dry blended preparations (e.g., PGDBPs) are prepared in a continuous fashion, e.g., harvesting blended/mixed preparation without interrupting blending or mixing.

The blending or mixing steps of methods disclosed herein are of duration sufficient to produce a dry blended preparation, e.g., PGDBP, which meets a reference standard. In some embodiments, the duration of the blending condition is at least 10, 15, 20, 25, 30, 35, 40, 45, 50, 55, 60, 75, 90, 105, or 120 minutes. In some embodiments, the duration of the blending condition is no more than 180, 165, 150, 135, 120, 105, 90, 75, 60, 55, 50, 45, 40, 35, 30, 25, or 20 minutes. In some embodiments, the duration of the blending condition is 20-90, 20-60, 20-50, 20-40, 20-30, 30-90, 30-60, 30-50, 30-40, 40-90, 40-60, 40-50, 50-90, 50-60, or 60-90 minutes. In some embodiments, the duration of the blending condition is 20-40 minutes, e.g., 20 minutes, 30 minutes, or 40 minutes. In some embodiments, the duration of the blending condition is sufficient such that blending and mixing does not introduce heterogeneity into the combination or dry blended preparation, e.g., by over-mixing. In some embodiments, the duration of the blending condition is determined by evaluation of whether a reference standard has been met. For example, the blending condition may continue until an evaluation shows that the reference standard has been met. In some embodiments wherein the reference standard is composition uniformity, e.g., blend uniformity, evaluating whether a reference standard has been met comprises using near infrared spectroscopy (NIR). In an embodiment, the blending condition is maintained until the NIR spectrum observed shows that a standard for composition uniformity, e.g., blend uniformity, has been met.

In some embodiments, the methods disclosed herein comprise blending steps which blend and mix combinations of pharmaceutical grade amino acid entities to create PGDBPs, wherein the blending steps occur at room temperature, e.g., between 15 and 35° C., e.g., between 20 and 30° C., e.g., at about 25° C. In some embodiments, the blending steps occur at a temperature lower than 20, 21, 22, 23, 24, 25, 26, 27, 28, 29, 30, 31, 32, 33, 34, 35, 36, 37, 38, 39, or 40° C. (and optionally, at a temperature of at least 5, 6, 7, 8, 9, 10, 11, 12, 13, 14, 15, 16, 17, 18, 19, 20, 21, 22, 23, 24, or 25° C.). In some embodiments, the blending steps occur at a temperature of about 20, 21, 22, 23, 24, 25, 26, 27, 28, 29, or 30° C.

In some embodiments, the methods disclosed herein comprise blending steps which blend and mix combinations of pharmaceutical grade amino acid entities to create PGDBPs, wherein the blending steps comprise use of a blender or mixer rotation speed (e.g., a blender or mixer rotor rotational speed) of less than 10,000, 9,000, 8,000, 7,000, 6,000, 5,000, 4,000, 3,000, 2,000, 1,000, 500, 250, 100, 90, 80, 70, 60, 50, 40, 30, 20, or 10 rotations per minute (rpm) (and optionally, at least 5, 10, 15, 20, 25, 30, 35, 40, 45, 50, 60, 70, 80, 90, or 100 rpm). In some embodiments, the blending steps comprise use of a blender or mixer rotation speed (e.g., a blender or mixer rotor rotational speed) of about 20, 30, 40, 50, 60, 70, 80, 90, or 100 rpm. In some embodiments, the blending steps comprise use of a blender or mixer rotation speed (e.g., a blender or mixer rotor rotational speed) of between 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50 rpm.

In some embodiments, the method further comprises roller compaction and/or wet granulation. In some embodiments, the method further comprises automated filling, e.g., which incorporates direct blending, roller compaction, or wet granulation.

Segregation of different species of particles in a combination (e.g., dry blended preparation, e.g., PGDBP) during blending or mixing, division of portions, or downstream processing is a barrier to meeting and maintaining reference standards, e.g., a standard of composition uniformity. Any mixture of two or more types of particles can be vulnerable to segregation. Segregation can occur by one or more of several mechanisms, including sifting, fluidization, and dusting (e.g., see Purutyan, H, and Carson, J. W. Predicting, diagnosing, and solving mixture segregation problems. Jenike & Johnson, CSC Publishing, Powder and Bulk Engineering, 2013).

Sampling and Measurement

The methods described herein for manufacturing a dry blended preparation, e.g., a PGDBP, that meets a reference standard may further comprise evaluating whether the reference standard has been met. In some embodiments, the methods described herein comprise acquiring a value, e.g., for the amount of a pharmaceutical grade amino acid entity, from one or more sampling points in a dry blended preparation, e.g., PGDBP. A sampling point is a location, e.g., defined spatially and temporally, within a dry blended preparation, e.g., PGDBP. In some embodiments, to acquire a value, a sampling point may be accessed. Accessing a sampling point may comprise using a diagnostic technique on the dry blended preparation of the sampling point. In some embodiments, accessing, e.g., using a diagnostic technique, comprises stopping or pausing the blending or mixing or blending condition to access the sampling point. In some embodiments, accessing, e.g., using a diagnostic technique, does not comprise stopping or pausing the blending or mixing or blending condition to access the sampling point. Sampling points may be designated and/or accessed by methods known in the art.

In some embodiments, samples acquired from a sampling point of a combination or dry blended preparation (e.g., PGDBP) or portions of a dry blended preparation (e.g., PGDBP) may be analyzed using near-infrared (NIR) spectroscopy to acquire a value (e.g., for composition uniformity, e.g., blend uniformity). NIR spectroscopy analyzes the absorption spectra of compounds in the NIR wavelength region (780-2500 nm). Absorption peaks of compounds, e.g., pharmaceutical grade amino acid entities, are produced by molecular vibrations classified into two types: overtones and combinations. Compounds comprising CH, OH, or NH bonds can be analyzed using NIR. Methods of interpreting NIR spectra are known in the art. In some embodiments, NIR spectroscopy is used to determine whether the amounts of amino acid entities at a plurality of sampling points are similar, e.g., whether a standard for homogeneity (e.g., composition uniformity, e.g., blend uniformity) has been met. In some embodiments, the methods further comprise, responsive to the determination, selecting and/or executing a step, e.g., selecting and using a blending or mixing technique or blending condition or ending blending, mixing, or a blending condition.

In some embodiments, samples acquired from a sampling point of a combination or dry blended preparation (e.g., PGDBP) or portions of a dry blended preparation (e.g., PGDBP) may be analyzed using high performance liquid chromatography (HPLC, also referred to as high-pressure liquid chromatography) to acquire a value (e.g., for the amount of a pharmaceutical grade amino acid entity).

In some embodiments, samples acquired from a sampling point of a combination or dry blended preparation (e.g., PGDBP) or portions of a dry blended preparation (e.g., PGDBP) may be analyzed using liquid chromatography mass spectrometry (LC-MS). In some embodiments, LC-MS is used to determine the identity and/or amounts of pharmaceutical grade amino acid entities present at a sampling point or in a portion. In some embodiments, LC-MS is used to determine whether a dry blended preparation meets a standard for composition uniformity, e.g., portion or blend uniformity. In some embodiments, the methods further comprise, responsive to the amount(s) of pharmaceutical grade amino acid entities present, selecting and/or executing a step, e.g., selecting and using a blending or mixing technique or blending condition or ending blending, mixing, or a blending condition.

Reference Standards

The methods described herein produce dry blended preparations, e.g., PGDBPs, which meet one or more reference standards. A reference standard, as used herein, means: a standard used or set by:

(1) a manufacturer of a combination (e.g., dry blended preparation, e.g., PGDBP), e.g., a manufacturer having approval from a governmental agency to market the PGDBP, or

(2) the pharmaceutical industry or agencies or entities (e.g., government or trade agencies or entities) regulating the pharmaceutical industry,

to ensure one or more product quality parameters are within acceptable ranges for a medicine, pharmaceutical composition, treatment, or other therapeutic. A product quality parameter can be any parameter regulated by the manufacturer, pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA), Health Canada, and Medicines and Healthcare Products Regulatory Agency (MHRA). A product quality parameter can also be a parameter specified by a national or regional pharmacopeia or formulary, including the U.S. Pharmacopeia (USP), British Pharmacopeia (BP), National Formulary (NF), European Pharmacopeia (EP), Japanese Pharmacopeia (JP), or the International Council for Harmonisation of Technical Requirements for Pharmaceuticals for Human Use (ICH).

The one or more reference standards may be a standard used or promulgated by the pharmaceutical industry or by agencies or entities, e.g., government or trade agencies or entities, regulating the pharmaceutical industry to ensure one or more product quality parameters are within acceptable ranges for a medicine, pharmaceutical composition, treatment, or other therapeutic. The one or more reference standards may be a standard used or set by a manufacturer of a combination (e.g., dry blended preparation, e.g., PGDBP), e.g., a manufacturer having approval from a governmental agency to market the PGDBP, to ensure one or more product quality parameters are within acceptable ranges for a supplement, nutraceutical, medicine, pharmaceutical composition, treatment, or other therapeutic. A product quality parameter can be any parameter regulated by the manufacturer, or by the pharmaceutical industry or by agencies or entities, e.g., government or trade agencies or entities, including but not limited to composition; composition uniformity; dosage; dosage uniformity; presence, absence, and/or level of contaminants or impurities; level of sterility (e.g., the presence, absence and/or level of microbes), color, or particle morphology (e.g., size or shape).

In some embodiments a reference standard comprises a standard of stability. In some embodiments, a standard of stability comprises the level of an amino acid entity, e.g., NAC or an acetyl acceptor (e.g., CAR), (or plurality of amino acid entities, e.g., Active Moiety) varying by less than a threshold amount over a time period and under selected conditions. In some embodiments, a standard of stability comprises the level of an amino acid entity, e.g., NAC or an acetyl acceptor (e.g., CAR), (or plurality of amino acid entities, e.g., Active Moiety) remaining at 90-110% of its original value (e.g., at time 0) over a time period and under selected conditions. In some embodiments, a standard of stability is evaluated by providing a dry blended preparation comprising NAC and acetyl acceptor (e.g., CAR); incubating the dry blended preparation for a length of time under selected conditions; and comparing the ratio of the levels of NAC and acetyl acceptor (e.g., CAR) after the incubation to the ratio of the levels of NAC and acetyl acceptor (e.g., CAR) before the incubation. In some embodiments, the time period is 1, 2, 3, 4, 5, 6, 7, 8, 9, 10, 11, 12, 13, 14, 15, 16, 17, 18, 19, 20, 21, 22, 23, or 24 months. In some embodiments, the selected conditions comprise 25° C. and 60% relative humidity, 40° C. and 75% relative humidity, or 60° C. and 75% relative humidity. In some embodiments, the selected conditions comprise 25° C. and 60% relative humidity or 40° C. and 75% relative humidity. Determining the level of an amino acid entity may comprise any technique described herein, e.g., NIR, LC/MS, and/or HPLC.

Composition Uniformity

In some embodiments, the reference standard is composition uniformity. Composition uniformity, in general, is a standard of homogeneity. Composition uniformity can be classified into two different but related types of uniformity: blend uniformity and portion uniformity (portion uniformity is used interchangeably with content uniformity and dosage uniformity herein). Composition uniformity may comprise one or both types depending on the usage and context. Composition uniformity may comprise a standard of the homogeneity of a combination (e.g., dry blended preparation, e.g., PGDBP) with regards to one or a plurality of components. In some embodiments, a combination that meets a standard for composition uniformity does so with regards to one, two, three, four, or more (e.g., all) components (e.g., pharmaceutical grade amino acid entities).

Blend Uniformity

Blend uniformity refers to the level of homogeneity of the distribution of components in a combination, e.g., dry blended preparation, e.g., PGDBP. In some embodiments, a standard for composition uniformity, e.g., blend uniformity, is met when the amount of a component (e.g., a pharmaceutical grade amino acid entity) at a first sampling point in the combination (e.g., dry blended preparation, e.g., PGDBP) differs by no more than a predetermined amount from a reference value. Amounts may be absolute, e.g., grams, or relative, e.g., weight/weight (e.g., X g of the component in Y g of sampling point). Amounts may be arbitrary values, as in the case of comparing absorbance values to absorbance values or in statistical comparisons of curves, e.g., of spectra. In some embodiments, acquiring a value for blend uniformity comprises assessing a standard for composition uniformity, e.g., blend uniformity, by acquiring a value for the amount of a component at a first sampling point in the combination and comparing it to reference value.

In some embodiments, NIR is used to determine whether the amount of a component (e.g., a pharmaceutical grade amino acid entity) at a first sampling point in the combination (e.g., dry blended preparation, e.g., PGDBP) differs by no more than a predetermined amount from a second or further sampling. Using NIR, the near infrared spectrum for a sampling point can be acquired and compared to the near infrared spectrum for a second or further sampling point (e.g., a third, fourth, fifth, sixth, seventh, eighth, ninth, and/or tenth sampling point) or to the near infrared spectrum for a sample known to meet a reference standard, e.g., a standard for composition uniformity, e.g., blend uniformity. If the comparison shows that the spectra are similar enough to one another, a standard for blend uniformity is met. Similarity of NIR spectra can be evaluated by comparing the conformity index of sampling points. The conformity index is a value generated by the NIR spectra obtained, and the examples of conformity indices described are not an exhaustive list of all possible conformity indices. The conformity index may be the absorbance at a particular wavelength or wavelengths in the near infrared range. The conformity index may be the standard deviation of the average absorbance at a particular wavelength or wavelengths in the near infrared range at a plurality of sampling points. The key characteristic of the conformity index, whichever value is selected, is that the conformity indices of the sampling points accessed converge (in the case of absorbance at particular wavelength) or reduce (in the case of standard deviation) as blending/mixing time increases. For example, the conformity index may be selected to be a wavelength of X nm in the near infrared range. The absorbance at X nm will be measured at a plurality of sampling points at time points during blending. As blending continues, the absorbance at X nm at each sampling point will grow more similar to one another.

In some embodiments, the reference value is the amount of the component at a second or further sampling point (e.g., a third, fourth, fifth, sixth, seventh, eighth, ninth, and/or tenth sampling point) sampling point in the combination (e.g., dry blended preparation, e.g., PGDBP). The second sampling or further sampling point (e.g., a third, fourth, fifth, sixth, seventh, eighth, ninth, and/or tenth sampling point) point may be a different spatial location in the combination,

for example, samples can be collected from a set of predetermined, spread out spatial locations, e.g., a stratified sampling plan with predetermined sites to be sampled, e.g., to obtain samples that represent a variety of locations in the blender or mixer.

In some embodiments, the second sampling point is 1, 2, 3, 4, 5, 6, 7, 8, 9, 10, or more minutes after the first sampling point. In some embodiments, multiple sampling points separated in time are taken throughout the process of manufacturing the dry blended preparation (e.g., PGDBP). In some embodiments, the sampling points separated in time are at intervals throughout the process of manufacturing the dry blended preparation (e.g., PGDBP), e.g., every 1, 2, 3, 4, 5, 6, 7, 8, 9, or 10 minutes. In some embodiments, the multiple sampling points are compared to one another (e.g., the most recent sampling points are compared to each other).

In some embodiments, a standard for composition uniformity, e.g., blend uniformity, is met when the amount of the component at a first sampling point differs from the reference value, e.g., the amount of the component at a second or further sampling point (e.g., a third, fourth, fifth, sixth, seventh, eighth, ninth, and/or tenth sampling point) by less than or equal to 1, 2, 3, 4, 5, 6, 7, 8, 9, or 10%, e.g., 10%. In some embodiments, a standard for composition uniformity is met when the amount of a component at a first sampling differs by no more than 10% from the amount of the component at a second or further sampling point (e.g., a third, fourth, fifth, sixth, seventh, eighth, ninth, and/or tenth sampling point). In some embodiments, a standard for composition uniformity is met when the amount of a component at a first sampling differs by no more than 10% from the amount of the component present in the combination (e.g., dry blended preparation, e.g., PGDBP). In some embodiments, a standard for composition uniformity is met when the amount of a component at the most recent sampling point differs by no more than 10% from the amount of the component present at the next most recent sampling point. Values for the amount of a component present at a sampling point can comprise NIR spectra. Comparisons of values for the amount of a component present at a first, second, or further sampling point can comprise comparison of NIR spectra, e.g., overlaying NIR spectra or comparing conformity indices of the first, second, or further sampling points. Blend uniformity can be met when NIR spectra, e.g., conformity indices, reach a threshold of similarity or overlap.

Portion Uniformity

Portion uniformity refers to the homogeneity of portions of the dry blended preparation, e.g., PGDBP, with respect to amounts of components (e.g., pharmaceutical grade amino acid entities). In some embodiments, the methods described herein comprise division of a dry blended preparation (e.g., PGDBP) into a plurality of portions. In some embodiments, a standard for composition uniformity, e.g., portion uniformity, is met when the amount of a component (e.g., a pharmaceutical grade amino acid entity) in a first portion differs by no more than a predetermined amount from a reference value. Amounts may be absolute, e.g., grams, or relative, e.g., weight/weight (e.g., X g of the component in Y g of sampling point). In some embodiments, the amount of a component (e.g., a pharmaceutical grade amino acid entity) in a first, second, or further portion (e.g., a third, fourth, fifth, sixth, seventh, eighth, ninth, or tenth portion) is determined using HPLC.

In some embodiments, the reference value is the amount of the component in a second portion. In some embodiments, the reference value is the amount(s) of the component in a plurality of portions, e.g., a plurality of test portions (e.g., the first portion is compared to a plurality of test portions). In an embodiment, the reference value is the average or median amount of the component in the plurality of test portions.

In some embodiments, a standard for composition uniformity, e.g., portion uniformity, is met when the amounts of a component (e.g., a pharmaceutical grade amino acid entity) in a plurality of test portions differ by no more than a predetermined amount from a reference value. Amounts may be absolute, e.g., grams, or relative, e.g., weight/weight (e.g., X g of the component in Y g of sampling point). In some embodiments, the reference value is the average or median amount of the component in the plurality of test portions.

In some embodiments, the reference value is the amount of the component in the combination (e.g., dry blended preparation, e.g., PGDBP). For example, the reference value can be overall weight/weight of the component present in the total combination. In some embodiments, evaluating whether a standard for composition uniformity is met comprises comparing a relative amount of a component at a first sampling point (e.g., X g of the component in Y g of sampling point) to the relative amount of the component in the combination (e.g., W g of the component in Z g of combination total); in other words, evaluating the standard for composition uniformity may comprise comparing X/Y to W/Z.

In an embodiment, at least X% of the portions of the plurality of portions of the dry blended preparation (e.g., PGDBP) are test portions, wherein X is 1, 2, 3, 4, 5, 6, 7, 8, 9, 10, 15, 20, 25, 30, 35, 40, 45, or 50. In some embodiments, no more than X% of the portions of the plurality of portions of the dry blended preparation (e.g., PGDBP) are test portions, wherein X is 50, 45, 40, 35, 30, 25, 20, 15, 10, 9, 8, 7, 6, 5, 4, 3, 2, or 1. In an embodiment, test portions are portions compared to a reference value, e.g., one another or the amount of a component present in the dry blended preparation (e.g., PGDBP), to determine whether a reference standard (e.g., for composition uniformity, e.g., portion uniformity) has been met. In some embodiments, a standard for composition uniformity, e.g., portion uniformity, is met when the amount of a component present in at least X% of test portions differs from a reference value by no more than 1, 2, 3, 4, 5, 6, 7, 8, 9, or 10%, wherein X is 50, 60, 70, 80, 85, 90, 95, 99, or 100%, and wherein the reference value is selected from the average amount of the component present in the test portions, the median amount of the component present in the test portions, or the amount of the component present in the dry blended preparation (e.g., PGDBP).

In some embodiments, portions of the dry blended preparation (e.g., PGDBP) may be stick packs or other unit dosage forms.

Suspension Uniformity

In some embodiments, a dry blended preparation (e.g., PGDBP) or an aqueous suspension comprising a dry blended preparation meets a reference standard. In some embodiments, the reference standard is suspension uniformity. Suspension uniformity, in general, is a standard of homogeneity in an aqueous suspension (e.g., a standard of homogeneity of a dry blended preparation in an aqueous suspension). Suspension uniformity may comprise a standard of the homogeneity of an aqueous suspension, e.g., comprising a dry blended preparation and water, with regards to one or a plurality of components. In some embodiments, an aqueous suspension that meets a standard for suspension uniformity does so with regards to one, two, three, four, or more (e.g., all) components (e.g., pharmaceutical grade amino acid entities), e.g., of a dry blended preparation.

Without wishing to be bound by theory, certain components of a dry blended preparation may not completely solubilize or disperse upon combining with water, and more efficient and/or complete dispersion of components of a dry blended preparation constituted into an aqueous suspension may be desirable as it improves characteristics of the aqueous suspension (e.g., taste, mouthfeel, and/or dose delivery). A dry blended preparation (e.g., PGDBP) or aqueous suspension comprising the same that meets a standard for suspension uniformity may exhibit improved dispersion of components of the dry blended preparation and, accordingly, may have improved taste, mouthfeel, and/or dose delivery.

Suspension uniformity, in part, refers to the level of homogeneity of the distribution of one or more components in an aqueous suspension, e.g., an aqueous suspension comprising a dry blended preparation, e.g., PGDBP, and water. In some embodiments, a standard for suspension uniformity is met when the amount of a component (e.g., a pharmaceutical grade amino acid entity) at a first sampling point in the aqueous suspension differs by no more than a predetermined amount from a reference value. Amounts may be absolute, e.g., grams, or relative, e.g., weight/weight (e.g., X g of the component in Y g of sampling point). Amounts may be arbitrary values, as in the case of comparing absorbance values to absorbance values or in statistical comparisons of curves, e.g., of spectra. In some embodiments, acquiring a value for suspension uniformity comprises assessing a standard for suspension uniformity by acquiring a value for the amount of a component at a first sampling point in the combination and comparing it to reference value.

In some embodiments, HPLC-UV, UPLC-UV, OPA tagged HPLC-UV, Accqtag HPLC-UV, and/or LC/MS may be used to determine whether the amount of a component (e.g., a pharmaceutical grade amino acid entity) at a first sampling point in the aqueous suspension differs by no more than a predetermined amount from a second or further sampling. Using one of said techniques, the identities and/or levels of one or more components present at a sampling point can be acquired and compared to the identities and/or levels of one or more components at a second or further sampling point (e.g., a third, fourth, fifth, sixth, seventh, eighth, ninth, and/or tenth sampling point) or to the identities and/or levels of one or more components in a sample known to meet a reference standard, e.g., a standard for suspension uniformity. If the comparison shows that the identities and/or levels of the one or more components are similar enough to one another, a standard for suspension uniformity is met. Alternately, if the comparison shows that the identities and/or levels of the one or more components are similar enough to a reference value (e.g., the level of component expected in an aqueous suspension (e.g., of that volume of liquid and total amount of that component) that meets a standard of suspension uniformity). In some embodiments, if the level of a component is within 90-110% of the level of a component in the second or further sampling point or from the reference value (e.g., the average amount of that component expected to be present in the aqueous suspension) then the aqueous suspension meets a standard for suspension uniformity (e.g., with respect to that component).

In some embodiments, the reference value is the amount of the component at a second or further sampling point (e.g., a third, fourth, fifth, sixth, seventh, eighth, ninth, and/or tenth sampling point) sampling point in the aqueous suspension. The second sampling or further sampling point (e.g., a third, fourth, fifth, sixth, seventh, eighth, ninth, and/or tenth sampling point) point may be a different spatial location in the aqueous suspension, for example, samples can be collected from a set of predetermined, spread out spatial locations, e.g., a stratified sampling plan with predetermined sites to be sampled, e.g., to obtain samples that represent a variety of locations in the container, blender, or mixer.

In some embodiments, the second sampling point is 1, 2, 3, 4, 5, 6, 7, 8, 9, 10, or more minutes after the first sampling point. In some embodiments, multiple sampling points separated in time are taken throughout the process of constituting a dry blended preparation (e.g., PGDBP) into an aqueous suspension. In some embodiments, the sampling points separated in time are at intervals throughout the process of constituting the dry blended preparation (e.g., PGDBP) into an aqueous suspension, e.g., every 1, 2, 3, 4, 5, 6, 7, 8, 9, 10, 15, 20, 25, 30, 35, 40, 45, 50, 55, or 60 minutes. In some embodiments, the multiple sampling points are compared to one another (e.g., the most recent sampling points are compared to each other).

In some embodiments, a standard for suspension uniformity is met when the amount of the component at a first sampling point differs from the reference value, e.g., the amount of the component at a second or further sampling point (e.g., a third, fourth, fifth, sixth, seventh, eighth, ninth, and/or tenth sampling point) by less than or equal to 1, 2, 3, 4, 5, 6, 7, 8, 9, or 10%, e.g., 10%. In some embodiments, a standard for suspension uniformity is met when the amount of a component at a first sampling differs by no more than 10% from the amount of the component at a second or further sampling point (e.g., a third, fourth, fifth, sixth, seventh, eighth, ninth, and/or tenth sampling point). In some embodiments, a standard for suspension uniformity is met when the amount of a component at a first sampling differs by no more than 10% from the amount of the component present in the aqueous suspension (e.g., present in the dry blended preparation, e.g., PGDBP, comprised in the aqueous suspension). In some embodiments, a standard for suspension uniformity is met when the amount of a component at the most recent sampling point differs by no more than 10% from the amount of the component present at the next most recent sampling point. Values for the amount of a component present at a sampling point can comprise LC/MS and/or HPLC data or levels or identities obtained therefrom. Comparisons of values for the amount of a component present at a first, second, or further sampling point can comprise comparison of LC/MS and/or HPLC data. Suspension uniformity can be met when LC/MS and/or HPLC data show a similarity in concentration values (e.g., uniformity of dosage unit). Additionally or alternatively, suspension uniformity can be met using light scattering intensity-based techniques when scattering intensity at a specified wavelength (e.g. at 600 nm) does not change across time.

Suspension uniformity, in part, also may refer to the level of homogeneity of the distribution of one or more components in an aqueous suspension over time, e.g., floating or sedimentation. Without wishing to be bound by theory, after constituting or reconstituting a dry blended preparation into an aqueous suspension, a component of the aqueous suspension (e.g., a hydrophobic amino acid entity) may float to the surface over time or remain at the surface even after vigorous shaking. Inclusion of a wetting agent in an aqueous suspension or dry blended preparation for use in an aqueous suspension may stabilize a component in an aqueous suspension, e.g., decrease the rate at which the component floats to the surface of the suspension. In some embodiments, a standard for suspension uniformity is met when the amount of a component (e.g., a pharmaceutical grade amino acid entity) at a first sampling time point in the aqueous suspension differs by no more than a predetermined amount from the amount of the component at a second later sampling time point, e.g., wherein the predetermined amount is 1, 2, 3, 4, 5, 6, 7, 8, 9, or 10% of the level of the component present at the first sampling point in time. In some embodiments, the first sampling time point and second sampling time point are at least 1, 2, 3, 4, 5, 6, 7, 8, 9, 10, 15, 20, 25, 30, 45, or 60 seconds, or 1, 2, 3, 4, 5, 6, 7, 8, 9, 10, 15, 20, 30, 40, 50, or 60 minutes apart. In some embodiments, an aqueous suspension or dry blended preparation for use in, produced by, or modified by a method described herein meets a standard of suspension uniformity with regards to sedimentation when the first sampling time point and second sampling time point are at least 1, 2, 3, 4, 5, 6, 7, 8, 9, 10, 15, 20, 25, 30, 45, or 60 seconds, or 1, 2, 3, 4, 5, 6, 7, 8, 9, 10, 15, 20, 30, 40, 50, or 60 minutes apart.

Level of Contamination

In some embodiments, the reference standard is level of contamination. When combining raw materials, e.g., pharmaceutical grade amino acid entities and/or excipients, into a combination, e.g., dry blended preparation, e.g., PGDBP, contaminants may be present in the combination. A combination, e.g., dry blended preparation, e.g., PGDBP, meets a standard for level of contamination when the combination does not substantially comprise (e.g., comprises less than 10, 9, 8, 7, 6, 5, 4, 3, 2, 1, 0.15, 0.1, 0.05, 0.01, or 0.001% (w/w) of) a contaminant. In some embodiments, a combination, e.g., dry blended preparation, e.g., PGDBP, comprises less than 0.15% (w/w) of a contaminant. In some embodiments, a combination, e.g., dry blended preparation, e.g., PGDBP, comprises a lower level of a contaminant than the level permissible in food (e.g., as defined by appropriate regulatory organizations known in the art). In some embodiments, a combination, e.g., dry blended preparation, e.g., PGDBP, described in a method herein does not comprise a contaminant. Contaminants include any substance that is not deliberately present in the combination, e.g., dry blended preparation, e.g., PGDBP, (for example, pharmaceutical grade amino acid entities and excipients, e.g., oral administration components, are deliberately present) or any substance that has an unintended negative effect on a product quality parameter of the PGDBP or plurality of portions of PGDBP (e.g., side effects in a subject, decreased potency, decreased stability/shelf life, discoloration, odor, bad taste, bad texture/mouthfeel, or increased segregation of components of the PGDBP). In some embodiments, contaminants include microbes, endotoxins, metals (e.g., heavy metals), residual solvents, raw material impurities, extractables, and/or leachables. In some embodiments, a combination, e.g., dry blended preparation, e.g., PGDBP, comprises a level of contaminant (e.g., does not substantially comprise a contaminant) that is compliant with a reference standard, e.g., a standard promulgated by an agency known to those of skill in the art or described herein. In some embodiments, a combination, e.g., dry blended preparation, e.g., PGDBP, comprises a level of contaminant (e.g., does not substantially comprise a contaminant) that is compliant with a standard of the ICH, e.g., the ICH Q3A Impurities in New Drug Substances standard.

In some embodiments, the methods described herein further comprise acquiring a value for the level of a contaminant at a sampling point in one or both of the combination or PGDBP. In some embodiments, the methods described herein further comprise acquiring a value for the level of a contaminant at each of a plurality of points in one or both of the combination or PGDBP, or in a test portion (e.g., of the combination or PGDBP). In some embodiments, the methods described herein further comprise acquiring a value for the level of a contaminant in a portion, e.g., a test portion, of the plurality of portions. In some embodiments, responsive to the value for the level of the contaminant, e.g., and determining that a standard for the level of contamination is met, the methods described herein further comprise selecting and executing a downstream processing step, e.g., dividing the PGDBP into portions (e.g., portioning) and fill-finish (e.g., formulation (e.g., with excipients), packaging, and labeling) and distribution. In some embodiments, responsive to the value for the level of the contaminant, e.g., and determining that a standard for the level of contamination is not met, the methods described herein further comprise selecting and executing a different downstream processing step, e.g., purification and/or removal of the contaminant or disposal of the portion, plurality of portions, or PGDBP.

Dietary Compositions

The composition (e.g., Active Moiety) including amino acid entities can be formulated and used as a dietary composition, e.g., chosen from a medical food, a functional food, or a supplement. In such an embodiment, the raw materials and final product should meet the standards of a food product. In some embodiments, the dietary composition is for use in a method, comprising administering the composition to a subject.

In some embodiments, the dietary composition is chosen from a medical food, a functional food, or a supplement. In some embodiments, the composition is in the form of a nutritional supplement, a dietary formulation, a functional food, a medical food, a food, or a beverage comprising a composition described herein.

In some embodiments, the compositions comprising the active moieties described herein are used in non-therapeutic ways, such as to provide nutrition, maintain health, or improve cosmetic appearance in healthy subjects. Examples include, but are not limited to, use of the compositions described herein as dietary supplements or as food.

### EXAMPLES

The Examples below are set forth to aid in the understanding of the inventions, but are not intended to, and should not be construed to, limit its scope in any way.

### Example 1

### Testing Wetting Agents in Aqueous Suspensions of Exemplary Dry Blended Preparation 1

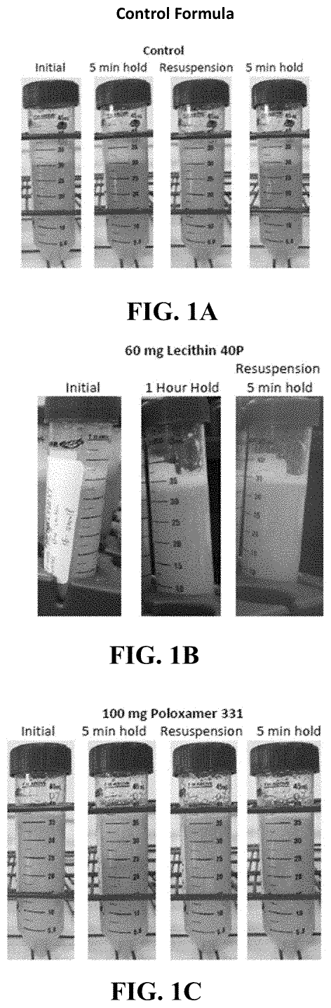

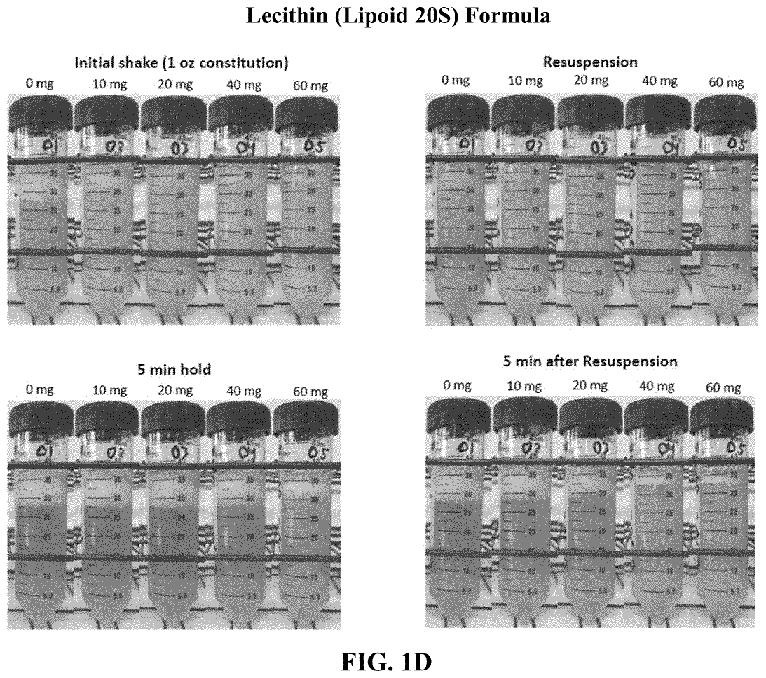

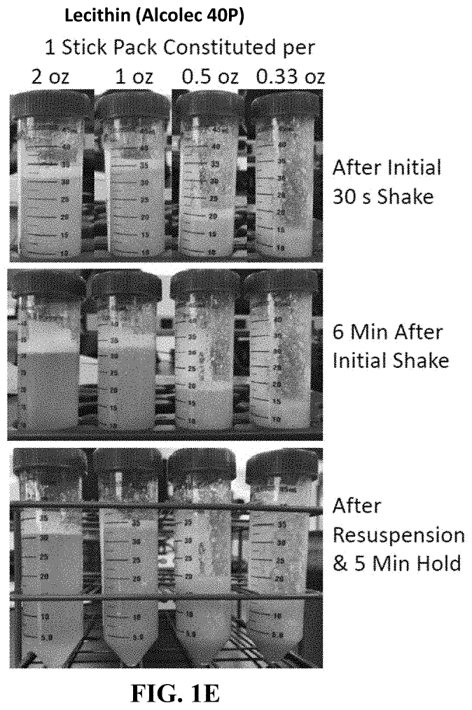

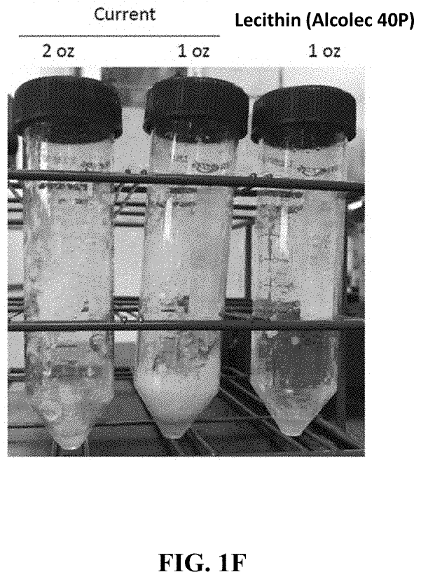

This Example describes the evaluation of the dispersion of amino acid entities in aqueous suspensions comprising exemplary dry blended preparation 1 (comprising an Active Moiety) using various wetting agents, determining whether a wetting agent improves dispersion of hydrophobic amino acids, e.g., suspension uniformity and/or foaming. First, the amino acid entities and additional components listed in Table 18 (columns 1-3), were mixed into a single dose of exemplary dry blended preparation 1. Control doses were constituted into 30 mL (1 ounce) water to create an aqueous suspension without wetting agent, whereas test doses of dry blended preparation had either lecithin (Alcolec 40P) included in the dry blended preparation or poloxamer 331 included in the water used to constitute the aqueous suspension. The aqueous suspension was steadily shaken for at least 30 seconds. As can be seen in FIGS. 1A-1C, photographs were taken for comparison, immediately following the 30 second shake, 5 minutes of holding post-shaking, after resuspension performed at 5 minutes of holding post-shaking, 5 minutes of holding post-resuspension, 1 hour of holding post-shaking, and/or after resuspension performed at 1 hour of holding post-shaking, to visually determine by eye, if the aqueous suspension had achieved a standard for suspension uniformity and/or the extent to which foaming (the layer of foam) had resolved.

As shown in FIG. 1A, the control aqueous suspension that contained no additional wetting agent did not display uniform mixing and also led to the formation of foam which traps the hydrophobic amino acids and prevents them from dispersing uniformly into the liquid. The inclusion of wetting agents, lecithin (Alcolec 40P) (FIG. 1B) or poloxamer 331 (FIG. 1C), greatly enhanced amino acid entity dispersion compared to the control (no wetting agent), e.g., as seen by the smaller foam layer and more rapid decrease in the foam layer and little to no floating hydrophobic amino acids in the foam layer.

A singular dose of exemplary dry blended preparation 1 was prepared containing increasing amounts of the wetting agent lecithin (Lipoid 20S), ranging from 0, 10 mg, 20 mg, 40 mg, and 60 mg, as summarized in Table 18 (column 4) and was added to one ounce of water. Photographs after the initial 30 second shake, 5 minutes of holding post-initial shaking, immediately after resuspension, and 5 minutes of holding following resuspension (FIG. 1D). As shown in FIG. 1D, Lecithin (Lipoid 20S) was able to increase amino acid entity dispersion at the various concentrations investigated, resulting in a relatively uniform suspension after 5 minutes of holding following shaking. Increasing the concentration of wetting agent improved dispersion and reduced the foam layer size and the time it took for the foam layer to decrease in size and greatly reduced the hydrophobic amino acids trapped in the foam layer at the surface.

The ability of lecithin (Alcolec 40P) to enhance the dispersion of amino acid entities of a single dose of exemplary dry blended preparation 1 in aqueous suspensions of different volumes was investigated (FIG. 1E). A single dose of exemplary dry blended preparation 1 was mixed with 60 mg of lecithin (Alcolec 40P) and then added to 2, 1, 0.5 or 0.33 ounces of water. Dispersion was evaluated by eye immediately after 30 seconds shaking, after 6 minutes of holding after shaking, and 5 minutes of holding after resuspension. As shown in FIG. 1E, constitution of exemplary dry blended preparation 1 with the wetting agent at lower volumes than one ounce also led to an improved dispersion (e.g., reduced foaming and reduced amount of hydrophobic amino acids trapped in foam) relative to non-wetting agent containing suspensions. In order to replicate dosing, the one ounce aqueous suspension comprising exemplary dry blended preparation 1 and 60 mg of lecithin (Alcolec 40P) or the one and two ounce aqueous suspensions of exemplary dry blended preparation 1 without a wetting agent were decanted, and the amount of non-dispersed material that remained on the inside of the tube was used as a measure of dispersion efficiency and delivery of amino acid entities (e.g., Active Moiety). The less non-dispersed material remaining on the side of the tube, the better the dispersion, and thus the greater the dose of the dry blended preparation delivered to a hypothetical subject. As shown in FIG. 1F, the aqueous suspension including lecithin (Alcolec 40P) as a wetting agent greatly improved the dispersion compared to controls with no wetting agents. This is evidenced by the very little non-dispersed material on sides of the tube following decanting.

These data indicate that wetting agent, e.g., lecithin (Alcolec 40P), poloxamer 331, and lecithin (Lipoid 20S), improves the dispersion of amino acid entities in aqueous suspensions of exemplary dry blended preparation 1, and this when included in such aqueous suspensions may allow for improved dispersion of hydrophobic amino acid entities and consequently improved dose delivery to a subject.

TABLE 18
Components of exemplary dry blended preparation 1 with different wetting agents (lecithin (Alcolec
40P), poloxamer 331, and lecithin (Lipoid 20S)) investigated for improving dispersion

| Control | | Lecithin | | | | Lecithin | |
|---|---|---|---|---|---|---|---|
| (no wetting agent) | | (Alcolec 40P) | | Poloxamer 331 | | (Lipoid 20S) | |
| Material | g/dose | Material | g/dose | Material | g/dose | Material | g/dose |
| L-Leucine | 0.889 | L-Leucine | 0.889 | L-Leucine | 0.889 | L-Leucine | 0.889 |
| L-Isoleucine | 0.444 | L-Isoleucine | 0.444 | L-Isoleucine | 0.444 | L-Isoleucine | 0.444 |
| L-Valine | 0.889 | L-Valine | 0.889 | L-Valine | 0.889 | L-Valine | 0.889 |
| L-Lysine | 0.470 | L-Lysine | 0.470 | L-Lysine | 0.470 | L-Lysine HCl | 0.416 |
| Acetate | | Acetate | | Acetate | | | |
| L-Histidine | 0.333 | L-Histidine | 0.333 | L-Histidine | 0.333 | L-Histidine | 0.333 |
| L-Threonine | 0.333 | L-Threonine | 0.333 | L-Threonine | 0.333 | L-Threonine | 0.333 |
| L-Ornithine | 1.667 | L-Ornithine | 1.667 | L-Ornithine | 1.667 | L-Ornithine | 1.667 |
| LAspartate | | LAspartate | | LAspartate | | LAspartate | |
| Citric Acid | 0.600 | Citric Acid | 0.600 | Citric Acid | 0.600 | Citric Acid | 0.200 |
| Sucralose | 0.016 | Sucralose | 0.016 | Sucralose | 0.016 | Sucralose | 0.008 |
| Powder | | Powder | | Powder | | Powder | |
| Xanthan Gum | 0.080 | Xanthan Gum | 0.080 | Xanthan Gum | 0.080 | Xanthan Gum | 0.080 |
| (Tic Gums) | | (Tic Gums) | | (Tic Gums) | | (Xanatural 180) | |
| Orange flavor | 0.160 | Lecithin | 0.060 | Poloxamer 331 | 0.100 | Lecithin | 0, 0.010, |
| | | (Alcolec 40P) | | | | (Lipoid 20S) | 0.020, 0.040, |
| Custard flavor | 0.020 | Orange flavor | 0.160 | Orange flavor | 0.160 | | or 0.060 |
| FD&C Yellow | 0.0016 | Custard flavor | 0.020 | Custard flavor | 0.020 | Orange Flavor | 0.060 |
| No 6 | | FD&C Yellow | 0.0016 | FD&C Yellow | 0.0016 | FD&C Yellow | 0.0016 |
| | | No 6 | | No 6 | | No 6 | |
| TOTAL DOSE | 5.9026 | TOTAL DOSE | 5.9626 | TOTAL DOSE | 6.0026 | TOTAL DOSE | 5.3206 |
| MASS | | MASS | | MASS | | MASS | 5.3306 |
| | | | | | | | 5.3406 |
| | | | | | | | 5.3606 |
| | | | | | | | 5.3806 |

### Example 2

### Testing Wetting Agents in Aqueous Suspensions of Exemplary Dry Blended Preparation 2

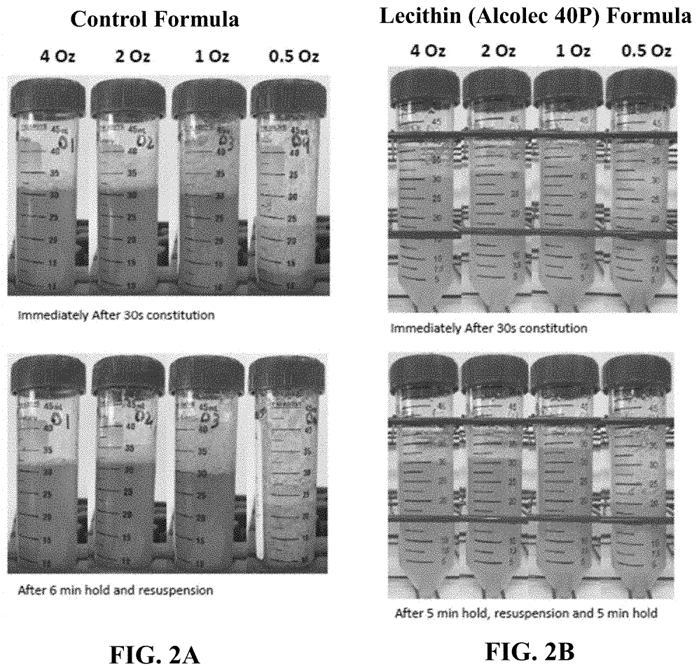

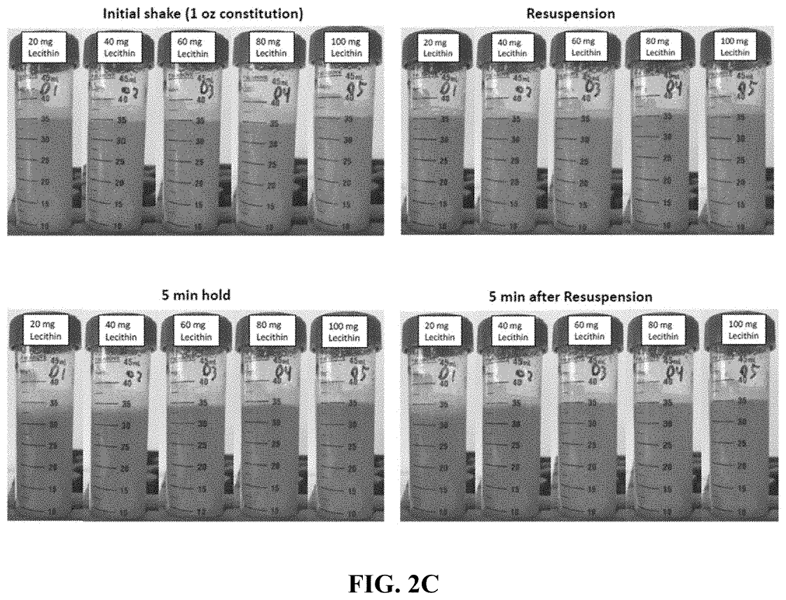

This Example describes the evaluation of the dispersion of amino acid entities in aqueous suspensions comprising exemplary dry blended preparation 2 (comprising an Active Moiety) using various wetting agents, determining whether a wetting agent improves dispersion of hydrophobic amino acids, e.g., suspension uniformity and/or foaming. First, the amino acid entities and additional components listed in Table 19 (columns 1 and 2) were mixed into a single dose of exemplary dry blended preparation 2, with or without (control) the wetting agent, lecithin (Alcolec 40P). A single dose was then added to increasing volumes of water to form aqueous suspensions. The volumes investigated were 0.5 ounces, 1 ounce, 2 ounces, and 4 ounces. The aqueous suspensions were steadily shaken for at least 30 seconds. As can be seen in FIGS. 2A and 2B, photographs of the suspensions were taken for comparison immediately following the 30 second shake and at 6 minutes of holding post-shaking (control, no wetting agent) or 5 minutes of holding post-resuspension (experimental condition with wetting agent), to visually determine by eye if the aqueous suspensions of different volumes had achieved a standard for suspension uniformity and/or the extent to which foaming (the layer of foam) had resolved. At all volumes investigated, the control aqueous suspensions as shown in FIG. 2A, that contained no additional wetting agent did not display uniform mixing and also led to the formation of foam with significant amounts of hydrophobic amino acids trapped in the foam layer for the 1 and 0.5 ounce per dose aqueous suspensions. However, the addition of 60 mg of the wetting agent lecithin (Alcolec 40P) greatly enhanced amino acid entity dispersion at all volumes tested, even at volumes as low as 0.5 ounces/dose, e.g., as seen by the smaller foam layer and more rapid decrease in the foam layer (FIG. 2B).

The ability of increasing concentrations of the wetting agent lecithin (Alcolec 40P) to increase dispersion of amino acid entities in suspensions comprising the exemplary dry blended preparation 2 was also investigated. The concentrations tested were 20 mg intervals ranging from 20 mg to 100 mg (listed in Table 19 below, third column). The aqueous suspensions were shaken for at least 30 seconds to mix. As depicted in FIG. 2C, a photograph was taken of each aqueous suspension immediately after the initial 30 second shake (top left), 5 minutes after the initial shake (bottom left), immediately after resuspension (top right), and 5 minutes of holding following resuspension (bottom right). Upon addition of greater than 20 mg of the wetting agent, lecithin (Alcolec 40P), there was noticeable improvement in amino acid entity dispersion, e.g., as seen by the smaller foam layer and more rapid decrease in the foam layer (FIG. 2C).

These data indicate that wetting agent, e.g., lecithin (Alcolec 40P), at various concentrations above 20 mg improved the dispersion of amino acid entities in aqueous suspensions of exemplary dry blended preparation 2 in volumes as low as 0.5 ounces/dose, and when included in such aqueous suspensions may allow improved dispersion of the hydrophobic amino acid entities.

TABLE 19
Components of exemplary dry blended preparation 2 with or without the wetting
agent lecithin (Alcolec 40P) investigated for improving dispersion

| | | Lecithin | | Lecithin | |
|---|---|---|---|---|---|
| Control | | (Alcolec 40P) | | (Alcolec 40P) | |
| (no wetting agent) | | at 0.06 g | | Titration | |
| Material | g/dose | Material | g/dose | Material | g/dose |
| L-Leucine | 1.000 | L-Leucine | 1.000 | L-Leucine | 1.000 |
| L-Isoleucine | 0.500 | L-Isoleucine | 0.500 | L-Isoleucine | 0.500 |
| L-Arginine HCL | 1.612 | L-Arginine HCL | 1.612 | L-Arginine HCL | 1.612 |
| L-Glutamine | 0.667 | L-Glutamine | 0.667 | L-Glutamine | 0.667 |
| L-Serine | 2.500 | L-Serine | 2.500 | L-Serine | 2.500 |
| L-Carnitine | 0.333 | L-Carnitine | 0.333 | L-Carnitine | 0.333 |
| N-Acetylcysteine | 0.433 | N-Acetylcysteine | 0.433 | N-Acetylcysteine | 0.433 |
| Citric Acid | 0.197 | Citric Acid | 0.200 | Citric Acid | 0.197 |
| Sucralose | 0.016 | Sucralose | 0.016 | Sucralose | 0.016 |
| Powder | | Powder | | Powder | |
| Xanthan Gum | 0.079 | Xanthan Gum | 0.079 | Xanthan Gum | 0.079 |
| (Tic Gums) | | (Tic Gums) | | (Tic Gums) | |
| Orange flavor | 0.079 | Lecithin | 0.060 | Lecithin | 0.020, |
| | | (Alcolec 40P) | | (Alcolec 40P) | 0.040, |
| | | | | | 0.060, |
| | | | | | 0.080, or |
| | | | | | 0.100 |
| Custard flavor | 0.012 | Orange flavor | 0.079 | Orange flavor | 0.079 |
| FD&C Yellow | 0.0079 | Custard flavor | 0.012 | Custard flavor | 0.012 |
| No 6 | | FD&C Yellow | 0.0079 | FD&C Yellow | 0.0079 |
| | | No 6 | | No 6 | |
| TOTAL DOSE | 7.436 | TOTAL DOSE | 7.495 | TOTAL DOSE | 7.456 |
| MASS | | MASS | | MASS | 7.476 |
| | | | | | 7.496 |
| | | | | | 7.516 |
| | | | | | 7.536 |

### Example 3

### Testing Wetting Agents in Aqueous Suspensions of Exemplary Dry Blended Preparation 3

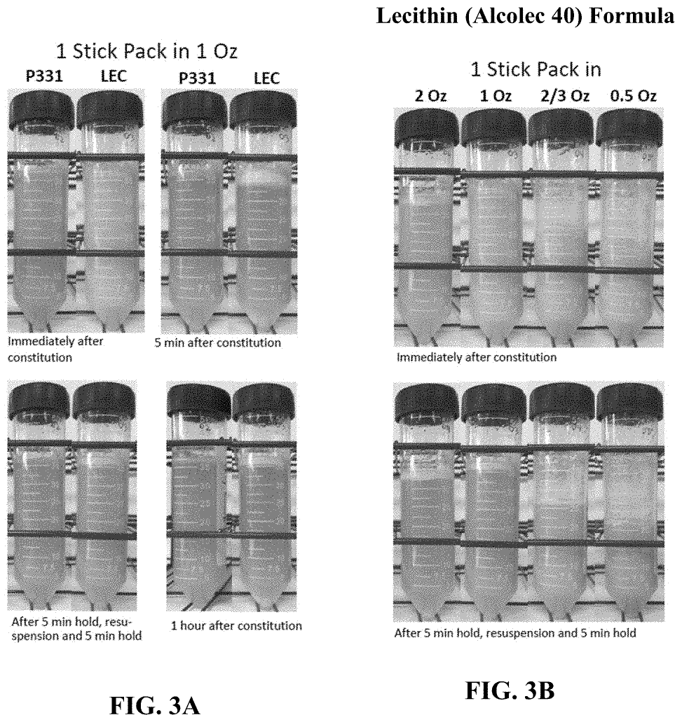

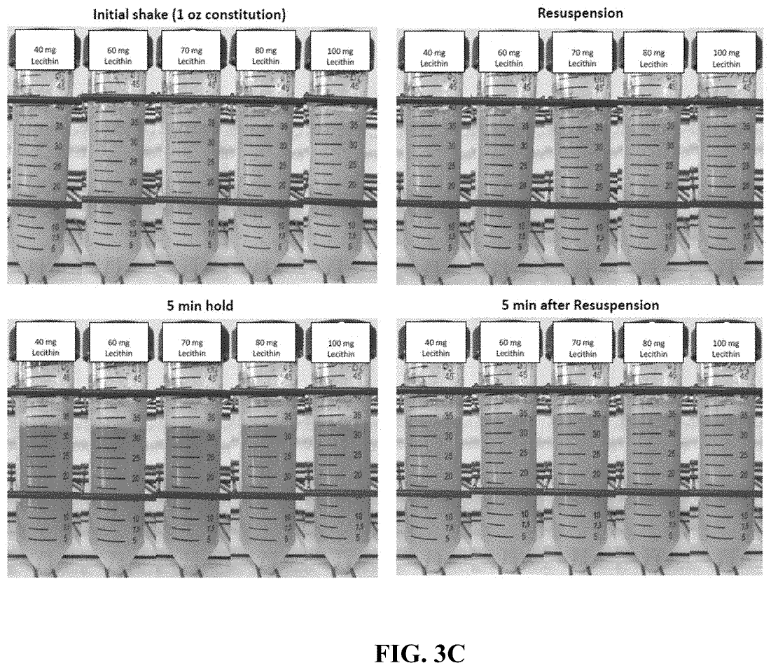

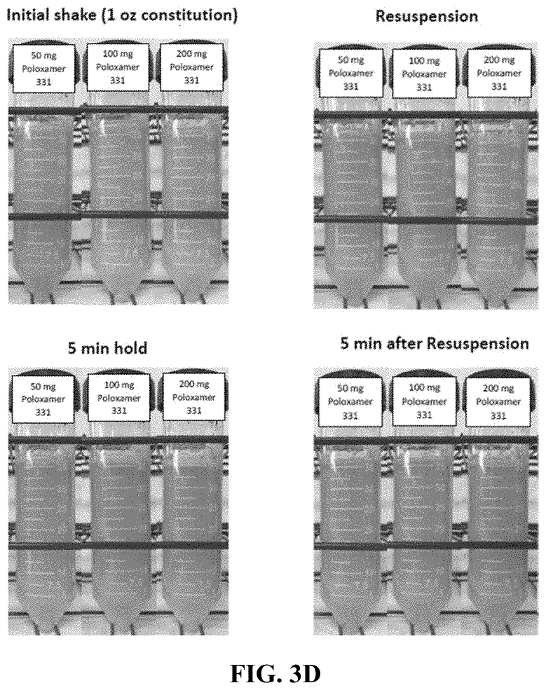

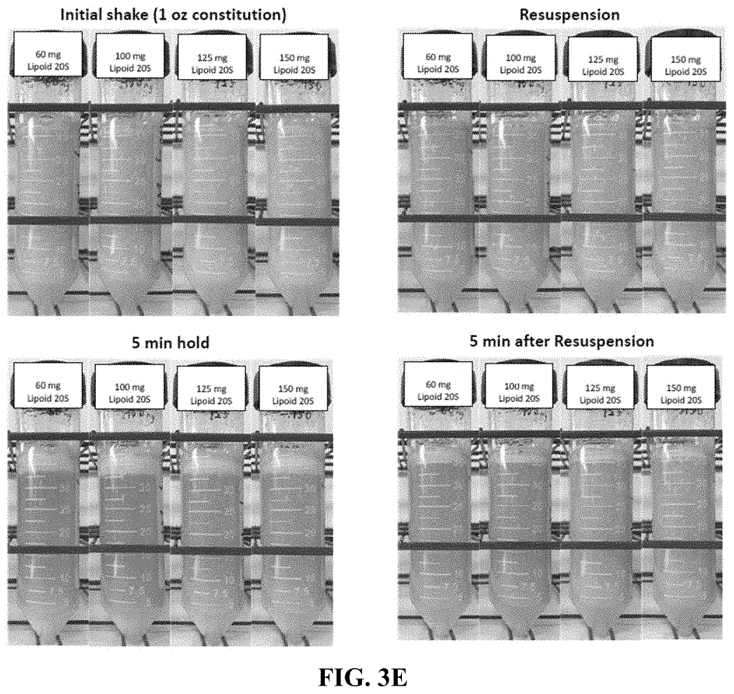

This Example describes the evaluation of the dispersion of amino acid entities in aqueous suspensions comprising exemplary dry blended preparation 3 (comprising an Active Moiety) using various wetting agents, determining whether a wetting agent improves dispersion of hydrophobic amino acids, e.g., suspension uniformity and/or foaming. First, the amino acid entities and additional components listed in Table 20 (columns 1 and 2) were mixed for a single dose of dry blended preparation 3. Control doses were constituted into water without wetting agent to create an aqueous suspension, whereas test doses of dry blended preparation had either lecithin (Alcolec 40P) included in the dry blended preparation or poloxamer 331 included in the water used to constitute the aqueous suspensions. The aqueous suspension was shaken steadily for 30 seconds. As shown in FIG. 3A, photographs of the suspension were taken immediately following constitution, 5 minutes of holding after constitution, 5 minutes of holding following resuspension, and 1 hour of holding post-constitution in order to visually determine by eye, if the aqueous suspension had achieved a standard for suspension uniformity and/or the extent to which foaming (the layer of foam) had resolved. Wetting agents poloxamer 331 or lecithin (Alcolec 40P) greatly enhanced amino acid entity dispersion, e.g., as seen by the smaller foam layer and more rapid decrease in the foam layer compared to previous experiments (FIG. 3A).

The use of 60 mg of lecithin 40 (Alcolec 40P) as a wetting agent in combination with exemplary dry blended preparation 3 was investigated in various suspension volumes ranging from 0.5, 2/3, 1, and 2 ounces of water per dose of dry blended preparation. As the volume of the suspension increased, the dispersion of the amino acid entities of the aqueous suspension comprising the wetting agent, lecithin, increased, and this was observed immediately after constitution and also after 5 minutes of holding following resuspension (FIG. 3B), as seen by the smaller foam layer and more rapid decrease in the foam layer.

As summarized in Table 20 below (columns 3 and 4), a titration experiment was also performed to determine the optimal amounts of a wetting agent, chosen from lecithin (Alcolec 40P) (LEC) or poloxamer 331 (P331), to improve amino acid dispersion in an aqueous suspension (e.g., suspension uniformity and/or foaming) comprising exemplary dry blended preparation 3. Uniformity of suspension and solubility was examined immediately after shaking for 30 seconds, after holding for 5 minutes, after resuspension and after 5 minutes of holding after resuspension. Both lecithin (Alcolec 40P) (FIG. 3C) or poloxamer 331 (P331) (FIG. 3D) had a dramatic effect on the dispersion of the amino acid entities in the aqueous suspension, as evidenced by the smaller foam layer and more rapid decrease in the foam layer, at all concentrations tested, indicating that low doses of each compound may be sufficient.

TABLE 20
Components of exemplary dry blended preparation 3 with different wetting agents, lecithin (Alcolec
40P) (LEC) or poloxamer 331 (P331) investigated at various concentrations for improving dispersion

| | | | | | | Titrating | |
|---|---|---|---|---|---|---|---|
| | | Lecithin | | Titrating | | Lecithin | |
| Poloxamer 331 | | (Alcolec 40P) | | Poloxamer 331 | | (Alcolec 40P) | |
| Material | g/dose | Material | g/dose | Material | g/dose | Material | g/dose |
| L-Arginine HCl | 2.116 | L-Arginine HCl | 2.116 | L-Arginine HCl | 2.116 | L-Arginine HCl | 2.116 |
| L-Citrulline | 1.125 | L-Citrulline | 1.125 | L-Citrulline | 1.125 | L-Citrulline | 1.125 |
| N-acetylcysteine | 0.325 | N-acetylcysteine | 0.325 | N-acetylcysteine | 0.325 | N-acetylcysteine | 0.325 |
| L-Leucine | 0.750 | L-Leucine | 0.750 | L-Leucine | 0.750 | L-Leucine | 0.750 |
| L-Histidine | 0.375 | L-Histidine | 0.375 | L-Histidine | 0.375 | L-Histidine | 0.375 |
| L-Lysine HCl | 0.467 | L-Lysine HCl | 0.467 | L-Lysine HCl | 0.467 | L-Lysine HCl | 0.467 |
| L-Valine | 0.375 | L-Valine | 0.375 | L-Valine | 0.375 | L-Valine | 0.375 |
| L-Serine | 0.937 | L-Serine | 0.937 | L-Serine | 0.937 | L-Serine | 0.937 |
| L-Glutamine | 1.875 | L-Glutamine | 1.875 | L-Glutamine | 1.875 | L-Glutamine | 1.875 |
| Levocarnitine | 0.250 | Levocarnitine | 0.250 | Levocarnitine | 0.250 | Levocarnitine | 0.250 |
| Citric Acid | 0.100 | Citric Acid | 0.100 | Citric Acid | 0.100 | Citric Acid | 0.100 |
| Xanthan Gum | 0.080 | Xanthan Gum | 0.080 | Xanthan Gum | 0.080 | Xanthan Gum | 0.080 |
| (Tic Gums) | | (Tic Gums) | | (Tic Gums) | | (Tic Gums) | |
| Sucralose | 0.015 | Sucralose | 0.016 | Sucralose | 0.015 | Sucralose | 0.015 |
| Poloxamer 331 | 0.100 | Lecithin | 0.060 | Poloxamer 331 | 0.050, | Lecithin | 0.040, |
| | | (Alcolec 40P) | | | 0.100, | (Alcolec 40P) | 0.060, |
| | | | | | 0.200 | | 0.070, |
| | | | | | | | 0.080, |
| | | | | | | | 0.100 |
| Orange flavor | 0.100 | Orange flavor | 0.075 | Orange flavor | 0.100 | Orange Flavor | 0.100 |
| Custard flavor | 0.025 | Custard flavor | 0.025 | Custard flavor | 0.025 | Custard Flavor | 0.025 |
| FD&C Yellow | 0.0015 | FD&C Yellow | 0.0015 | FD&C Yellow | 0.0015 | FD&C Yellow | 0.0015 |
| No 6 | | No 6 | | No 6 | | No 6 | |
| TOTAL DOSE | 9.017 | TOTAL DOSE | 8.953 | TOTAL DOSE | 8.967 | TOTAL DOSE | 8.957 |
| MASS | | MASS | | MASS | 9.017 | MASS | 8.977 |
| | | | | | 10.017 | | 8.987 |
| | | | | | | | 8.997 |
| | | | | | | | 9.017 |

In this example, exemplary dry blended preparation 3 was investigated with increasing concentration of the wetting agent, lecithin (Lipoid 20S), also known as Lipoid S20. The amino acid entities and the wetting agent were combined as listed in Table 21 to form a single dose of the composition, which was then added to one ounce of water to create an aqueous suspension. The aqueous suspension was steadily shaken for at least 30 seconds. In order to visually determine by eye, if the aqueous suspension had achieved a standard for suspension uniformity and/or the extent to which foaming (the layer of foam) had resolved, photographs of the aqueous suspension were taken after shaking for 30 seconds (top left), 5 minutes of holding after initial shaking (bottom left), immediately after resuspension (top right) and 5 minutes of holding after resuspension (bottom right). As depicted in FIG. 3E, 60 mg of lecithin (Lipoid 20S) was sufficient for improving dispersion of amino acids of exemplary dry blended preparation 3, e.g., as seen by the smaller foam layer and more rapid decrease in the foam layer.

These data indicate that a wetting agent, e.g., lecithin (Alcolec 40P), poloxamer 331 (P331), or lecithin (Lipoid 20S), at various concentrations improves the dispersion of amino acid entities in aqueous suspensions of exemplary dry blended preparation 3, and this when included in such aqueous suspensions may allow for improved dispersion of hydrophobic amino acid entities.

TABLE 21
Components of exemplary dry blended
preparation 3 with increasing amounts
of the wetting agent lecithin (Lipoid 20S)
investigated for improving dispersion
Titrating Lecithin (Lipoid 20S)

| Material | g/dose |
|---|---|
| L-Arginine HCl | 2.116 |
| L-Citrulline | 1.125 |
| N-acetylcysteine | 0.325 |
| L-Leucine | 0.750 |
| L-Histidine | 0.375 |
| L-Lysine HCl | 0.467 |
| L-Valine | 0.375 |
| L-Serine | 0.937 |
| L-Glutamine | 1.875 |
| Levocarnitine | 0.250 |
| Citric Acid | 0.100 |
| Xanthan Gum (Tic Gums) | 0.080 |
| Sucralose | 0.015 |
| Lecithin (Lipoid 20S) | 0.060, |
| | 0.100, |
| | 0.125, |
| | 0.150 |
| Orange Flavor | 0.100 |
| Custard Flavor | 0.025 |
| FD&C Yellow No 6 | 0.0015 |
| TOTAL DOSE MASS | 8.917 |

### Example 4

### Measuring Dispersion of Amino Acids in Aqueous Suspensions Comprising Exemplary Dry Blended Preparations Comprising an Active Moiety and a Wetting Agent

This Example describes a method for measuring dispersion of the amino acids of various exemplary dry blended preparations in aqueous suspension with or without a wetting agent in order to determine if the addition of a wetting agent leads to enhanced dispersion of hydrophobic amino acid entities compared to control suspensions that do not comprise a wetting agent. This is performed by providing dry blended preparations comprising the amino acids entities and any other additional components (no wetting agent in control doses), and either including a lecithin (e.g., Alcolec 40P or Lipoid 20S) in test dry blended preparations or including a poloxamer (e.g., poloxamer 331), e.g., in the water used to constitute the dry blended preparation, and constituting the dry blended preparations in water in order to form aqueous suspensions. Prior to mixing, a small sample is isolated from the suspension. One or more (e.g., at least 6 or more) samples (e.g., taken from one or more different areas within the suspension) are taken at a time interval following the 30 second shaking of the solution performed to mix the suspension. These time intervals may include one or more of: after initial shaking, 5 minutes of holding after shaking, 5 minutes of holding after resuspension, and one hour of holding following shaking and resuspension. The samples are tested by LCMS or HPLC with or without OPA to determine the concentrations of the amino acids in the suspension at these timepoints and/or areas of the suspension. Values are compared to one or both of i) a value from a sample taken prior to mixing of the suspension or ii) a value from a sample taken from a different area of the suspension, in order to determine if the amino acid entities of the exemplary preparations successfully and uniformly dispersed in the aqueous suspensions and if this is enhanced in suspensions comprising a wetting agent compared to control suspensions that do not comprise a wetting agent.

### Example 5

### Blending of N-Acetylcysteine (NAC) and Silicon Dioxide (SiO₂)

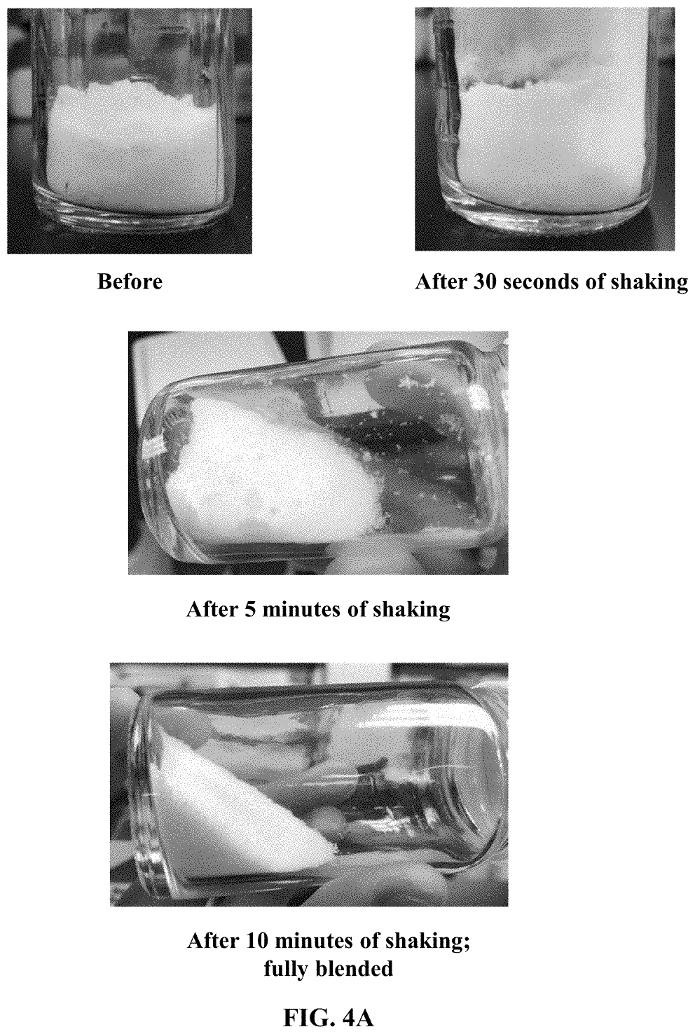

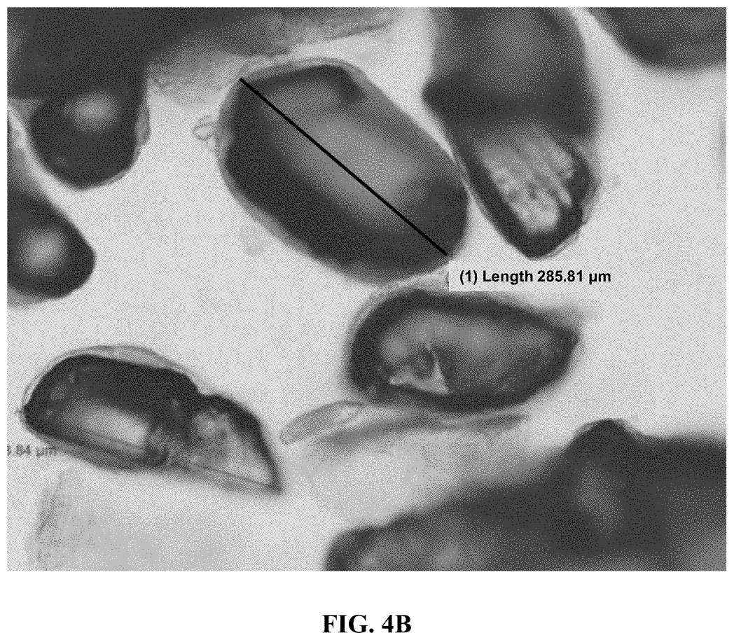

This Example describes contacting N-Acetylcysteine (NAC) particles with silicon dioxide (SiO₂) to form a mixture comprising NAC and SiO₂ (e.g., pre-treated NAC). NAC (Spectrum, AC126) at 93.5% of the total mass and SiO₂ (Aerosil 300 Pharma, Evonik) at 6.5% of the total mass were combined in a glass jar. As shown in FIG. 4A, a photograph was taken of the mixture prior to mixing. The jar was then mixed by subjecting the contents to a blending condition, e.g., shaking, with photographs taken after shaking for 30 seconds, 5 minutes, and 10 minutes (FIG. 4A). Visual microscopy of the mixture after 10 minutes of shaking confirmed that SiO₂ appeared to coat the NAC particles (FIG. 4B).

### Example 6

### Stability of an Exemplary Composition Comprising Amino Acids (e.g., an Active Moiety With or Without SiO₂

This Example describes the physical and chemical stability over time of exemplary compositions comprising amino acid entities (e.g., an Active Moiety) including NAC and an acetyl acceptor, with or without pre-treating the NAC with SiO₂ (as in Example 5). The exemplary amino acid preparation comprised: an arginine amino acid entity (L-arginine HCL (ARG-HCl)), a citrulline amino acid entity (L-Citrulline (CIT)), a glutamine amino acid entity (L-Glutamine (GLN)), a histidine amino acid entity (L-Histidine (HIS)), a leucine amino acid entity (L-Leucine (Leu)), a lysine amino acid entity (L-Lysine HCl (LYS-HCl)), a serine amino acid entity (L-Serine (SER)), a valine amino acid entity (L-Valine (Val)), an NAC amino acid entity (NAC), and and a carnitine amino acid entity (levocarnitine (CAR)). All components were blended together in a V-Blender and were then hand packaged and sealed into foil packs. If SiO₂ was included in composition, the NAC component of the powder was mixed first with SiO₂ prior to adding the additional amino acids of the exemplary composition. Packages were then stored at different temperatures and relative humidities for one month, as summarized in Table 22 and Table 23 below. Using LCMS, and HPLC with and without o-phthaladehyde (HPLC-OPA), the amino acid levels of the exemplary amino acid preparation were determined at time 0 and post-1 month of storage. As a measure of stability over time and temperature, the percent of the initial amount of each amino acid entity was calculated. A percentage of 90-110% after storage was considered to be the acceptance criteria. LCMS was also used to measure acetyl-L-carnitine (ALCAR) and cysteine production over time. Results for these assays are reported as % weight (w)/weight (w) of L-Car in the case of acetyl-L-carnitine and %w/w of NAC in the case of cysteine. These values are summarized for the exemplary amino acid preparations that include SiO₂ in Table 23 and Table 24 and those that do not contain SiO₂ in Table 23 and Table 25. The levels of NAC in the preparations comprising SiO₂ treated NAC are higher after 1 month of storage than the levels in the preparations comprising untreated NAC. The results suggested that SiO₂ stabilized NAC, decreasing degradation, e.g., by decreasing deacetylation of NAC. Additionally, no negative impact on the stability of the other amino acid components included in the preparation was detected from the addition of SiO₂.

TABLE 22
Stability of an exemplary preparation comprising
amino acids (e.g., an Active moiety) with SiO₂

| | HPLC-OPA | | | | | | | | | |
|---|---|---|---|---|---|---|---|---|---|---|
| Time Point/ | % of Initial amount (mg/dose) | | | | | | | | | |
| Content | ARG- | | | | | LYS- | | | HPLC | LCMS |
| Assay | HCL | CIT | GLN | HIS | LEU | HCL | SER | VAL | NAC | Carnitine |
| Time 0 | 108.6 | 111 | 118 | 95.7 | 106.8 | 99 | 122.6 | 97.4 | 94.1 | 103.3 |
| (initial) | | | | | | | | | | |
| 25° C./60% | 107.0 | 100.4 | 115.1 | 93.3 | 97 | 102.7 | 118.8 | 96.5 | 100.5 | 120.2 |
| RH- 1 month | | | | | | | | | | |
| 40° C./75% | 102.4 | 105.9 | 125.1 | 95.4 | 98.9 | 91.5 | 109.9 | 93.2 | 95.6 | 117.9 |
| RH- 1 month | | | | | | | | | | |
| 60° C./75% | 100.1 | 106.6 | 124.3 | 96.9 | 102.5 | 95.7 | 112.8 | 98.5 | 83.7 | 98.2 |
| RH- 1 month | | | | | | | | | | |

TABLE 23
Stability of an exemplary preparation comprising
amino acids (e.g., an Active moiety) without SiO₂

| Time Point/ | HPLC-OPA | | | | | | | | | |
|---|---|---|---|---|---|---|---|---|---|---|
| Content | ARG- | | | | | LYS- | | | HPLC | LCMS |
| Assay | HCL | CIT | GLN | HIS | LEU | HCL | SER | VAL | NAC | Carnitine |
| Time 0 | 107.2 | 97.6 | 113.3 | 94.4 | 104.5 | 93.6 | 108.1 | 107.3 | 88.8 | 111.7 |
| (initial) | | | | | | | | | | |
| 25° C./60% | 106.2 | 104.0 | 106.3 | 96.8 | 103.5 | 98.0 | 100.6 | 99.3 | 95.3 | 118.8 |
| RH- 1 month | | | | | | | | | | |
| 40° C./75% | 107.9 | 106.9 | 108.7 | 97.0 | 102.9 | 92.6 | 105.7 | 86.9 | 92.7 | 105.5 |
| RH- 1 month | | | | | | | | | | |
| 60° C./75% | 109.3 | 106.7 | 108.4 | 99.5 | 104.4 | 94.7 | 105.7 | 92.0 | 79.2 | 108.0 |
| RH- 1 month | | | | | | | | | | |

TABLE 24
Stability of an exemplary preparation comprising amino acids (e.g., an Active moiety) with SiO₂ as
measured by NAC and CAR HPLC and LCMS values at various storage conditions after 1 month of storage

| | | NAC | CAR | ALCAR | NAC | Cysteine |
|---|---|---|---|---|---|---|
| Condition | Sample | HPLC | LCMS | LCMS | LCMS | LCMS |
| 25° C./60% | exemplary preparation + SiO₂, t1 6 | 98.9% | 101.58% | 0.0015% | 95.61% | N/F |
| RH | exemplary preparation + SiO₂, t1 7 | 102% | 138.77% | 0.0014% | 98.82% | N/F |
| 40° C./75% | exemplary preparation + SiO₂, t1 30 | 95.2% | 119.37% | 0.0025% | 91.54% | N/F |
| RH | exemplary preparation + SiO₂, t1 31 | 96.0% | 116.45% | 0.0029% | 107.56% | N/F |
| 60° C./75% | exemplary preparation + SiO₂, t1 32 | 83.5% | 90.15% | 0.0648% | 88.92% | 0.4021% |
| RH | exemplary preparation + SiO₂, t1 34 | 83.9% | 106.17% | 0.0684% | 91.56% | 0.3088% |

TABLE 25
Stability of an exemplary preparation comprising amino acids (e.g.,
an Active moiety) without SiO₂ as measured by NAC and CAR HPLC and
LCMS values at various storage conditions after 1 month of storage

| | | NAC | CAR | ALCAR | NAC | Cysteine |
|---|---|---|---|---|---|---|
| Condition | Sample | HPLC | LCMS | LCMS | LCMS | LCMS |
| 25° C./60% | exemplary preparation, t1 6 | 97.56% | 116.69% | 0.0002% | 91.53% | N/F |
| RH | exemplary preparation, t1 7 | 93.14% | 120.98% | 0.0002% | 84.70% | N/F |
| 40° C./75% | exemplary preparation, t1 20 | 89.34% | 104.02% | 0.0033% | 84.15% | N/F |
| RH | exemplary preparation, t1 21 | 95.97% | 106.91% | 0.0027% | 92.28% | N/F |
| 60° C./75% | exemplary preparation, t1 32 | 76.60% | 106.80% | 0.0659% | 76.41% | N/F |
| RH | exemplary preparation, t1 34 | 78.75% | 109.21% | 0.0625% | 73.18% | N/F |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of manufacturing, making, or formulating an aqueous suspension, the method comprising:
providing a dry blended preparation comprising at least one hydrophobic amino acid entity, and a wetting agent; and
combining the dry blended preparation, the wetting agent, and water to form an aqueous suspension that achieves a standard for suspension uniformity,
wherein the aqueous suspension has a volume less than or equal to 8, 7, 6, 5, 4, 3, 2, 1, or 0.5 ounces, and one or both of:
i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce, and/or
wherein the wetting agent has a hydrophilic-lipophilic balance (HLB) value of 8-9 or 2-3,
thereby manufacturing, making, or formulating an aqueous suspension.

2. An aqueous suspension comprising:
a plurality of amino acid entities, including at least one hydrophobic amino acid entity;
a wetting agent; and
water,
wherein the aqueous suspension achieves a standard for suspension uniformity, and one or both of:
a) wherein the aqueous suspension has a volume less than or equal to 8, 7, 6, 5, 4, 3, 2, 1, or 0.5 ounces, and one or both of:
i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce, or
b) wherein the wetting agent has an HLB value of either 8-9 or 2-3.

3. A method of making a dry blended preparation with improved hydrophobic amino acid dispersion, the method comprising:
providing a dry blended preparation comprising at least one hydrophobic amino acid entity and a wetting agent,
wherein, when the dry blended preparation is combined with water to form an aqueous suspension, the aqueous suspension achieves a standard for suspension uniformity, and wherein one or both of:
a) the aqueous suspension has a volume less than or equal to 8, 7, 6, 5, 4, 3, 2, 1, or 0.5 ounces, and one or both of:
i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce, or
b) the wetting agent has an HLB value of 8-9 or 2-3, thereby making a dry blended preparation with improved hydrophobic amino acid dispersion.

4. A dry blended preparation comprising:
a plurality of amino acid entities, including at least one hydrophobic amino acid entity; and
a wetting agent with an HLB value of 8-9 or 2-3, wherein, when the dry blended preparation is combined with water to form an aqueous suspension with volume less than or equal to 8, 7, 6, 5, 4, 3, 2, 1, or 0.5 ounces, and one or both of:
i) an overall amino acid concentration of at least 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 g/ounce; or
ii) a hydrophobic amino acid concentration of at least 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.2, 2.4, 2.6, 2.8, 3, 3.2, 3.4, 3.6, 3.8, 4, 4.2, 4.4, 4.6, 4.8, or 5 g/ounce, the aqueous suspension achieves a standard for suspension uniformity.

5. The dry blended preparation of claim 4, wherein the wetting agent has an HLB value of 8, 9, 2, or 3.

6. The dry blended preparation of claim 4, wherein the wetting agent is chosen from a lecithin or a poloxamer.

7. The dry blended preparation of claim 6, wherein the wetting agent is chosen from:
i) lecithin 40P or a substantially equivalent lecithin;
ii) Lipoid 20S or a substantially equivalent lecithin;
iii) poloxamer P331 or a substantially equivalent poloxamer;
iv) a poloxamer with:
a) a polyoxypropylene core molecular mass of at least 2500, 2600, 2700, 2800, 2900, 3000, 3100, 3200, or 3300 g/mol, and/or
b) a percentage polyoxyethylene content of at least 1, 2, 3, 4, 5, 6, 7, 8, 9, or 10%; or
v) a poloxamer has a polyoxypropylene core molecular mass of about 3300 g/mol and a percentage polyoxyethylene content of about 10%.

8. The dry blended preparation of claim 4, wherein the wetting agent is a lecithin comprising one, two, or all of:
a) at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% phosphatidylcholine (PC);
b) at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% phosphatidylinositol; and
c) at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45% phosphatidylethanolamine (and optionally less than 50, 45, or 40% phosphatidylethanolamine), wherein the wetting agent is a lecithin comprising at least about 20 to 40% amphiphilic phospholipid.

9-16. (canceled)

17. The dry blended preparation of claim 4, wherein the dry blended preparation does not comprise any additional wetting agent, or wherein the aqueous suspension has a volume of about 6, 4, 2, 1, 0.5, 0.33, 0.33-0.5, 0.5-1, 1-2, 2-4, or 4-6 ounces per dose or per dosing period.

18. (canceled)

19. The dry blended preparation of claim 4, wherein the wetting agent:
a) is 0.5-4% (w/w) of the dry mixture or 0.1-1.5% (w/w) of the aqueous suspension;
b) is no more than 5, 4, 3, 2, 1.9, 1.8, 1.7, 1.6, 1.5, 1.4, 1.3, 1.2, or 1.1% (w/w) of the dry weight of the combination of the plurality of amino acid entities, wetting agent, and any other excipient;
c) is about 0.5-1.5% (w/w) of the dry weight of the combination of the plurality of amino acid entities, wetting agent, and any other excipient;
d) is at least 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, or 2.5% (w/w) of the dry weight of the combination of the plurality of amino acid entities, wetting agent, and any other excipient;
e) is no more than 5, 4, 3, 2, 1.9, 1.8, 1.7, 1.6, 1.5, 1.4, 1.3, 1.2, 1.1, 1, 0.9, 0.8, 0.7, 0.6, or 0.5% (w/w) of the dry weight of the combination of the plurality of amino acid entities, wetting agent, and any other excipient; or
f) is about 0.5-2.25% (w/w) of the dry weight of the combination of the plurality of amino acid entities, wetting agent, and any other excipient.

20-24. (canceled)

25. The method of claim 1, wherein combining comprises shaking or inverting the dry blended preparation, the wetting agent, and water, or wherein combining comprises holding the aqueous suspension, or combining produces a layer of foam at the top of the aqueous suspension.

26. The method of claim 25, wherein shaking or inverting the dry blended preparation, the wetting agent, and water has a duration of at least 10, 20, 30, 40, 50, or 60 seconds, or 1, 2, 3, 4, 5, 6, 7, 8, 9, 10, 15, 20, 25, 30, 35, 40, 45, 50, 55, or 60 minutes, or wherein shaking or inverting the dry blended preparation, the wetting agent, and water continues until the aqueous suspension appears uniform, or wherein shaking or inverting the dry blended preparation, the wetting agent, and water continues until the aqueous suspension achieves a standard for suspension uniformity.

27-30. (canceled)

31. The method of claim 25, wherein holding the aqueous suspension reduces the size of the layer of foam such that the layer of foam does not interfere with downstream processing or use by an end user of the aqueous suspension, or wherein holding the aqueous suspension has a duration of less than 20, 19, 18, 17, 16, 15, 14, 13, 12, 11, 10, 9, 8, 7, 6, 5, 4, or 3 minutes, or wherein holding continues until the aqueous suspension achieves a standard for suspension uniformity.

32-33. (canceled)

34. The dry blended preparation of claim 4, wherein the standard for suspension uniformity comprises:
a) the amount of an amino acid entity at a sampling point differing from the amount of the amino acid entity present in the aqueous suspension by less than a predetermined amount, e.g., less than 10, 9, 8, 7, 6, 5, 4, 3, 2, or 1%; or
b) the amount of an amino acid entity at a sampling point differing from the amount of the amino acid entity present at a second sampling point by less than a predetermined amount.

35. (canceled)

36. The method of claim 1, comprising acquiring a value for the amount of an amino acid entity at a sampling point and comparing the value to a reference value present in the aqueous suspension, wherein acquiring a value comprises using HPLC-UV, UPLC-UV, OPA tagged HPLC-UV, Accqtag HPLC-UV, and/or LC/MS.

37-38. (canceled)

39. The dry blended preparation of claim 4, wherein a solubilization rate of an amino acid of the aqueous suspension is increased relative to a similar suspension not comprising the wetting agent.

40. The dry blended preparation of claim 4, wherein:
a) the hydrophobic amino acid entity is a branched chain amino acid (BCAA);
b) the hydrophobic amino acid entity is chosen from a leucine amino acid entity, a valine amino acid entity, an isoleucine amino acid entity, or a tryptophan amino acid entity;
c) the dry blended preparation comprises one, two, three, or all of a leucine amino acid entity, a valine amino acid entity, an isoleucine amino acid entity, or a tryptophan amino acid entity;
d) the dry blended preparation comprises a leucine amino acid entity, an arginine amino acid entity, a glutamine amino acid entity, a citrulline amino acid entity, a serine amino acid entity, a valine amino acid entity, a histidine amino acid entity, a lysine amino acid entity, a N-acetylcysteine (NAC) amino acid entity, and a carnitine amino acid entity;
e) the dry blended preparation comprises a leucine amino acid entity, an isoleucine amino acid entity, a valine amino acid entity, an arginine amino acid entity, a glutamine amino acid entity, and a N-acetylcysteine (NAC) entity; or
f) the dry blended preparation comprises a leucine amino acid entity, an isoleucine amino acid entity, a valine amino acid entity, a histidine amino acid entity, a lysine amino acid entity, a threonine amino acid entity, an ornithine amino acid entity, and an aspartate amino acid entity.

41-45. (canceled)

46. The dry blended preparation of claim 40, wherein the dry blended composition further comprises one or more pharmecutically acceptable excipients.

47. (canceled)

48. A method of stabilizing N-acetylcysteine (NAC), e.g., in the presence of carnitine (CAR), the method comprising:
a) forming a dry blended preparation comprising NAC and CAR, under conditions such that deacetylation of NAC in the dry blended preparation is decreased compared to the deacetylation in a reference mixture,
wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water,
thereby stabilizing NAC; or
b) forming a dry blended preparation comprising NAC and CAR, under conditions such that deacetylation of NAC in the dry blended preparation is decreased compared to the deacetylation in a reference mixture,
wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water,
thereby making an dry blended preparation comprising NAC and an CAR.

49. (canceled)

50. The method of claim 48, wherein the conditions comprise one or more:
i) the dry blended preparation comprises an adsorbent and the deacetylation of NAC in the dry blended preparation is decreased compared to the deacetylation in a reference mixture comprising NAC and CAR in the absence of the adsorbent;
ii) maintaining a concentration in the dry blended preparation of less than 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w);
iii) CAR is provided in a form with reduced hygroscopicity relative to the free-base form of CAR; or
iv) the dry blended preparation comprises one or more other components in a form with reduced hygroscopicity.

51. (canceled)

52. A dry blended preparation comprising:
a) N-acetylcysteine (NAC), carnitine (CAR) and an adsorbent,
wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, and
wherein the adsorbent (e.g., SiO₂) is present at a weight percentage (w/w) of at least 0.05, 0.075, 0.1, 0.125, 0.15, 0.175, 0.2, 0.225, 0.25, 0.275, 0.3, 0.325, 0.35, 0.4, 0.425, 0.45, 0.475, 0.5, 0.6, 0.7, 0.8, 0.9, 1, 1.5, 2, 2.5, 3, 3.5, 4, 4.5, or 5%, e.g., at least 0.3%; or
b) N-acetylcysteine (NAC) and an adsorbent,
wherein the dry blended preparation comprises less than 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% (w/w) water, and
wherein the adsorbent is present at a weight percentage (w/w) of at least 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, 10, 12, 14, 16, 18, 20, 22, 24, 26, 28, 30, 32, 34, 36, 38, or 40%.

53. (canceled)

54. The dry blended preparation of claim 52, wherein the adsorbent:
is chosen from: SiO₂, magnesium silicate, calcium silicate, Talc, calcium carbonate, magnesium carbonate, MgO, calcium sulfate, CaCl₂, aluminum metal silicate, anhydrous Si acid, magnesium aluminum silicate, microcrystalline cellulose, sodium carboxymethylcellulose, calcium carboxymethylcellulose or any other appropriate adsorbent;
b) is present in the mixture of NAC and adsorbent at a weight percentage (w/w) of at least 1, 1.5, 2, 2.5, 3, 3.5, 4, 4.5, 5, 5.5, 6, 6.1, 6.2, 6.3, 6.4, 6.5, 6.6, 6.7, 6.8, 6.9, 7, 7.5, 8, 8.5, 9, 9.5, or 10, 12, 14, 16, 18, 20, 22, 24, 26, 28, 30, 32, 34, 36, 38, or 40%;
c) is present in the dry blended preparation at a weight percentage (w/w) of less than or equal to 10, 9, 8, 7, 6, 5, 4, 3, 2, 1.5, 1, 0.9, 0.8, 0.7, 0.6, 0.575, 0.55, 0.525, 0.5, 0.475, 0.45, 0.425, 0.4, 0.375, 0.35, or 0.325%.

55-56. (canceled)

57. The dry blended preparation of claim 52,
wherein the dry blended preparation further comprises:
a) a leucine (L)-amino acid entity, an arginine (R)-amino acid entity, and/or a glutamine (Q)-amino acid entity;
b) a leucine (L)-amino acid entity, an arginine (R)-amino acid entity; and a glutamine (Q)-amino acid entity;
c) a leucine (L)-amino acid entity, an arginine (R)-amino acid entity; a glutamine (Q)-amino acid entity, a citrulline amino acid entity, a serine (S)-amino acid entity, a valine (V)-amino acid entity, a histidine (H)-amino acid entity and a lysine (K)-amino acid entity; or
d) a leucine (L)-amino acid entity, an arginine (R)-amino acid entity, a glutamine (Q)-amino acid entity, an isoleucine (I)-amino acid entity, and a serine (S)-amino acid entity.

58-60. (canceled)
